SEQUENCE LISTING (256 sequences)

SEQ ID NO: 1 - protein, 22 aa, Homo sapiens
FNNKRRLRRPYYPRKALLCYQL

SEQ ID NO: 2 - protein, 16 aa, Homo sapiens
FNNGIGRHKTYLCYEV

SEQ ID NO: 3 - protein, 19 aa, Homo sapiens
FNFNNDPLVLRRRQTYLCY

SEQ ID NO: 4 - protein, 10 aa, Homo sapiens
FENKKKCHAE

SEQ ID NO: 5 - protein, 18 aa, Homo sapiens
LHNQAKNLLCGFYGRHAE

SEQ ID NO: 6 - protein, 18 aa, Homo sapeins
LCNEAKNLLCGFYGRHAE

SEQ ID NO: 7 - protein, 20 aa, Homo sapiens
LRIFASRLYYHWCKPQQDGL

SEQ ID NO: 8 - protein, 19 aa, Homo sapiens
LRIFAARIYDYDPLYKEAL

SEQ ID NO: 9 - protein, 27 aa, Homo sapiens
FNPYKMLEELDKNSRAIKRRLDRIKQS

SEQ ID NO: 10 - protein, 27 aa, Homo sapiens
FQPWDGLDEHSQALSGRLRAILQNQGN

SEQ ID NO: 11 - protein, 27 aa, Homo sapiens
FQPWDGLEEHSQALSGRLRAILQNQGN

SEQ ID NO: 12 - protein, 19 aa, Homo sapiens
FNNKRRLRRPYYPRKALLC

SEQ ID NO: 13 - protein, 19 aa, Pan troglodytes
FNNRRRLRRPYYPRKALLC

SEQ ID NO: 14 - protein, 19 aa, Gorilla gorilla
FNNKLRLRRPYYRRKTLLC

SEQ ID NO: 15 - protein, 19 aa, Pongo pygmaeus
FNNKRRIKRPYYRPKALLC

SEQ ID NO: 16 - protein, 19 aa, Symphalangus syndactylus
FNNKRRVTKPYYPRKALLC

SEQ ID NO: 17 - protein, 19 aa, Chlorocebus sabaeus
FNNKRHVSKPYYPRKALLC

SEQ ID NO: 18 - protein, 19 aa, Papio anubis
FNNKRRVNKPYYPRKALLC

SEQ ID NO: 19 - protein, 8 aa, Homo sapiens
NKKKCHAE

SEQ ID NO: 20 - protein, 8 aa, Symphalangus syndactylus
NKKKRHAE

SEQ ID NO: 21 - protein, 8 aa, Chlorocebus sabaeus
NKKKHHAE

SEQ ID NO: 22 - protein, 8 aa, Papio anubis
NKKKNHAE

SEQ ID NO: 23 - protein, 8 aa, Macaca mulatta
NKKKDHAE

SEQ ID NO: 24 - protein, 8 aa, Macaca nemestrina
NKKEDHAE

SEQ ID NO: 25 - protein, 18 aa, Homo sapiens
IFASRLYYHWCKPQQKGL

SEQ ID NO: 26 - protein, 18 aa, Pan troglogytes
IFASRLYYHWCKPQQEGL

SEQ ID NO: 27 - protein, 18 aa, Gorilla gorilla
IFASRLYYHWCKRQQEGL

SEQ ID NO: 28 - protein, 18 aa, Pongo pygmaeus
IFASRLYCHWCRRQQEGL

SEQ ID NO: 29 - protein, 18 aa, Chlorocebus sabaeus
IFASRLYYHWRPHYQEGL

SEQ ID NO: 30 - protein, 18 aa, Papio anubis
IFASRLYYHWRPNYQEGL

SEQ ID NO: 31 - protein, 18 aa, Macaca mulatta
IFASRLYYHWRPNYQEGL

SEQ ID NO: 32 - protein, 22 aa, Homo sapiens
MLEELDKNSRAIKRRLDRIKQS

SEQ ID NO: 33 - protein, 22 aa, Pan troglodytes
MLEELDKNSRAIKRRLERIKQS

SEQ ID NO: 34 - protein, 21 aa, Gorilla gorilla
MLEELDKNSQAIKRRLERIKS

SEQ ID NO: 35 - protein, 22 aa, Pongo pygmaeus
MLEELDKNSRAIKRRLERIKQS

SEQ ID NO: 36 - protein, 21 aa, Symphalangus syndactylus
MLEELDKNSRAIKRRLEKIKS

SEQ ID NO: 37 - protein, 22 aa, Chlorocebus sabaeus
KLEELDKNSRAIKRRLERIEQS

SEQ ID NO: 38 - protein, 21 aa, Papio anubis
KLEELDKNSRAIKRRLERIKS

SEQ ID NO: 39 - protein, 21 aa, Macaca mulatta
KLKELDKNSQAIKRRLERIKS

SEQ ID NO: 40 - protein, 22 aa, Macaca nemestrina
KLEELDKNSQAIKRRLERIKQS

SEQ ID NO: 41 - protein, 19 aa, Sus scrofa
FGNQPRVLAPYYLRKTYLC

SEQ ID NO: 42 - protein, 19 aa, Bos taurus
FGNQPRVPAPYYRRKTYLC

SEQ ID NO: 43 - protein, 19 aa, Ovis aries
FGNQPRVPPPYYRRKTYLC

SEQ ID NO: 44 - protein, 19 aa, Equus caballus
FGNQRRVTKPYYRRKTYVC

SEQ ID NO: 45 - protein, 18 aa, Canis familiaris
FSNQRVPKPTYQRRTYLC

SEQ ID NO: 46 - protein, 19 aa, Felis catus
FGNQHRVPKPYYRRKTYLC

SEQ ID NO: 47 - protein, 8 aa, Bos Taurus
NKKQRHAE

SEQ ID NO: 48 - protein, 8 aa, Canis familiaris
NQEKRHAE

SEQ ID NO: 49 - protein, 18 aa, Sus scrofa
LFASRLYFHWVRCYQRGL

SEQ ID NO: 50 - protein, 18 aa, Bos taurus
IFASRLYFHWIKSFKMGL

SEQ ID NO: 51 - protein, 18 aa, Ovis aries
IFASRLYFHWIKPFCRGL

SEQ ID NO: 52 - protein, 18 aa, Equus caballus
IFVSRLYYHWFRQNQQGL

SEQ ID NO: 53 - protein, 18 aa, Canis familiaris
LFASRLYFHWRQKYKREL

SEQ ID NO: 54 - protein, 18 aa, Felis catus
IFASRLYVHWRWKYQQGL

SEQ ID NO: 55 - protein, 22 aa, Sus scrofa
SWEKLEQYSESISRRLSRILRF

SEQ ID NO: 56 - protein, 22 aa, Bos taurus
PWDKLEQYSASIRRRLQRILTA

SEQ ID NO: 57 - protein, 22 aa, Equus caballus
SWEKLTEYSKGIKRRLQKILEQ

SEQ ID NO: 58 - protein, 22 aa, Canis familiaris
PWNKLKQYSESIGRRLQRILQP

SEQ ID NO: 59 - protein, 22 aa, Felis catus
PWPNLDQYSKSIKRRLGKILTP

SEQ ID NO: 60 - protein, 16 aa, Homo sapiens
FKNLWEANDRNETWLC

SEQ ID NO: 61 - protein, 8 aa, Homo sapiens
SETHCHAE

SEQ ID NO: 62 - protein, 18 aa, Homo sapiens
IFTARLYYFQYPCYQEGL

SEQ ID NO: 63 - protein, 21 aa, Homo sapiens
PWKGLKTNFRLLKRRLRESLQ

SEQ ID NO: 64 - protein, 16 aa, Homo sapiens
FKNVRWAKGRRETYLC

SEQ ID NO: 65 - protein, 18 aa, Homo sapiens
IFASRLYYHWCKPQQKGL

SEQ ID NO: 66 - protein, 18 aa, Homo sapiens
IFASRLYYHWCKPQQKGL

SEQ ID NO: 67 - protein, 19 aa, Homo sapiens
IFTARLYFCEDRKAEPEGL

SEQ ID NO: 68 - protein, 22 aa, Homo sapiens
AWEGLHENSVRLSRQLRRILLP

SEQ ID NO: 69 - DNA, 103 bp, Artificial Sequence
  synthetic oligonucleotide
acacccccatcggcgacggccccgtgctgctgcccgacaaccactattcaagtacccagtcggccctgagcaaagaccccaacgagaagcgcgatcacatggt

SEQ ID NO: 70 - DNA, 103 bp, Artificial Sequence
  synthetic oligonucleotide
acacccccatcggcgacggccccgtgctgctgcccgacaaccactatttaagtacccagtcggccctgagcaaagaccccaacgagaagcgcgatcacatggt

SEQ ID NO: 71 - DNA, 103 bp, Artificial Sequence
  synthetic oligonucleotide
acacccccatcggcgacggccccgtgctgctgcccgacaaccattatttaagtacccagtcggccctgagcaaagaccccaacgagaagcgcgatcacatggt

SEQ ID NO: 72 - DNA, 103 bp, Artificial Sequence
  synthetic oligonucleotide
acacccccatcggcgacggccccgtgctgctgcccgacaaccattattcaagtacccagtcggccctgagcaaagaccccaacgagaagcgcgatcacatggt

SEQ ID NO: 73 - DNA, 103 bp, Artificial Sequence
  synthetic oligonucleotide
acacccccatcggcgacggccccgtgctgctgcccgacaactattatttaagtacccagtcggccctgagcaaagaccccaacgagaagcgcgatcacatggt

SEQ ID NO: 74 - DNA, 90 bp, Artificial Sequence
  synthetic oligonucleotide
acacccccatcggcgacggccccgtgctgctgcccgacaaccacagtcggccctgagcaaagaccccaacgagaagcgcgatcacatggt

SEQ ID NO: 75 - DNA, 103 bp, Artificial Sequence
  synthetic oligonucleotide
accgcatcgagctgaagggcatcgacttcaaggaggacggcaacatttcagggcacaagctggagtacaactacaacagccacaacgtctatatcatggccga

SEQ ID NO: 76 - DNA, 103 bp, Artificial Sequence
  synthetic oligonucleotide
accgcatcgagctgaagggcatcgacttcaaggaggacggcaacattttagggcacaagctggagtacaactacaacagccacaacgtctatatcatggccga

SEQ ID NO: 77 - DNA, 103 bp, Artificial Sequence
  synthetic oligonucleotide
accgcatcgagctgaagggcattgactttaaaaaaaacggcaacattttagggcacaagctggagtacaactacaacagccacaacgtctatatcatggccga

SEQ ID NO: 78 - DNA, 103 bp, Artificial Sequence
  synthetic oligonucleotide
accgcatcgagctgaagggcatcgacttcaaggaggacggcaatattttagggcacaagctggagtacaactacaacagccacaacgtctatatcatggccga

SEQ ID NO: 79 - DNA, 95 bp, Artificial Sequence
  synthetic oligonucleotide
accgcatcgagctgaagggcatcgacttcaaggaggacggcaacatttcagctggagtacaactacaacagccacaacgtctatatcatggccga

SEQ ID NO: 80 - DNA, 103 bp, Artificial Sequence
  synthetic oligonucleotide
accgcatcgagctgaagggcatcgacttcaaggaggacggcaatatttcagggcacaagctggagtacaactacaacagccacaacgtctatatcatggccga

SEQ ID NO: 81 - protein, 183 aa, Homo sapiens [sequence omitted]

SEQ ID NO: 82 - DNA, 552 bp, Homo sapiens
atggctctgttaacagccgaaacattccgcttacagtttaacaacaagcgccgcctcagaaggccttactacccgaggaaggccctcttgtgttaccagctgacgccgcagaatggctccacgcccaccagaggctactttgaaaacaagaaaaagtgccatgcagaaatttgctttattaacgagatcaagtccatgggactggacgaaacgcagtgctaccaagtcacctgttacctcacgtggagcccctgctcctcctgtgcctgggagctggttgacttcatcaaggctcacgaccatctgaacctgcgcatcttcgcctcccgcctgtactaccactggtgcaagccccagcaggacgggctgcggcttctgtgtggatcccaggtcccggtggaggtcatgggcttcccagagtttgctgactgctgggaaaactttgtggaccacgagaaaccgctttccttcaacccctataagatgttagaggagctagataaaaacagtcgagccataaagcgacggcttgacaggataaagcagtcctga

SEQ ID NO: 83 - protein, 183 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKQS

SEQ ID NO: 84 - DNA, 1070 bp, Homo sapiens
tgacttttgggagagctgaccttttgtgacttttgggagagctgccaaaagtgaaacttagtgcctcagacaagcaggggcaagtctgctaaggaagctgtggccagaagcacagatcagaaacacgatggctctgttaacagccgaaacattccgcttacagtttaacaacaagcgccgcctcagaaggccttactacccgaggaaggccctcttgtgttaccagctgacgccgcagaatggctccacgcccacgagaggctactttgaaaacaagaaaaagtgccatgcagaaatttgctttattaacgagatcaagtccatgggactggacgaaacgcagtgctaccaagtcacctgttacctcacgtggagcccctgctcctcctgtgcctgggagctggttgacttcatcaaggctcacgaccatctgaacctgggcatcttcgcctcccgcctgtactaccactggtgcaagccccagcagaaggggctgcggcttctgtgtggatcccaggtcccggtggaggtcatgggcttcccagagtttgctgactgctgggaaaactttgtggaccacgagaaaccgctttccttcaacccctataagatgttagaggagctagataaaaacagtcgagccataaagcgacggcttgagaggataaagcagtcctgaagtgtggatgttttagagaatgacttaagaagtttgcagcttggacccgtatcccactcattatcaagaagcaactcaagatgactttccctggggcatgtcagttgcctcatagcctgctggtcctgtaagcaagcactaagctccacagtgccagttccttgccccaacctggccccatccaagtacagaagaccttcctttcctcctttttccatattgctttctgttctaagtgggtgaataattttataattgaaaaaataaagataaagtctgtaaatccagccgggtacggtggctcacgcctgtaatcctagcactttgggaggctgagatgctcggccaataaatttctattgtttatgaaaaaaaaaaaaaaaaaaaaaaa

SEQ ID NO: 85 - DNA, 4104 bp, Streptococcus pyogenes
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttggcagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaatctacaatcaattatttgaagaaaaccctattaacgcaagtagagtagatgctaaagcgattctttctgcacgattgagtaaatcaagacgattagaaaatctcattgctcagctccccggtgagaagagaaatggcttgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcgcctaccatgatttgctaaaaattattaaagataaagattttttggataatgaagaaaatgaagatatcttagaggatattgttttaacattgaccttatttgaagatagggggatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaggtgtctggacaaggccatagtttacatgaacagattgctaacttagctggcagtcctgctattaaaaaaggtattttacagactgtaaaaattgttgatgaactggtcaaagtaatggggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctacaaaatggaagagacatgtatgtggaccaagaattagatattaatcgtttaagtgattatgatgtcgatcacattgttccacaaagtttcattaaagacgattcaatagacaataaggtactaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 86 - protein, 1367 aa, Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 87 - protein, 1368 aa, Artificial Sequence
  synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 88 - protein, 1368 aa, Artificial Sequence
  synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 89 - protein, 1368 aa, Artificial Sequence
  synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 90 - protein, 5 aa, Artificial Sequence
  synthetic polypeptide
GGGGS

SEQ ID NO: 91 - protein, 5 aa, Artificial Sequence
  synthetic polypeptide
EAAAK

SEQ ID NO: 92 - protein, 16 aa, Artificial Sequence
  synthetic polypeptide
SGSETPGTSESATPES

SEQ ID NO: 93 - protein, 9 aa, Artificial Sequence
  synthetic polypeptide
LAGLIDADG

SEQ ID NO: 94 - DNA, 43 bp, Artificial Sequence
  synthetic oligonucleotide
gcgcggtctctaggtggcggcggcatggctctgttaacagccg

SEQ ID NO: 95 - DNA, 29 bp, Artificial Sequence
  synthetic oligonucleotide
gcgctctagattaggactgctttatcctc

SEQ ID NO: 96 - DNA, 56 bp, Artificial Sequence
  synthetic oligonucleotide
ggatccgaattcgctggaagttctgttccaggggatggtgagcaagggcgaggagg

SEQ ID NO: 97 - DNA, 56 bp, Artificial Sequence
  synthetic oligonucleotide
ctccaccggcggcatggacgccgctgccatggctctgttaacagccgaaacattcc

SEQ ID NO: 98 - DNA, 56 bp, Artificial Sequence
  synthetic oligonucleotide
ggaatgtttcggctgttaacagagccatggcagcggcgtccatgccgccggtggag

SEQ ID NO: 99 - DNA, 29 bp, Artificial Sequence
  synthetic oligonucleotide
gcgcgcggccgctcaggactgctttatcc

SEQ ID NO: 100 - DNA, 29 bp, Artificial Sequence
  synthetic oligonucleotide
  misc_feature (1)..(4): n is a, c, g, or t
nnnnggtaccaccatggctctgttaacag

SEQ ID NO: 101 - DNA, 39 bp, Artificial Sequence
  synthetic oligonucleotide
aaacatctcctggactcaccttgttttcaaagtagcctc

SEQ ID NO: 102 - DNA, 40 bp, Artificial Sequence
  synthetic oligonucleotide
gtctcctttcatctcaacagaaaaagtgccatgcggaaat

SEQ ID NO: 103 - DNA, 30 bp, Artificial Sequence
  synthetic oligonucleotide
  misc_feature (1)..(4): n is a, c, g, or t
nnnngcggccgctcaggactgctttatcct

SEQ ID NO: 104 - DNA, 39 bp, Artificial Sequence
  synthetic oligonucleotide
gaggctactttgaaaacaaggtgagtccaggagatgttt

SEQ ID NO: 105 - DNA, 40 bp, Artificial Sequence
  synthetic oligonucleotide
atttccgcatggcactttttctgttgagatgaaaggagac

SEQ ID NO: 106 - DNA, 40 bp, Artificial Sequence
  synthetic oligonucleotide
tgatccgcggccgcaccaccatggtgagcaagggcgagga

SEQ ID NO: 107 - DNA, 49 bp, Artificial Sequence
  synthetic oligonucleotide
cggctgttaacagagccatggcagcggcgtccatgccaccggtggagtg

SEQ ID NO: 108 - DNA, 49 bp, Artificial Sequence
  synthetic oligonucleotide
cactccaccggtggcatggacgccgctgccatggctctgttaacagccg

SEQ ID NO: 109 - DNA, 36 bp, Artificial Sequence
  synthetic oligonucleotide
attaagcgtacgtcaggactgctttatcctgtcaag

SEQ ID NO: 110 - DNA, 43 bp, Artificial Sequence
  synthetic oligonucleotide
attattattattctaatggatttatttatttatttatttattt

SEQ ID NO: 111 - protein, 154 aa, Homo sapiens [sequence omitted]

SEQ ID NO: 112 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLRIFASRLYYHWCKPQQDGLRLLCGSQVPVEVMGFPEFADCWENFVDHEKPLSFNPYKMLEELDKNSRAIKRRLDRIKIPGVRAQGRYMDILCDAEV

SEQ ID NO: 113 - protein, 182 aa, Homo sapiens [sequence omitted]

SEQ ID NO: 114 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKIPGVRAQGRYMDILCDAEV

SEQ ID NO: 115 - protein, 182 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKS

SEQ ID NO: 116 - protein, 183 aa, Homo sapiens; identical to SEQ ID NO: 83

SEQ ID NO: 117 - protein, 200 aa, Homo sapiens; identical to SEQ ID NO: 114

SEQ ID NO: 118 - protein, 154 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPDSRGTCAGSLHGYIV

SEQ ID NO: 119 - protein, 182 aa, Homo sapiens; identical to SEQ ID NO: 115

SEQ ID NO: 120 - protein, 183 aa, Homo sapiens; identical to SEQ ID NO: 83

SEQ ID NO: 121 - protein, 200 aa, Homo sapiens; identical to SEQ ID NO: 114

SEQ ID NO: 122 - protein, 182 aa, Homo sapiens [sequence omitted]

SEQ ID NO: 123 - protein, 200 aa, Homo sapiens; identical to SEQ ID NO: 114

SEQ ID NO: 124 - protein, 203 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKGLLLVLLDSRGTCAGSLHGYIV

SEQ ID NO: 125 - protein, 203 aa, Homo sapiens; identical to SEQ ID NO: 124

SEQ ID NO: 126 - protein, 200 aa, Homo sapiens; identical to SEQ ID NO: 114

SEQ ID NO: 127 - protein, 182 aa, Homo sapiens; identical to SEQ ID NO: 115

SEQ ID NO: 128 - DNA, 1444 bp, Homo sapiens
ggagaaggggtggggcagggtatcgctgactcagcagcttccaggttgctctgatgatatattaaggctcctgaatcctaagagaatgttggtgaagatcttaacaccacgccttgagcaagtcgcaagagcgggaggacacagaccaggaaccgagaagggacaagcacatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacgccagcaaagcagtatgctcccgatcaagtagatttttaaaaaatcagagtgggccgggcgcggtggctcacgcctgtaatcccagcactttggaggccaaggcgggtggatcacgaggtcaggagatcgagaccatcctggctaacacggtgaaaccctgtctctactaaaaatacaaaaaattagccaggcgtggtggcgggcgcctgtagtcccagctactctggaggctgaggcaggagagtagcgtgaacccgggaggcagagcttgcggtgagccgagattgcgctactgcactccagcctgggcgacagtaccagactccatctcaaaaaaaaaaaaaccagactgaattaattttaactgaaaatttctcttatgttccaagtacacaatagtaagattatgctcaatattctcagaataattttcaatgtattaatgaaatgaaatgataatttggcttcatatctagactaacacaaaattaagaatcttccataattgcttttgctcagtaactgtgtcatgaattgcaagagtttccacaaacact

SEQ ID NO: 129 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 130 - DNA, 1560 bp, Homo sapiens
cacagagcttcaaaaaaagagcgggacagggacaagcgtatctaagaggctgaacatgaatccacagatcagaaatccgatggagcggatgtatcgagacacattctacgacaactttgaaaacgaacccatcctctatggtcggagctacacttggctgtgctatgaagtgaaaataaagaggggccgctcaaatctcctttgggacacaggggtctttcgaggccaggtgtatttcaagcctcagtaccacgcagaaatgtgcttcctctcttggttctgtggcaaccagctgcctgcttacaagtgtttccagatcacctggtttgtatcctggaccccctgcccggactgtgtggcgaagctggccgaattcctgtctgagcaccccaatgtcaccctgaccatctctgccgcccgcctctactactactgggaaagagattaccgaagggcgctctgcaggctgagtcaggcaggagcccgcgtgaagatcatggactatgaagaatttgcatactgctgggaaaactttgtgtacaatgaaggtcagcaattcatgccttggtacaaattcgatgaaaattatgcattcctgcaccgcacgctaaaggagattctcagatacctgatggatccagacacattcactttcaactttaataatgaccctttggtccttcgacggcgccagacctacttgtgctatgaggtggagcgcctggacaatggcacctgggtcctgatggaccagcacatgggctttctatgcaacgaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgcatcttcgctgcccgcatctatgattacgaccccctatataaggaggcgctgcaaatgctgcgggatgctggggcccaagtctccatcatgacctacgatgagtttgagtactgctgggacacctttgtgtaccgccagggatgtcccttccagccctgggatggactagaggagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacaccagcaaagcaatgtgctcctgatcaagtagattttttaaaaatcagagtcaattaattttaattgaaaatttctcttatgttccaagtgtacaagagtaagattatgctcaatattcccagaatagttttcaatgtattaatgaagtgattaattggctccatatttagactaataaaacattaagaatcttccataattgtttccacaaacactaaaaaaaaaaaaaaaaaaaaaaa

SEQ ID NO: 131 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 132 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 133 - protein, 187 aa, Homo sapiens
MDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 134 - protein, 190 aa, Homo sapiens
MDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 135 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 136 - protein, 198 aa, Homo sapiens [sequence omitted]

SEQ ID NO: 137 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 138 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 139 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 140 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 141 - DNA, 44 bp, Artificial Sequence
  synthetic oligonucleotides
ctgttaacagccgaaacattcgagttacagtttaacaacaagcg

SEQ ID NO: 142 - DNA, 26 bp, Artificial Sequence
  synthetic oligonucleotides
cagtttaacaacgagcgccgcctcag

SEQ ID NO: 143 - DNA, 40 bp, Artificial Sequence
  synthetic oligonucleotides
cttacagtttaacaacaaggaacgcctcagaaggccttac

SEQ ID NO: 144 - DNA, 39 bp, Artificial Sequence
  synthetic oligonucleotides
cagtttaacaacaagcgcgaactcagaaggccttactac

SEQ ID NO: 145 - DNA, 28 bp, Artificial Sequence
  synthetic oligonucleotides
caagcgccgcctcgaaaggccttactac

SEQ ID NO: 146 - DNA, 40 bp, Artificial Sequence
  synthetic oligonucleotides
caacaagcgccgcctcagagaaccttactacccgaggaag

SEQ ID NO: 147 - DNA, 26 bp, Artificial Sequence
  synthetic oligonucleotides
cgcctcagaagggcttactacccgag

SEQ ID NO: 148 - DNA, 27 bp, Artificial Sequence
  synthetic oligonucleotides
ctcagaaggcctgcctacccgaggaag

SEQ ID NO: 149 - DNA, 25 bp, Artificial Sequence
  synthetic oligonucleotides
gaaggccttacgccccgaggaaggc

SEQ ID NO: 150 - DNA, 32 bp, Artificial Sequence
  synthetic oligonucleotides
gaaggccttactacgccaggaaggccctcttg

SEQ ID NO: 151 - DNA, 40 bp, Artificial Sequence
  synthetic oligonucleotides
cagaaggccttactacccggaaaaggccctcttgtgttac

SEQ ID NO: 152 - DNA, 34 bp, Artificial Sequence
  synthetic oligonucleotides
ccttactacccgagggaagccctcttgtgttacc

SEQ ID NO: 153 - DNA, 32 bp, Artificial Sequence
  synthetic oligonucleotides
ggctactttgaaaacgagaaaaagtgccatgc

SEQ ID NO: 154 - DNA, 32 bp, Artificial Sequence
  synthetic oligonucleotides
ctactttgaaaacaaggaaaagtgccatgcgg

SEQ ID NO: 155 - DNA, 40 bp, Artificial Sequence
  synthetic oligonucleotides
ctactttgaaaacaagaaagagtgccatgcggaaatttgc

SEQ ID NO: 156 - DNA, 19 bp, Artificial Sequence
  synthetic oligonucleotides
catcggctcgtataatgtg

SEQ ID NO: 157 - DNA, 32 bp, Artificial Sequence
  synthetic oligonucleotides
gaccatctgaacctggagatcttcgcctcccg

SEQ ID NO: 158 - DNA, 32 bp, Artificial Sequence
  synthetic oligonucleotides
cgcatcttcgcctccgagctgtactaccactg

SEQ ID NO: 159 - DNA, 28 bp, Artificial Sequence
  synthetic oligonucleotides
cttcgcctcccgcgcatactaccactgg

SEQ ID NO: 160 - DNA, 26 bp, Artificial Sequence
  synthetic oligonucleotides
cctcccgcctggcttaccactggtgc

SEQ ID NO: 161 - DNA, 26 bp, Artificial Sequence
  synthetic oligonucleotides
cccgcctgtacgcgcactggtgcaag

SEQ ID NO: 162 - DNA, 25 bp, Synthliboramphus antiquus
cgcctgtactacgcctggtgcaagc

SEQ ID NO: 163 - DNA, 28 bp, Artificial Sequence
  synthetic oligonucleotides
cctgtactaccacgcatgcaagccccag

SEQ ID NO: 164 - DNA, 28 bp, Artificial Sequence
  synthetic oligonucleotides
gtactaccactgggcaaagccccagcag

SEQ ID NO: 165 - DNA, 28 bp, Artificial Sequence
  synthetic oligonucleotides
ctaccactggtgcgaaccccagcaggac

SEQ ID NO: 166 - DNA, 30 bp, Artificial Sequence
  synthetic oligonucleotides
ccttcaacccctatgagatgttagaggagc

SEQ ID NO: 167 - DNA, 40 bp, Artificial Sequence
  synthetic oligonucleotides
gatgttagaggagctagatgaaaacagtcgagccataaag

SEQ ID NO: 168 - DNA, 35 bp, Artificial Sequence
  synthetic oligonucleotides
gctagataaaaacagtgaagccataaagcgacggc

SEQ ID NO: 169 - DNA, 39 bp, Artificial Sequence
  synthetic oligonucleotides
ctagataaaaacagtcgagaaataaagcgacggcttgac

SEQ ID NO: 170 - DNA, 38 bp, Artificial Sequence
  synthetic oligonucleotides
gataaaaacagtcgagccgcaaagcgacggcttgacag

SEQ ID NO: 171 - DNA, 38 bp, Artificial Sequence
  synthetic oligonucleotides
gataaaaacagtcgagccgaaaagcgacggcttgacag

SEQ ID NO: 172 - DNA, 45 bp, Artificial Sequence
  synthetic oligonucleotides
gataaaaacagtcgagccatagaacgacggcttgacaggataaag

SEQ ID NO: 173 - DNA, 25 bp, Artificial Sequence
  synthetic oligonucleotides
gagccataaaggaacggcttgacag

SEQ ID NO: 174 - DNA, 38 bp, Artificial Sequence
  synthetic oligonucleotides
cagtcgagccataaaggaggagcttgacaggataaagc

SEQ ID NO: 175 - DNA, 35 bp, Artificial Sequence
  synthetic oligonucleotides
gtcgagccataaagcgagagcttgacaggataaag

SEQ ID NO: 176 - DNA, 31 bp, Artificial Sequence
  synthetic oligonucleotides
gccataaagcgacgggctgacaggataaagc

SEQ ID NO: 177 - DNA, 37 bp, Artificial Sequence
  synthetic oligonucleotides
gagccataaagcgacgggaagacaggataaagcagtc

SEQ ID NO: 178 - DNA, 39 bp, Artificial Sequence
  synthetic oligonucleotides
cataaagcgacggcttgacgagataaagcagtcctaatc

SEQ ID NO: 179 - DNA, 32 bp, Artificial Sequence
  synthetic oligonucleotides
cggcttgacaggatagagcagtcctaatctag

SEQ ID NO: 180 - DNA, 44 bp, Artificial Sequence; identical to SEQ ID NO: 141
  synthetic oligonucleotides

SEQ ID NO: 181 - DNA, 26 bp, Artificial Sequence
  synthetic oligonucleotides
cagtttaacaacgagcgccgcctcag

SEQ ID NO: 182 - DNA, 40 bp, Artificial Sequence; identical to SEQ ID NO: 143
  synthetic oligonucleotides

SEQ ID NO: 183 - DNA, 39 bp, Artificial Sequence; identical to SEQ ID NO: 144
  synthetic oligonucleotides

SEQ ID NO: 184 - DNA, 28 bp, Artificial Sequence
  synthetic oligonucleotides
caagcgccgcctcgaaaggccttactac

SEQ ID NO: 185 - DNA, 40 bp, Artificial Sequence; identical to SEQ ID NO: 146
  synthetic oligonucleotides

SEQ ID NO: 186 - DNA, 26 bp, Artificial Sequence
  synthetic oligonucleotides
cgcctcagaagggcttactacccgag

SEQ ID NO: 187 - DNA, 27 bp, Artificial Sequence
  synthetic oligonucleotides
ctcagaaggcctgcctacccgaggaag

SEQ ID NO: 188 - DNA, 25 bp, Artificial Sequence
  synthetic oligonucleotides
gaaggccttacgccccgaggaaggc

SEQ ID NO: 189 - DNA, 32 bp, Artificial Sequence; identical to SEQ ID NO: 150
  synthetic oligonucleotides

SEQ ID NO: 190 - DNA, 40 bp, Artificial Sequence; identical to SEQ ID NO: 151
  synthetic oligonucleotides

SEQ ID NO: 191 - DNA, 34 bp, Artificial Sequence; identical to SEQ ID NO: 152
  synthetic oligonucleotides

SEQ ID NO: 192 - DNA, 32 bp, Artificial Sequence; identical to SEQ ID NO: 153
  synthetic oligonucleotides

SEQ ID NO: 193 - DNA, 44 bp, Artificial Sequence
  synthetic oligonucleotides
gtctcctttcatctcaacaggaaaagtgccatgcggaaatttgc

SEQ ID NO: 194 - DNA, 40 bp, Artificial Sequence; identical to SEQ ID NO: 155
  synthetic oligonucleotides

SEQ ID NO: 195 - DNA, 19 bp, Artificial Sequence
  synthetic oligonucleotides
catcggctcgtataatgtg

SEQ ID NO: 196 - DNA, 32 bp, Artificial Sequence; identical to SEQ ID NO: 158
  synthetic oligonucleotides

SEQ ID NO: 197 - DNA, 28 bp, Artificial Sequence
  synthetic oligonucleotides
cttcgcctcccgcgcatactaccactgg

SEQ ID NO: 198 - DNA, 26 bp, Artificial Sequence
  synthetic oligonucleotides
cctcccgcctggcttaccactggtgc

SEQ ID NO: 199 - DNA, 26 bp, Artificial Sequence
  synthetic oligonucleotides
cccgcctgtacgcgcactggtgcaag

SEQ ID NO: 200 - DNA, 25 bp, Artificial Sequence
  synthetic oligonucleotides
cgcctgtactacgcctggtgcaagc

SEQ ID NO: 201 - DNA, 28 bp, Artificial Sequence
  synthetic oligonucleotides
cctgtactaccacgcatgcaagccccag

SEQ ID NO: 202 - DNA, 28 bp, Artificial Sequence
  synthetic oligonucleotides
gtactaccactgggcaaagccccagcag

SEQ ID NO: 203 - DNA, 28 bp, Artificial Sequence
  synthetic oligonucleotides
ctaccactggtgcgaaccccagcaggac

SEQ ID NO: 204 - DNA, 30 bp, Artificial Sequence; identical to SEQ ID NO: 166
  synthetic oligonucleotides

SEQ ID NO: 205 - DNA, 40 bp, Artificial Sequence; identical to SEQ ID NO: 167
  synthetic oligonucleotides

SEQ ID NO: 206 - DNA, 35 bp, Artificial Sequence; identical to SEQ ID NO: 168
  synthetic oligonucleotides

SEQ ID NO: 207 - DNA, 39 bp, Artificial Sequence; identical to SEQ ID NO: 169
  synthetic oligonucleotides

SEQ ID NO: 208 - DNA, 38 bp, Artificial Sequence; identical to SEQ ID NO: 170
  synthetic oligonucleotides

SEQ ID NO: 209 - DNA, 38 bp, Artificial Sequence; identical to SEQ ID NO: 171
  synthetic oligonucleotides

SEQ ID NO: 210 - DNA, 45 bp, Artificial Sequence; identical to SEQ ID NO: 172
  synthetic oligonucleotides

SEQ ID NO: 211 - DNA, 25 bp, Artificial Sequence
  synthetic oligonucleotides
gagccataaaggaacggcttgacag

SEQ ID NO: 212 - DNA, 38 bp, Artificial Sequence; identical to SEQ ID NO: 174
  synthetic oligonucleotides

SEQ ID NO: 213 - DNA, 35 bp, Artificial Sequence; identical to SEQ ID NO: 175
  synthetic oligonucleotides

SEQ ID NO: 214 - DNA, 31 bp, Artificial Sequence; identical to SEQ ID NO: 176
  synthetic oligonucleotides

SEQ ID NO: 215 - DNA, 37 bp, Artificial Sequence; identical to SEQ ID NO: 177
  synthetic oligonucleotides

SEQ ID NO: 216 - DNA, 39 bp, Artificial Sequence
  synthetic oligonucleotides
cataaagcgacggcttgacgagataaagcagtcctgagc

SEQ ID NO: 217 - DNA, 27 bp, Artificial Sequence
  synthetic oligonucleotides
gcttgacaggatagagcagtcctgagc

SEQ ID NO: 218 - DNA, 51 bp, Artificial Sequence
  synthetic oligonucleotides
agatccgcggccgcgccgccaccatgaatccacagatcagaaatccgatgg

SEQ ID NO: 219 - DNA, 52 bp, Artificial Sequence
  synthetic oligonucleotides
tgaggtcccgggagtctcgctgccgctgtttccctgattctggagaatggcc

SEQ ID NO: 220 - DNA, 51 bp, Artificial Sequence
  synthetic oligonucleotides
agatccgcggccgcgccgccaccatgaatccacagatcagaaacccgatga

SEQ ID NO: 221 - DNA, 52 bp, Artificial Sequence
  synthetic oligonucleotides
tgaggtcccgggagtctcgctgccgctctggagactctcccgtagccttctt

SEQ ID NO: 222 - DNA, 51 bp, Artificial Sequence; identical to SEQ ID NO: 218
  synthetic oligonucleotides

SEQ ID NO: 223 - DNA, 52 bp, Artificial Sequence
  synthetic oligonucleotides
tgaggtcccgggagtctcgctgccgctctggagaatctcccgtagccttctt

SEQ ID NO: 224 - DNA, 51 bp, Artificial Sequece
  synthetic oligonucleotides
agatccgcggccgcgccgccaccatgaagcctcacttcagaaacacagtgg

SEQ ID NO: 225 - DNA, 52 bp, Artificial Sequence
  synthetic oligonucleotides
tgaggtcccgggagtctcgctgccgctctcgagaatctcctgcagcttgctg

SEQ ID NO: 226 - DNA, 51 bp, Artificial Sequence; identical to SEQ ID NO: 224
  synthetic oligonucleotides

SEQ ID NO: 227 - DNA, 52 bp, Artificial Sequence
  synthetic oligonucleotides
tgaggtcccgggagtctcgctgccgctgttttcctgattctggagaatggcc

SEQ ID NO: 228 - DNA, 52 bp, Artificial Sequence
  synthetic oligonucleotides
agatccgcggccgcgccgccaccatggctctgttaacagccgaaacattccg

SEQ ID NO: 229 - DNA, 52 bp, Artificial Sequence
  synthetic oligonucleotides
tgaggtcccgggagtctcgctgccgcttcaggactgctttatcctgtcaagc

SEQ ID NO: 230 - DNA, 38 bp, Artificial Sequence
  synthetic oligonucleotides
ccatgcccgaaggtcacgtacaggagcggaccatcttc

SEQ ID NO: 231 - DNA, 38 bp, Artificial Sequence
  synthetic oligonucleotides
gaagatggtccgctcctgtacgtgaccttcgggcatgg

SEQ ID NO: 232 - DNA, 30 bp, Artificial Sequence
  synthetic oligonucleotides
ggacggcaacatttcagggcacaagctgga

SEQ ID NO: 233 - DNA, 30 bp, Artificial Sequence
  synthetic oligonucleotides
tccagcttgtgccctgaaatgttgccgtcc

SEQ ID NO: 234 - DNA, 35 bp, Artificial Sequence
  synthetic oligonucleotides
cgacaaccactattcaagtacccagtcggccctga

SEQ ID NO: 235 - DNA, 35 bp, Artificial Sequence
  synthetic oligonucleotides
tcagggccgactgggtacttgaatagtggttgtcg

SEQ ID NO: 236 - DNA, 25 bp, Artificial Sequence
  synthetic oligonucleotides
acaccccgaaggtcacgtacaggag

SEQ ID NO: 237 - DNA, 26 bp, Artificial Sequence
  synthetic oligonucleotides
aaaacctcctgtacgtgaccttcggg

SEQ ID NO: 238 - DNA, 26 bp, Artificial Sequence
  synthetic oligonucleotides
acacccaacatttcagggcacaagcg

SEQ ID NO: 239 - DNA, 26 bp, Artificial Sequence
  synthetic oligonucleotides
aaaacgcttgtgccctgaaatgttgg

SEQ ID NO: 240 - DNA, 26 bp, Artificial Sequence
  synthetic oligonucleotides
acaccccactattcaagtacccagtg

SEQ ID NO: 241 - DNA, 26 bp, Artificial Sequence
  synthetic oligonucleotides
aaaacactgggtacttgaatagtggg

SEQ ID NO: 242 - DNA, 23 bp, Artificial Sequence
  synthetic oligonucleotides
cgactcactatagggagagcggc

SEQ ID NO: 243 - DNA, 24 bp, Artificial Sequence
  synthetic oligonucleotides
aagaacatcgattttccatggcag

SEQ ID NO: 244 - DNA, 53 bp, Artificial Sequence
  synthetic oligonucleotides
acactctttccctacacgacgctcttccgatcttcgagctgaagggcatcgac

SEQ ID NO: 245 - DNA, 54 bp, Artificial Sequence
  synthetic oligonucleotides
acactctttccctacacgacgctcttccgatctctcgagctgaagggcatcgac

SEQ ID NO: 246 - DNA, 55 bp, Artificial Sequence
  synthetic oligonucleotides
acactctttccctacacgacgctcttccgatctcctcgagctgaagggcatcgac

SEQ ID NO: 247 - DNA, 54 bp, Artificial Sequence
  synthetic oligonucleotides
gtgactggagttcagacgtgtgctcttccgatcttagacgttgtggctgttgta

SEQ ID NO: 248 - DNA, 55 bp, Artificial Sequence
  synthetic oligonucleotides
gtgactggagttcagacgtgtgctcttccgatctgtagacgttgtggctgttgta

SEQ ID NO: 249 - DNA, 56 bp, Artificial Sequence
  synthetic oligonucleotides
gtgactggagttcagacgtgtgctcttccgatctggtagacgttgtggctgttgta

SEQ ID NO: 250 - DNA, 53 bp, Artificial Sequence
  synthetic oligonucleotides
acactctttccctacacgacgctcttccgatctccatcggcgacggccccgtg

SEQ ID NO: 251 - DNA, 54 bp, Artificial Sequence
  synthetic oligonucleotides
acactctttccctacacgacgctcttccgatctcccatcggcgacggccccgtg

SEQ ID NO: 252 - DNA, 55 bp, Artificial Sequence
  synthetic oligonucleotides
acactctttccctacacgacgctcttccgatctccccatcggcgacggccccgtg

SEQ ID NO: 253 - DNA, 54 bp, Artificial Sequence
  synthetic oligonucleotides
gtgactggagttcagacgtgtgctcttccgatctcgcttctcgttggggtcttt

SEQ ID NO: 254 - DNA, 55 bp, Artificial Sequence
  synthetic oligonucleotides
gtgactggagttcagacgtgtgctcttccgatctgcgcttctcgttggggtcttt

SEQ ID NO: 255 - DNA, 56 bp, Artificial Sequence
  synthetic oligonucleotides
gtgactggagttcagacgtgtgctcttccgatctggcgcttctcgttggggtcttt

SEQ ID NO: 256 - DNA, 93 bp, Artificial Sequence
  synthetic oligonucleotide
accgcatcgagctgaagggcatcgacttcaaggaggacggcaacacaagctggagtacaactacaacagccacaacgtctatatcatggccga

(19) United States
(12) Patent Application Publication
(10) Pub. No.: US 2020/0131496 A1
(43) Pub. Date: Apr. 30, 2020

(54) APOBEC ENZYMES WITH INCREASED DNA EDITING ACTIVITY, AND METHODS FOR THEIR USE

(71) Applicant: Regents of the University of Minnesota, Minneapolis, MN (US)

(72) Inventors: Reuben S. Harris, St. Paul, MN (US); Amber Renee St. Martin, Maple Grove, MN (US); Nadine Shaban, St. Louis Park, MN (US); Daniel James Salamango, Crystal, MN (US)

(21) Appl. No.: 16/663,578

(22) Filed: Oct. 25, 2019

Related U.S. Application Data
(60) Provisional application No. 62/751,348, filed on Oct. 26, 2018.

(51) Int. Cl. C12N 9/78; C12N 9/22

(52) U.S. Cl. CPC C12N 9/78; C12N 2800/80; C12N 9/22

(57) ABSTRACT

Materials and methods for using modified APOBEC polypeptides for modification of DNA sequences are provided herein.

### CROSS-REFERENCE TO RELATED APPLICATIONS

This application claims benefit of priority from U.S. Provisional Application No. 62/751,348, filed on Oct. 26, 2018. The disclosure of the prior application is considered part of (and is incorporated by reference in) the disclosure of this application.

### STATEMENT AS TO FEDERALLY SPONSORED RESEARCH

This invention was made with government support under AI064046, GM118000, GM118047, and CA206309, awarded by the National Institutes of Health. The government has certain rights in the invention.

### TECHNICAL FIELD

This document relates to modified APOBEC enzymes that have reduced ability to interact with RNA and increased deaminase activity, and methods for using the modified APOBEC enzymes to edit DNA sequences.

### BACKGROUND

Vertebrates encode variable numbers of active polynucleotide cytosine deaminase enzymes that collectively are called apolipoprotein B mRNA-editing complex (APOBEC) proteins (Conticello, Genome Biol 9:229, 2008; and Harris and Dudley, Virology 479-480C:131-145, 2015). These enzymes catalyze hydrolytic deamination of cytidine or deoxycytidine in polynucleotides to uridine or deoxyuridine, respectively. All vertebrate species have activation-induced deaminase (AID), which is essential for antibody gene diversification through somatic hypermutation and class switch recombination (Di Noia and Neuberger, Annu Rev Biochem 76:1-22, 2007; and Robbiani and Nussenzweig, Annu Rev Pathol 8:79-103, 2013). Most vertebrates also have APOBEC 1, which edits cytosine nucleobases in RNA and single-stranded DNA (ssDNA), and functions in regulating the transcriptome and likely also in blocking the spread of endogenous and exogenous mobile elements such as viruses (Fossat and Tam, RNA Biol 11:1233-1237, 2014; and Koito and Ikeda, Front Microbiol 4:28, 2013). The APOBEC3 subfamily of enzymes is specific to mammals, is subject to extreme copy number variation, elicits strong preferences for ssDNA, and provides innate immune protection against a wide variety of DNA-based parasites, including common retrotransposons L1 and Alu, and retroviruses such as HIV-1 (Harris and Dudley, supra; Malim and Bieniasz, Cold Spring Harb Perspect Med 2:a006940, 2012; and Simon et al., Nat Immunol 16:546-553, 2015). Human cells have the potential to produce up to seven distinct APOBEC3 enzymes, APOBEC3A through APOBEC3H (A3A-A3H, excluding A3E), although most cells express subsets due to differential gene regulation (Refsland et al., Nucleic Acids Res 38:4274-4284, 2010; Koning et al., J Virol 83:9474-9485, 2009; Stenglein et al., Nat Struct Mol Biol 17:222-229, 2010; and Burns et al., Nature 494:366-370, 2013a).

### SUMMARY

This document is based, at least in part, on the discovery that APOBEC enzymes bind RNA, and that APOBEC3H is regulated by a RNA-mediated dimerization mechanism. This discovery has allowed for the design of variant APOBEC molecules that, together with a targeting system such as a Clustered Regularly Interspersed Short Palindromic Repeats/CRISPR-associated (CRISPR/Cas) system, can be used to modify DNA in a specific, targeted fashion with increased efficiency and activity as compared to systems that do not include such modified APOBEC molecules.

In one aspect, this document features a variant APOBEC polypeptide containing an amino acid sequence having one or more amino acid substitutions as compared to a reference APOBEC amino acid sequence, wherein the variant APOBEC polypeptide has reduced RNA binding, reduced oligomerization, or reduced RNA binding and reduced oligomerization, as compared to a polypeptide having the reference APOBEC amino acid sequence. The polypeptide can have one, two, three, four, or five amino acid substitutions as compared to the reference APOBEC amino acid sequence. The polypeptide can be an APOBEC3H (A3H) polypeptide, and the reference APOBEC amino acid sequence can be set forth in SEQ ID NO:81. The variant APOBEC polypeptide can have increased DNA deaminase activity as compared to the reference APOBEC polypeptide having the sequence set forth in SEQ ID NO:81. The one or more amino acid substitutions can be at one or more of positions corresponding to positions 18, 20, 114, 115, 171, 172, 173, 175, 176, and 179 of SEQ ID NO:81. The variant polypeptide can further contain a substitution at position 52 of SEQ ID NO:81. The variant APOBEC polypeptide can have a glutamic acid substituted at position 18, a glutamic acid substituted at position 20, an alanine substituted at position 114, an alanine substituted at position 115, a glutamic acid substituted at position 171, a glutamic acid substituted at position 172, a glutamic acid or an alanine substituted at position 173, a glutamic acid substituted at position 175, a glutamic acid substituted at position 176, a glutamic acid substituted at position 179, or up to five combinations thereof. For example, the variant APOBEC polypeptide can have a glutamic acid substituted at position 175, a glutamic acid substituted at position 176, or a glutamic acid substituted at position 175 and a glutamic acid substituted at position 176. The variant APOBEC polypeptide can further have a glutamic acid substituted at position 52.

In another aspect, this document features a fusion polypeptide containing (a) a first portion that includes a variant APOBEC polypeptide having an amino acid sequence with one or more amino acid substitutions as compared to a reference APOBEC amino acid sequence, wherein the variant APOBEC polypeptide has reduced RNA binding, reduced oligomerization, or reduced RNA binding and reduced oligomerization, as compared to a polypeptide having the reference APOBEC amino acid sequence; and (b) a second portion that includes a Cas9 polypeptide having the ability to complex with a guide RNA (gRNA), but lacking nuclease activity. The variant APOBEC polypeptide can have one, two, three, four, or five amino acid substitutions as compared to the reference APOBEC amino acid sequence. The variant APOBEC polypeptide can be fused to the N-terminus of the Cas9 polypeptide or to the C-terminus of the Cas9 polypeptide. The variant APOBEC polypeptide can be coupled to the Cas9 polypeptide via a linker. The fusion polypeptide can contain a third portion that includes an additional functional domain from an inhibitor of uracil DNA glycosylase. The variant APOBEC polypeptide can be an A3H polypeptide, and the reference APOBEC amino acid sequence can be set forth in SEQ ID NO:81. The variant APOBEC polypeptide can have increased DNA deaminase activity as compared to the reference APOBEC polypeptide having the sequence set forth in SEQ ID NO:81. The one or more amino acid substitutions within the variant APOBEC polypeptide can be at one or more of the positions corresponding to positions 18, 20, 114, 115, 171, 172, 173, 175, 176, and 179 of SEQ ID NO:81. The variant APOBEC polypeptide can further include a substitution at position 52 of SEQ ID NO:81. The variant APOBEC polypeptide can have a glutamic acid substituted at position 18, a glutamic acid substituted at position 20, an alanine substituted at position 114, an alanine substituted at position 115, a glutamic acid substituted at position 171, a glutamic acid substituted at position 172, a glutamic acid or an alanine substituted at position 173, a glutamic acid substituted at position 175, a glutamic acid substituted at position 176, a glutamic acid substituted at position 179, or up to five combinations thereof. For example, the variant APOBEC polypeptide can have a glutamic acid substituted at position 175, a glutamic acid substituted at position 176, or a glutamic acid substituted at position 175 and a glutamic acid substituted at position 176. The variant APOBEC polypeptide can further include a glutamic acid substituted at position 52.

In another aspect, this document also features a nucleic acid molecule encoding a variant APOBEC polypeptide as described herein. This document also features a nucleic acid molecule encoding a fusion polypeptide as described herein. In some cases, a nucleic acid molecule encoding a fusion polypeptide also can include (i) a sequence encoding a Clustered Regularly Interspersed Short Palindromic Repeats RNA (crRNA) and a sequence encoding a trans-activating crRNA (tracrRNA), or (ii) a sequence encoding a gRNA.

In another aspect, this document features a method for targeted modification of a selected DNA sequence. The method can include contacting the DNA sequence with (a) a fusion polypeptide that has (i) a first portion containing a variant APOBEC polypeptide that includes an amino acid sequence having one or more amino acid substitutions as compared to a reference APOBEC amino acid sequence, where the variant APOBEC polypeptide has reduced RNA binding, reduced oligomerization, or reduced RNA binding and reduced oligomerization, as compared to a polypeptide having the reference APOBEC amino acid sequence, and (ii) a second portion containing a Cas9 polypeptide having the ability to complex with a crRNA or a gRNA, but lacking nuclease activity; and (b) a nucleic acid containing a crRNA sequence and a tracrRNA sequence targeted to the selected sequence, or containing a gRNA sequence, such that the nucleic acid complexes with the fusion polypeptide and directs the fusion polypeptide to the selected sequence, where the method includes contacting the DNA sequence with the fusion polypeptide and the nucleic acid in an amount effective for deamination of a deoxycytidine within the selected DNA sequence. The variant APOBEC polypeptide can have one, two, three, four, or five amino acid substitutions as compared to the reference APOBEC amino acid sequence. The fusion polypeptide can include a third portion that contains an additional functional domain from an inhibitor of uracil DNA glycosylase. The variant APOBEC polypeptide can be an A3H polypeptide, and the reference APOBEC amino acid sequence can be set forth in SEQ ID NO:81. The variant APOBEC polypeptide can have increased DNA deaminase activity as compared to the reference APOBEC polypeptide having the sequence set forth in SEQ ID NO:81. The one or more amino acid substitutions within the variant APOBEC polypeptide can be at one or more of the positions corresponding to positions 18, 20, 114, 115, 171, 172, 173, 175, 176, and 179 of SEQ ID NO:81. The variant APOBEC polypeptide can further include a substitution at position 52 of SEQ ID NO:81. The variant APOBEC polypeptide can have a glutamic acid substituted at position 18, a glutamic acid substituted at position 20, an alanine substituted at position 114, an alanine substituted at position 115, a glutamic acid substituted at position 171, a glutamic acid substituted at position 172, a glutamic acid or an alanine substituted at position 173, a glutamic acid substituted at position 175, a glutamic acid substituted at position 176, a glutamic acid substituted at position 179, or up to five combinations thereof. For example, the variant APOBEC polypeptide can have a glutamic acid substituted at position 175, a glutamic acid substituted at position 176, or a glutamic acid substituted at position 175 and a glutamic acid substituted at position 176. The variant APOBEC polypeptide can further have a glutamic acid substituted at position 52. The introducing can include introducing into the cell a nucleic acid encoding the fusion polypeptide, and the method can further include maintaining the cell under conditions in which the nucleic acid encoding the fusion polypeptide is expressed. The nucleic acid encoding the fusion polypeptide and the nucleic acid containing (i) the crRNA sequence and the tracrRNA sequence or (ii) the gRNA sequence can be in a single vector, or can be in separate vectors. The selected DNA sequence can be associated with a clinical condition, and deamination of the cytidine can result in a sequence that is not associated with the clinical condition. The contacting can be in vitro or in vivo. For example, the contacting can be in a mammal identified as having a clinical condition.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C show that RNase A digestion enabled APOBEC3H purification and DNA deaminase activity. FIG. 1A is an image of a Coomassie-stained gel showing His6-SUMO-A3H recovery from E. coli±RNase A treatment. FIG. 1B is a graph plotting DNA deaminase activity of His6-SUMO-A3H in extracts from E. coli±RNase A treatment (mean±SD; n=3 experiments). The inset gel image shows A3H-mediated conversion of a single-stranded DNA substrate to product, S to P, in the presence of RNase A). Lysate units (μl volumes) were chosen to include reactions with single-hit kinetics. FIG. 1C is a graph plotting DNA deaminase activity of untagged A3H in extracts from 293T cells, with experimental parameters analogous to those in FIG. 1B.

FIGS. 2A and 2B show that an initial structural model predicted two positively charged patches in human APOBEC3H. FIG. 2A shows an initial structural model of human A3H. A single zinc ion in the active site, loops 1, 3, and 7, and the α6-helix are labeled for orientation. FIG. 2A also includes space-filled schematics showing the predicted electrostatic potential for A3H in the same orientation (center) and rotated 120° (right) compared to the initial model. Patch 1 encompasses the active site and residues known to be required for DNA deamination. Patch 2 is distinct and includes several residues in the α6-helix. FIG. 2B shows an alignment of amino acid sequences from human A3H, A3A, and A3B C-terminal domain (ctd) for the loop 1, loop 3, loop 7, and α6-helix regions, as indicated. Residue numbers correspond to the 183 amino acid splice variant of human A3H haplotype II, and underlined residues were changed by site-directed mutagenesis.

FIGS. 3A-3C show the identification of hyperactive human APOBEC3H mutants. FIG. 3A is a graph plotting Rif^{R} (rpoB) mutation frequency of E. coli expressing the indicated His6-Sumo-A3H constructs (WT, wild-type). Each dot represents data from an individual culture (n>5 per condition) with short horizontal lines depicting medians. The lower dashed line represents the background mutation frequency of E. coli expressing an A3H catalytic mutant (E56A). The upper dashed line shows the median mutation frequency of E. coli expressing WT A3H. FIG. 3B is a graph plotting data from a representative Rif^{R} mutation experiment comparing WT A3H and the indicated mutants, including five hypermutators. Conditions and labels are analogous to those for FIG. 3A. FIG. 3C is a graph plotting quantification of key Rif^{R} mutation data. Each histogram bar reports the mean±SEM of the median mutation frequency from three independent experiments (FIG. 3B and two additional independent experiments). The mutation frequencies induced by the five hypermutators were significantly greater than that of WT A3H (p<0.01 by Student's t-test).

FIGS. 4A and 4B show DNA deaminase activity of APOBEC3H and derivatives in 293T extracts. The DNA deaminase activities of untagged wild-type (WT) human A3H or the indicated amino acid substitution mutants were compared in 293T extracts without (FIG. 4A) or with (FIG. 4B) RNase A treatment (S, substrate; P, product). Corresponding immunoblots show levels of A3H in cell lysates using a murine mAb (P1D8) or a rabbit pAb (Novus), as indicated. β-actin was used as a loading control.

FIGS. 5A-5D indicate the structure of a human APOBEC3H dimer bound to a RNA double helix. FIG. 5A is an image of a Coomassie-stained gel showing recovery of mCherry-A3H-K52E (a fully functional A3H derivative) from E. coli. The asterisks represent the position of heat-induced mCherry degradation products (Gross et al., Proc Natl Acad Sci USA 97:11990-11995, 2000). FIG. 5B is a graph showing size-exclusion chromatography (SEC) profiles for wild-type mCherry-A3H (solid line), mCherry-A3H-K52E (line 1), mCherry-A3H-E56A (line 2), mCherry-A3H-E56A-K52E (line 3), and a representative RNA binding-defective mutant (W115A/R175/6E with E56A to prevent E. coli toxicity; dashed line). Molecular weight standards are shown on the dashed line above chromatograms. FIG. 5C is an image of a polyacrylamide-urea gel of A3H preparations after proteinase K treatment. Wild-type, K52E, E56A, and E56A-K52E preparations contained multiple small RNA species that were sensitive to RNase A but not DNase I. A representative RNA binding defective mutant (W115A/R175/6E with E56A to prevent E. coli toxicity) lacked RNA. FIG. 5D shows the X-ray structure of human A3H in complex with duplex RNA. Left: a crystallographic pose showing the A3H electrostatic surface potential and double-stranded RNA sandwiched between the positively charged surfaces (patch 2) of two A3H monomers. A partially transparent zoom-in of the duplex RNA-binding region highlights positively charged residues in loop 1 (L1 R18) and α6-helix (K168, R171, A172, R175, R176, and R179). A 180° rotation (right) shows the back side of the positively charged cradle bound to double-stranded RNA (loop 7, L7, labeled for orientation). This orientation also shows the active site pocket (patch 1, asterisked). The zoom-in depicts the observed loop 1 contacts between the two A3H monomers.

FIGS. 6A and 6B are electron density maps for the APOBEC3H-duplex RNA crystal structure. The experimental electron density maps, obtained using a SAD phasing method from zinc anomalous signal and contoured at 1σ (mesh), revealed an A-form RNA duplex in the mCherry-A3H-K52E crystal structure (pdb 6BOB). Density was clearly visible for double helical-RNA in an A-form structure. The final refined RNA has a C3′-endo conformation for each ribose sugar pucker. The final refined RNA also had an average X-displacement of −5.5 Å, an average rise between adjacent base pairs of 2.86 Å, and an average twist angle between adjacent base pairs of 29.3°, respectively.

FIG. 7 shows the structure of human APOBEC3H and comparisons with related APOBEC family members. Top row: Ribbon schematic of an A3H-K52E monomer alone (pdb 6BOB) and in comparison by superposition with human A3A (pdb 5SWW) and human A3C (pdb 3VOW). Notable differences include a longer loop 1 in A3H, a shorter loop 3 in A3H, and an amino terminal 12-residue extension in A3A that did not resolve structurally. Middle row: Electrostatic potential of A3H, A3A, and A3C in the same orientation as the top row, highlighting the conserved zinc-coordinating active site region (patch 1 in A3H). Bottom row: 120° rotation of structures in the middle row, highlighting the RNA binding domain of A3H (patch 2) and a homologous basic patch in A3C. In contrast, A3A has a more negatively charged α6-helix region.

FIGS. 8A and 8B show superposition of two independent human APOBEC3H crystal structures. FIG. 8A is a ribbon schematic of an A3H-K52E monomer bound to duplex RNA (pdb 6BOB). Active site zinc-coordinating residues include His54, Glu56, Cys85, and Cys88. FIG. 8B is a superposition of ribbon schematics of crystal structures of A3H-K52E (gray; pdb 6BOB) and the catalytic mutant derivative A3H-K52E-E56A (white; pdb 6BBO) showing identical duplex RNA binding mechanisms.

FIGS. 9A-9C show that RNA-binding is required for human APOBEC3H cytoplasmic localization. FIG. 9A includes representative images of untagged A3H (WT) and mutant derivatives (E56A, R18E, H114A, W115A, A172E, R175/6E, R179E) in 293T cells and HeLa cells (top and bottom panels, respectively). A3B nuclear localization and A3G cytoplasmic localization provided positive controls, and the no APOBEC reaction provided a negative control. DAPI staining was used to define the nuclear compartment (10 μm scale bar). Quantification of the ratio of nuclear/cytoplasmic staining of the indicated constructs in 293T and HeLa cells are shown in FIG. 9B and FIG. 9C, respectively (n=25 cells represented by individual dots with mean±SEM superimposed; ns, not significant; **, p<0.001; ***, p<0.0001 by 2-sided Student's t-test; Nuc, nuclear; WC, whole cell; Cyto, cytoplasmic).

FIGS. 10A-10C also show that RNA binding is required for human APOBEC3H cytoplasmic localization. FIG. 10A includes representative images of mCherry-A3H (WT) and mutant derivatives (E56A, R18E, H114A, W115A, A172E, R175/6E) in 293T cells (top) and HeLa cells (bottom). The no plasmid DNA control showed low background fluorescence. DAPI staining defines the nuclear compartment (10 μm scale bar). FIGS. 10B and 10C are graphs plotting the quantified ratio of nuclear/cytoplasmic staining of the indicated constructs in 293T (FIG. 10B) and HeLa (FIG. 10C) cells (n=25 cells represented by individual dots with mean±SEM superimposed; ns, not significant; **, p<0.001; ***, p<0.0001 by 2-sided Student's t-test; Nuc, nuclear; WC, whole cell; Cyto, cytoplasmic).

FIG. 11 is a pair of graphs and a series of immunoblots showing that RNA binding is required for HIV-1 restriction by human APOBEC3H. Relative levels of infectivity are plotted for Vif-null HIV-1LAI produced in 293T cells in the presence of 12.5 ng, 50 ng, 200 ng, and 400 ng of the indicated untagged, human A3H expression constructs (WT, wild-type; average±SD of three biologically independent experiments; average values for each condition normalized to those of the no A3 control). Corresponding immunoblots are shown for proteins in viral particles and virus-producing 293T cells, as indicated.

FIG. 12 includes a pair of graphs (top) and images of immunoblots (bottom) demonstrating that RNA binding is required for HIV-1 restriction by human APOBEC3H. Relative levels of infectivity are plotted for Vif-null HIV-1IIIB produced in the presence of 12.5 ng, 50 ng, 200 ng, and 400 ng of the indicated untagged, human A3H expression constructs (WT, wild-type; average±SD of three biologically independent experiments; average values±SD for each condition are presented, normalized to those of the no A3 control). Corresponding immunoblots are shown below for proteins in virus-like particles (VLPs) and virus-producing 293T cells. Quantification of the levels of A3H packaging relative to WT are provided below the immunoblots for relevant RNA binding mutants (mean±SEM from three biologically independent experiments; statistical significance determined using one-sided Student's t-test). First, viral particle A3H and p24 levels and cellular A3H and tubulin levels were quantified using Image J. Second, the relative packaging efficiency (RPE) was calculated for each condition using the following formula: RPE: (VLP A3H/p24)/(cellular A3H/TUB). Last, normalization was done by dividing the RPE value for each RNA binding mutant by the RPE value for WT A3H. The WT RPE is 1 and each mutant packages significantly less efficiently (p<0.01 using a one-sided Student's t-test).

FIG. 13 includes a graph and images of immunoblots demonstrating that RNA binding is required for HIV-1 restriction by human APOBEC3H. Single-cycle infectivity data for Vif-null HIV-1LAI produced in the presence of 200 ng of the indicated untagged, human A3H expression constructs (WT, wild-type; average±SD of three technical replicates). Corresponding immunoblots are shown below for proteins in virus-like particles (VLPs) and virus-producing 293T cells (A3H detection with mouse mAb P1D8 and rabbit pAb Novus; anti-p24 and anti-tubulin are loading controls for VLPs and cells, respectively).

FIGS. 14A-14C are amino acid alignments comparing loop and a6 regions of human APOBEC3H and other APOBEC family members. FIG. 14A is a Clustal Omega alignment of human A3H and homologous primate A3H enzymes, highlighting the loop 1, loop 3, loop 7, and α6-helix regions. Numbers correspond to the 183 amino acid splice variant of human A3H haplotype II. GENBANK® accession numbers: Human A3H: ACK77775.1; Chimpanzee A3H: NP_001136078.1; Gorilla A3H: ACJ60858.1; Orangutan A3H: XP_009232662.1; Siamang Gibbon A3H: ACJ60860.1; African Green Monkey (AGM) A3H: NP_001332866.1; Olive Baboon A3H: NP_001332865.1; Rhesus Macaque A3H:NP_001332864.1; Pig-tailed Macaque: XP_011710626.1. The number in parentheses after each sequence is the sequence identifier. FIG. 14B is a Clustal Omega alignment of human A3H and homologous mammalian A3Z3 enzymes. The format is analogous to that for FIG. 14A. GENBANK® accession numbers: Human A3H: NP_861438.3; Pig A3Z3: ACH69768.1; Cow A3Z3: NP_001333053.1; Sheep A3Z3: NP_001154853.1; Horse A3Z3: NP_001229380.1; Dog A3Z3: NP_001333059.1; Cat A3Z3: NP_001106181.2. FIG. 14C is a Clustal Omega alignment of human A3H, A3C, and AID. The format is similar to that of FIG. 14A. GENBANK® accession numbers: Human A3H: NP_861438.3; human A3C: NP_055323.2; human AID: NP_065712.1.

FIGS. 15A-15D show that targeted single base editing in DNA is inhibited by RNA binding. FIGS. 15A and 15B include graphs plotting L202 and L138 eGFP reporter quantification of the single base editing activity of wild-type APOBEC3H (haplotype II) versus a RNA binding-defective mutant (R175/6E) (n=3, average±SD) for transfected/episomal (FIG. 15A) and chromosomal (FIG. 15B) targets. Immunoblots for a representative experiment are included below each graph. FIG. 15C shows Sanger sequences of the guide RNA (gRNA) binding regions of the eGFP L202 and L138 reporters after editing by the APOBEC3H editosomes in FIG. 15A. Single base substitutions are boxed. Deletions are represented by hyphens. The number of times each sequence was recovered is shown on the right. Numbers in parentheses indicate sequence identifiers. FIG. 15D sequence logos summarizing unbiased deep sequencing data for the gRNA binding regions of the L202 and L138 reporters. The left panel reports all full-length sequences, and the right panel reports the subset with on-target editing events. Base substitution mutations occurring in at least 5% of the reads are shown in darker text. The L202 PAM mutation was likely a PCR or MiSeq artifact due to G/C richness, as it was also present in the control reactions. The pie graphs below the sequence logos show MiSeq read proportions with no mutations (white), one or more single base substitutions (gray), or indels (black; some of which were also coincident with single base substitutions).

### DETAILED DESCRIPTION

Human cells produce up to seven distinct APOBEC enzymes, which recognize particular nucleotide substrates. The nucleobase immediately 5′ of the target cytosine (position −1 relative to the target cytosine at position 0) appears to be the most important determinant of each enzyme's intrinsic substrate preference (Carpenter et al., DNA Repair (Amst) 9:579-587, 2010; Rathore et al., J Mol Biol 425:4442-4454, 2013; Kohli et al., J Biol Chem 285:40956-40964, 2010; and Wang et al., J Exp Med 207:141-153, 2010). For example, AID preferentially deaminates single-stranded DNA cytosine bases preceded by an adenine or guanine (5′-RC). A3G uniquely targets cytosine bases preceded by another cytosine (5′-CC). APOBEC1 and the remaining APOBEC3 enzymes elicit preferences for cytosine bases preceded by a thymine (5′-TC). The −1 nucleobase preference is governed largely by a loop adjacent to the active site (loop 7), such that loop exchanges can convert one enzyme's intrinsic preference into that of another. For example, A3G with loop 7 from A3A becomes a 5′-TC preferring enzyme (Rathore et al., supra), while A3A with loop 7 from A3G becomes a 5′-CC preferring enzyme. The −2 and +1 nucleobases relative to the target cytosine also are likely to be involved in local target selection but are thought to be less influential.

The APOBEC mutation signature in cancer has been defined as C-to-T and C-to-G base substitution mutations within 5′-TC dinucleotide motifs, and most commonly within 5′-TCA and 5′-TCT trinucleotide contexts (also 5′-TCG if accounting for the genomic under-representation of this motif; Burns et al., supra; and Starrett et al., Nat Commun 7:12918, 2016). The leading candidates to explain APOBEC mutagenesis in cancer are A3A (Caval et al., Nat Commun 5:5129, 2014; Chan et al., Nat Genet 47:1067-1072, 2015; and Nik-Zainal et al., Nat Genet 46:487-491, 2014), A3B (Burns et al., supra; Starrett et al., supra; and Burns et al., Nat Genet 45:977-983, 2013b), and A3H (Starrett et al., supra).

A3H is unique among APOBEC3 subfamily members for several reasons. First, the gene encoding A3H is phylogenetically distinct, shows high conservation, and invariably anchors the 3′-end of the APOBEC3 locus (LaRue et al., BMC Mol Biol 9:104, 2008; and Münk et al., Genome Biol 9:R48, 2008). The encoded enzyme exists as a single zinc-coordinating domain Z3-type deaminase in higher primates, including humans, or as a single- or double-domain enzyme (typically Z2-Z3 organization) in most other mammals, including artiodactyls, carnivores, and rodents (LaRue et al., 2008; Münk et al., 2008). Second, unlike Z1 and Z2-type deaminase genes, the A3H or Z3-type genes show no copy number variation, existing in each species as a full gene or as the 3′ half of a gene encoding a double domain deaminase. Third, despite strict copy number conservation, A3H is the most polymorphic family member in humans, with seven reported haplotypes due to variations at five amino acid positions and four reported splice variants (Harari et al., J Virol 83:295-303, 2009; OhAinle et al., Cell Host Microbe 4:249-259, 2008; and Wang et al., J Virol 85:3142-3152, 2011).

RNA plays a role in regulating the activities of A3H, as described in the Examples herein. The most stable, active, and antiviral A3H variant in humans is the 183 amino acid haplotype II enzyme, hereafter referred to as “HapII 1-183” or simply “A3H.” RNA invariably co-purified with wild-type A3H and suppressed its deaminase activity. RNase A treatment removed most, but not all, of the bound RNA and enabled strong DNA deaminase activity. Mutagenesis of a positively charged patch with the potential to bind RNA resulted in enzymes with DNA hypermutator activity. Wild-type A3H and hyperactive variants showed dimeric and monomeric size exclusion profiles, respectively, suggesting a RNA-mediated dimerization mechanism. Indeed, crystal structures of a near wild-type human A3H-mCherry protein and a catalytic mutant derivative revealed a duplex RNA-bridged enzyme dimer involving the precise amino acid residues that yield DNA deaminase hyperactivity upon mutagenesis. Experiments with these separation-of-function mutants demonstrated that the RNA binding surface is required for A3H cytoplasmic localization and for packaging into HIV-1 particles and antiviral activity. Thus, RNA serves multiple regulatory roles in A3H biology, and may also play regulatory roles in the biology of other APOBEC enzymes.

This document therefore provides variant APOBEC polypeptides and nucleic acids, including variant A3A, A3B, A3C, A3D, A3F, A3G, A3H, AID, and APOBEC1 polypeptides and nucleic acids, are provided herein. The variant polypeptides can include one or more amino acid substitutions, additions, or deletions as compared to a wild-type APOBEC reference sequence from any vertebrate (e.g., a mammal such as a human, non-human primate, bat, pig, cow, horse, sheep, dog, cat, rat, or mouse). In some cases, a variant APOBEC polypeptide can be an active deaminase domain from a double-domain APOBEC enzyme, such as the ctd of A3B, A3D, A3F, or A3G (referred to as A3Bctd, A3Dctd, A3Fctd, and A3Gctd, respectively), such that the variant includes one or more amino acid substitutions, additions, or deletions as compared to the sequence of a reference active deaminase domain from a double-domain APOBEC enzyme. In certain cases, a reference sequence can be the wild-type A3H sequence set forth in SEQ ID NO:81.

The variant APOBEC polypeptides provided herein can be, for example, splice variants, naturally occurring amino acid variants, and variants containing mutations generated in vitro, as compared to a reference APOBEC sequence. The variant APOBEC polypeptides provided herein can have reduced or no binding affinity for RNA (e.g., binding affinity that is reduced by at least 10%, at least 25%, at least 50%, at least 90%, or at least 95%, or no detectable binding affinity for RNA using the methods disclosed herein) as compared to an APOBEC polypeptide containing the reference APOBEC amino acid sequence, and can have increased DNA deaminase activity compared to an APOBEC polypeptide containing the reference APOBEC amino acid sequence. Fusion polypeptides containing a modified APOBEC portion and a DNA-targeting (e.g., Cas9) portion (referred to herein as “editosomes”) also are provided herein. As described below, such fusion polypeptides can be used for targeted DNA editing, where the APOBEC deaminase domain is directed to a target DNA sequence by a gRNA-Cas9 complex.

The term “polypeptide” as used herein refers to a compound of two or more subunit amino acids regardless of post-translational modification (e.g., phosphorylation or glycosylation). The subunits may be linked by peptide bonds or other bonds such as, for example, ester or ether bonds. The term “amino acid” refers to either natural and/or unnatural or synthetic amino acids, including D/L optical isomers.

An “isolated” or “purified” polypeptide is separated to some extent from the cellular components with which it is normally found in nature (e.g., other polypeptides, lipids, carbohydrates, and nucleic acids). A purified polypeptide can yield a single major band on a non-reducing polyacrylamide gel. A purified polypeptide can be at least about 75% pure (e.g., at least 80%, 85%, 90%, 95%, 97%, 98%, 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

Nucleic acids encoding the “hypermutator” variant APOBEC polypeptides and the DNA-targeted variant APOBEC fusion polypeptides also are provided herein. The terms “nucleic acid” and “polynucleotide” are used interchangeably, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

The polypeptides provided herein can be introduced in a cell by using a vector encoding said polypeptides for example or as polypeptides per se by using delivery vectors associated or combined with any cellular permeabilization techniques, such as sonoporation, electroporation, lipofection, or derivatives of these or other related techniques.

As used herein, “isolated,” when in reference to a nucleic acid, refers to a nucleic acid that is separated from other nucleic acids that are present in a genome, e.g., a plant genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus (e.g., a pararetrovirus, a retrovirus, lentivirus, adenovirus, or herpes virus), or the genomic DNA of a prokaryote or eukaryote. In addition, an isolated nucleic acid can include a recombinant nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by, for example, chemical synthesis or polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be obtained by mutagenesis. For example, a donor nucleic acid sequence can be mutated using standard techniques, including oligonucleotide-directed mutagenesis and site-directed mutagenesis through PCR. See, Short Protocols in Molecular Biology, Chapter 8, Green Publishing Associates and John Wiley & Sons, edited by Ausubel et al., 1992.

Recombinant nucleic acid constructs (e.g., vectors) also are provided herein. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes one or more expression control sequences, and an “expression control sequence” is a DNA sequence that controls and regulates the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The terms “regulatory region,” “control element,” and “expression control sequence” refer to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory regions that can reside within coding sequences, such as secretory signals, Nuclear Localization Sequences (NLS) and protease cleavage sites.

As used herein, “operably linked” means incorporated into a genetic construct so that expression control sequences effectively control expression of a coding sequence of interest. A coding sequence is “operably linked” and “under the control” of expression control sequences in a cell when RNA polymerase is able to transcribe the coding sequence into RNA, which if an mRNA, then can be translated into the protein encoded by the coding sequence. Thus, a regulatory region can modulate, e.g., regulate, facilitate or drive, transcription in the plant cell, plant, or plant tissue in which it is desired to express a modified target nucleic acid.

A promoter is an expression control sequence composed of a region of a DNA molecule, typically within 1000 nucleotides upstream of the point at which transcription starts (generally near the initiation site for RNA polymerase II). Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seql.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\B12seq -i c:\seql.txt -j c:\seq2.txt -p blastn -o c:\output.txt -q -1 -r 2. To compare two amino acid sequences, the options of B12seq are set as follows: -i is set to a file containing the first amino acid sequence to be compared (e.g., C:\seql.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seql.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:81), or by an articulated length (e.g., 100 consecutive nucleotides or amino acid residues from a sequence set forth in an identified sequence), followed by multiplying the resulting value by 100. For example, an amino acid sequence that has 175 matches when aligned with the sequence set forth in SEQ ID NO:81 is 95.6 percent identical to the sequence set forth in SEQ ID NO:81 (i.e., 175/183×100=93.6). It is noted that the percent sequence identity value is rounded to the nearest tenth. For example, 75.11, 75.12, 75.13, and 75.14 is rounded down to 75.1, while 75.15, 75.16, 7.17, 75.18, and 7.19 is rounded up to 7.2. It also is noted that the length value will always be an integer.

An “effective amount” of an agent (e.g., an APOBEC-Cas9 fusion polypeptide, a nucleic acid encoding such a polypeptide, or a composition containing an APOBEC-Cas9 fusion polypeptide and a gRNA directing the fusion to a specific DNA sequence) is an amount of the agent that is sufficient to elicit a desired response. For example, an effective amount of an APOBEC-Cas9 fusion polypeptide can be an amount of the polypeptide that is sufficient to induce deamination at a specific, selected target site. It is to be noted that the effective amount of an agent as provided herein can vary depending on various factors, such as, for example, the specific allele, genome, or target site to be edited, the cell or tissue being targeted, and the agent being used.

In some cases, the reference sequence is an A3H sequence. Exemplary reference A3H amino acid sequences for the 183 amino acid haplotype II enzyme are set forth in SEQ ID NOS:81 and 83 (GENBANK® accession nos. ACK77775 and NP_861438), shown below. Representative A3H nucleic acid sequences encoding the reference A3H amino acid sequences are set forth in SEQ ID NO:82 (GENBANK® accession no. NM_145699) and SEQ ID NO:84 (GENBANK® accession no. NM_181773).

Human APOBEC3H (GENBANK ® accession no. ACK77775, such as version
ACK77775.1):
(SEQ ID NO: 81)
MALLTAETFRLQFNNKRRLRRPYYPRKALLCYQLTPQNGSTPT
RGYFENKKKCHAEICFINEIKSMGLDETQCYQVTCYLTWSPCSSCAWELVDFIKA
HDHLNLRIFASRLYYHWCKPQQDGLRLLCGSQVPVEVMGFPEFADCWENFVDH
EKPLSFNPYKMLEELDKNSRAIKRRLDRIKQS.
Human APOBEC3H (GENBANK ® accession no. FJ376614, such as version
FJ376614.1):
(SEQ ID NO: 82)
ATGGCTCTGTTAACAGCCGAAACATTCCGCTTACAGTTTAACA
ACAAGCGCCGCCTCAGAAGGCCTTACTACCCGAGGAAGGCCCTCTTGTGTTA
CCAGCTGACGCCGCAGAATGGCTCCACGCCCACCAGAGGCTACTTTGAAAAC
AAGAAAAAGTGCCATGCAGAAATTTGCTTTATTAACGAGATCAAGTCCATGG
GACTGGACGAAACGCAGTGCTACCAAGTCACCTGTTACCTCACGTGGAGCCC
CTGCTCCTCCTGTGCCTGGGAGCTGGTTGACTTCATCAAGGCTCACGACCATC
TGAACCTGCGCATCTTCGCCTCCCGCCTGTACTACCACTGGTGCAAGCCCCAG
CAGGACGGGCTGCGGCTTCTGTGTGGATCCCAGGTCCCGGTGGAGGTCATGG
GCTTCCCAGAGTTTGCTGACTGCTGGGAAAACTTTGTGGACCACGAGAAACC
GCTTTCCTTCAACCCCTATAAGATGTTAGAGGAGCTAGATAAAAACAGTCGA
GCCATAAAGCGACGGCTTGACAGGATAAAGCAGTCCTGA
Human APOBEC3H (GENBANK ® accession no. NP_861438, such as version
NP_861438.3):
(SEQ ID NO: 83)
MALLTAETFRLQFNNKRRLRRPYYPRKALLCYQLTPQNGSTPTR
GYFENKKKCHAEICFINEIKSMGLDETQCYQVTCYLTWSPCSSCAWELVDFIKAH
DHLNLGIFASRLYYHWCKPQQKGLRLLCGSQVPVEVMGFPEFADCWENFVDHE
KPLSFNPYKMLEELDKNSRAIKRRLERIKQS
Human APOBEC3H (GENBANK ® accession no. NM_181773, such as version
NM_181773.4; start and stop codons are underlined):
(SEQ ID NO: 84)
TGACTTTTGGGAGAGCTGAC
CTTTTGTGACTTTTGGGAGAGCTGCCAAAAGTGAAACTTAGTGCCTCAGACA
AGCAGGGGCAAGTCTGCTAAGGAAGCTGTGGCCAGAAGCACAGATCAGAAA
CACGATGGCTCTGTTAACAGCCGAAACATTCCGCTTACAGTTTAACAACAAG
CGCCGCCTCAGAAGGCCTTACTACCCGAGGAAGGCCCTCTTGTGTTACCAGC
TGACGCCGCAGAATGGCTCCACGCCCACGAGAGGCTACTTTGAAAACAAGAA
AAAGTGCCATGCAGAAATTTGCTTTATTAACGAGATCAAGTCCATGGGACTG
GACGAAACGCAGTGCTACCAAGTCACCTGTTACCTCACGTGGAGCCCCTGCT
CCTCCTGTGCCTGGGAGCTGGTTGACTTCATCAAGGCTCACGACCATCTGAAC
CTGGGCATCTTCGCCTCCCGCCTGTACTACCACTGGTGCAAGCCCCAGCAGA
AGGGGCTGCGGCTTCTGTGTGGATCCCAGGTCCCGGTGGAGGTCATGGGCTT
CCCAGAGTTTGCTGACTGCTGGGAAAACTTTGTGGACCACGAGAAACCGCTT
TCCTTCAACCCCTATAAGATGTTAGAGGAGCTAGATAAAAACAGTCGAGCCA
TAAAGCGACGGCTTGAGAGGATAAAGCAGTCCTGAAGTGTGGATGTTTTAGA
GAATGACTTAAGAAGTTTGCAGCTTGGACCCGTATCCCACTCATTATCAAGA
AGCAACTCAAGATGACTTTCCCTGGGGCATGTCAGTTGCCTCATAGCCTGCTG
GTCCTGTAAGCAAGCACTAAGCTCCACAGTGCCAGTTCCTTGCCCCAACCTG
GCCCCATCCAAGTACAGAAGACCTTCCTTTCCTCCTTTTTCCATATTGCTTTCT
GTTCTAAGTGGGTGAATAATTTTATAATTGAAAAAATAAAGATAAAGTCTGT
AAATCCAGCCGGGTACGGTGGCTCACGCCTGTAATCCTAGCACTTTGGGAGG
CTGAGATGCTCGGCCAATAAATTTCTATTGTTTATGAAAAAAAAAAAAAAAA
AAAAAAA
Additional representative A3H reference sequences are set forth
in SEQ ID NOS: 111 to 127.
Human APOBEC3H (GENBANK ® accession no. ACK77778, such as version
ACK77778.1):
(SEQ ID NO: 111)
MALLTAETFRLQFNNKRRLRRPYYPRKALLCYQLTPQNGSTPTR
GYFENKKKCHAEICFINEIKSMGLDETQCYQVTCYLTWSPCSSCAWELVDFIKAH
DHLNLRIFASRLYYHWCKPQQDGLRLLCGSQVPVEVMGFPDSRGTCAGSLHGYI
V
Human APOBEC3H (GENBANK ® accession no. ACK77777, such as version
ACK77777.1):
(SEQ ID NO: 112)
MALLTAETFRLQFNNKRRLRRPYYPRKALLCYQLTPQNGSTPTRG
YFENKKKCHAEICFINEIKSMGLDETQCYQVTCYLTWSPCSSCAWELVDFIKAHD
HLNLRIFASRLYYHWCKPQQDGLRLLCGSQVPVEVMGFPEFADCWENFVDHEK
PLSFNPYKMLEELDKNSRAIKRRLDRIKIPGVRAQGRYMDILCDAEV
Human APOBEC3H (GENBANK ® accession no. ACK77776, such as version
ACK77776.1):
(SEQ ID NO: 113)
MALLTAETFRLQFNNKRRLRRPYYPRKALLCYQLTPQNGSTPTRG
YFENKKKCHAEICFINEIKSMGLDETQCYQVTCYLTWSPCSSCAWELVDFIKAHD
HLNLRIFASRLYYHWCKPQQDGLRLLCGSQVPVEVMGFPEFADCWENFVDHEK
PLSFNPYKMLEELDKNSRAIKRRLDRIKS
Human APOBEC3H (GENBANK ® accession no. ACK77774.1, such as
version ACK77774.1):
(SEQ ID NO: 114)
MALLTAETFRLQFNNKRRLRRPYYPRKALLCYQLTPQNGSTPTRG
YFENKKKCHAEICFINEIKSMGLDETQCYQVTCYLTWSPCSSCAWELVDFIKAHD
HLNLGIFASRLYYHWCKPQQKGLRLLCGSQVPVEVMGFPEFADCWENFVDHEK
PLSFNPYKMLEELDKNSRAIKRRLERIKIPGVRAQGRYMDILCDAEV
Human APOBEC3H (GENBANK ® accession no. ACK77773, such as version
ACK77773.1):
(SEQ ID NO: 115)
MALLTAETFRLQFNNKRRLRRPYYPRKALLCYQLTPQNGSTPTRG
YFENKKKCHAEICFINEIKSMGLDETQCYQVTCYLTWSPCSSCAWELVDFIKAHD
HLNLGIFASRLYYHWCKPQQKGLRLLCGSQVPVEVMGFPEFADCWENFVDHEK
PLSFNPYKMLEELDKNSRAIKRRLERIKS
Human APOBEC3H (GENBANK ® accession no. ACK77772, such as version
ACK77772.1):
(SEQ ID NO: 116)
MALLTAETFRLQFNNKRRLRRPYYPRKALLCYQLTPQNGSTPTRG
YFENKKKCHAEICFINEIKSMGLDETQCYQVTCYLTWSPCSSCAWELVDFIKAHD
HLNLGIFASRLYYHWCKPQQKGLRLLCGSQVPVEVMGFPEFADCWENFVDHEK
PLSFNPYKMLEELDKNSRAIKRRLERIKQS
Human APOBEC3H (GENBANK ® accession no. Q6NTF7, such as version
Q6NTF7.4):
(SEQ ID NO: 117)
MALLTAETFRLQFNNKRRLRRPYYPRKALLCYQLTPQNGSTPTRG
YFENKKKCHAEICFINEIKSMGLDETQCYQVTCYLTWSPCSSCAWELVDFIKAHD
HLNLGIFASRLYYHWCKPQQKGLRLLCGSQVPVEVMGFPEFADCWENFVDHEK
PLSFNPYKMLEELDKNSRAIKRRLERIKIPGVRAQGRYMDILCDAEV
Human APOBEC3H (GENBANK ® accession no. NP_001159476, such as
version NP_001159476.2):
(SEQ ID NO: 118)
MALLTAETFRLQFNNKRRLRRPYYPRKALLCYQLTPQ
NGSTPTRGYFENKKKCHAEICFINEIKSMGLDETQCYQVTCYLTWSPCSSCAWEL
VDFIKAHDHLNLGIFASRLYYHWCKPQQKGLRLLCGSQVPVEVMGFPDSRGTCA
GSLHGYIV
Human APOBEC3H (GENBANK ® accession no. NP_001159474, such as
version NP_001159474.2):
(SEQ ID NO: 119)
MALLTAETFRLQFNNKRRLRRPYYPRKALLCYQLTPQ
NGSTPTRGYFENKKKCHAEICFINEIKSMGLDETQCYQVTCYLTWSPCSSCAWEL
VDFIKAHDHLNLGIFASRLYYHWCKPQQKGLRLLCGSQVPVEVMGFPEFADCW
ENFVDHEKPLSFNPYKMLEELDKNSRAIKRRLERIKS
Human APOBEC3H (GENBANK ® accession no. NP_861438, such as version
NP_861438.3):
(SEQ ID NO: 120)
MALLTAETFRLQFNNKRRLRRPYYPRKALLCYQLTPQNGSTPTR
GYFENKKKCHAEICFINEIKSMGLDETQCYQVTCYLTWSPCSSCAWELVDFIKAH
DHLNLGIFASRLYYHWCKPQQKGLRLLCGSQVPVEVMGFPEFADCWENFVDHE
KPLSFNPYKMLEELDKNSRAIKRRLERIKQS
Human APOBEC3H (GENBANK ® accession no. NP_001159475, such as
version NP_001159475.2):
(SEQ ID NO: 121)
MALLTAETFRLQFNNKRRLRRPYYPRKALLCYQLTPQ
NGSTPTRGYFENKKKCHAEICFINEIKSMGLDETQCYQVTCYLTWSPCSSCAWEL
VDFIKAHDHLNLGIFASRLYYHWCKPQQKGLRLLCGSQVPVEVMGFPEFADCW
ENFVDHEKPLSFNPYKMLEELDKNSRAIKRRLERIKIPGVRAQGRYMDILCDAEV
Human APOBEC3H (GENBANK ® accession no. ACJ60861, such as version
ACJ60861.1):
(SEQ ID NO: 122)
MALLTAETFRLQFNKRLLRRPYYPRKALLCYQLTPQNGSTPTRGY
FENKKKCHAEICFINEIKSMGLDETQCYQVTCYLTWSPCSSCAWELVDFIKAHDH
LNLRIFASRLYYHWCKPQQDGLRLLCGSQVPVEVMGFPEFADCWENFVDHEKP
LSFNPYKMLEELDKNSRAIKRRLERIKQS
Human APOBEC3H (GENBANK ® accession no. XP_011528294, such as
version XP_011528294.1):
(SEQ ID NO: 123)
MALLTAETFRLQFNNKRRLRRPYYPRKALLCYQLTPQ
NGSTPTRGYFENKKKCHAEICFINEIKSMGLDETQCYQVTCYLTWSPCSSCAWEL
VDFIKAHDHLNLGIFASRLYYHWCKPQQKGLRLLCGSQVPVEVMGFPEFADCW
ENFVDHEKPLSFNPYKMLEELDKNSRAIKRRLERIKIPGVRAQGRYMDILCDAEV
Human APOBEC3H (GENBANK ® accession no. XP_011528293, such as
version XP_011528293.1):
(SEQ ID NO: 124)
MALLTAETFRLQFNNKRRLRRPYYPRKALLCYQLTPQ
NGSTPTRGYFENKKKCHAEICFINEIKSMGLDETQCYQVTCYLTWSPCSSCAWEL
VDFIKAHDHLNLGIFASRLYYHWCKPQQKGLRLLCGSQVPVEVMGFPEFADCW
ENFVDHEKPLSFNPYKMLEELDKNSRAIKRRLERIKGLLLVLLDSRGTCAGSLHG
YIV
Human APOBEC3H (GENBANK ® accession no. XP_011528292, such as
version XP_011528292.1):
(SEQ ID NO: 125)
MALLTAETFRLQFNNKRRLRRPYYPRKALLCYQLTPQ
NGSTPTRGYFENKKKCHAEICFINEIKSMGLDETQCYQVTCYLTWSPCSSCAWEL
VDFIKAHDHLNLGIFASRLYYHWCKPQQKGLRLLCGSQVPVEVMGFPEFADCW
ENFVDHEKPLSFNPYKMLEELDKNSRAIKRRLERIKGLLLVLLDSRGTCAGSLHG
YIV
Human APOBEC3H (GENBANK ® accession no. AGI04217, such as version
AGI04217.1):
(SEQ ID NO: 126)
MALLTAETFRLQFNNKRRLRRPYYPRKALLCYQLTPQNGSTPTRG
YFENKKKCHAEICFINEIKSMGLDETQCYQVTCYLTWSPCSSCAWELVDFIKAHD
HLNLGIFASRLYYHWCKPQQKGLRLLCGSQVPVEVMGFPEFADCWENFVDHEK
PLSFNPYKMLEELDKNSRAIKRRLERIKIPGVRAQGRYMDILCDAEV
Human APOBEC3H (GENBANK ® accession no. AAH69023, such as version
AAH69023.1):
(SEQ ID NO: 127)
MALLTAETFRLQFNNKRRLRRPYYPRKALLCYQLTPQNGSTPTRG
YFENKKKCHAEICFINEIKSMGLDETQCYQVTCYLTWSPCSSCAWELVDFIKAHD
HLNLGIFASRLYYHWCKPQQKGLRLLCGSQVPVEVMGFPEFADCWENFVDHEK
PLSFNPYKMLEELDKNSRAIKRRLERIKS

A representative A3A sequence is set forth in SEQ ID NO: 128 (GENBANK® accession no. NM_145699), which encodes a full length human A3A polypeptide having SEQ ID NO: 129 (UniProt ID P31941). A representative A3B sequence is set forth in SEQ ID NO: 130 (GENBANK® accession no. NM_004900), which encodes a full length human A3B polypeptide having SEQ ID NO: 131 (UniProt ID Q9UH17). Another representative A3B sequence is set forth in SEQ ID NO: 132 (GENBANK® accession no. NP_004891). Another representative A3A sequence is set forth in SEQ ID NO: 133, and includes amino acids 13-199 of human A3A. Another representative A3B sequence is set forth in SEQ ID NO:134, and includes amino acids 193-382 of human A3B. SEQ ID NOS: 133 and 134 contain the C-terminal catalytic domains of A3A and A3B, respectively. SEQ ID NOS: 128-134 are set forth below. Other human and non-human APOBEC sequences are known in the art (e.g., human APOBEC1, AID, APOBEC3C, APOBEC3D, APOBEC3F, and APOBEC3G; GENBANK® accession nos. NP_001635, NP 065712, NP 055323, NP_689639, NP 660341, and NP_068594; SEQ ID NOS:135-140, respectively, as set forth below).

Human APOBEC3A (GENBANK ® accession no. NM_145699, such as version
NM_145699.3):
(SEQ ID NO: 128)
GGAGAAGGGGTGGGGCAGGGTATCGCTGACTCAGCAGCTTCC
AGGTTGCTCTGATGATATATTAAGGCTCCTGAATCCTAAGAGAATGTTGGTGA
AGATCTTAACACCACGCCTTGAGCAAGTCGCAAGAGCGGGAGGACACAGAC
CAGGAACCGAGAAGGGACAAGCACATGGAAGCCAGCCCAGCATCCGGGCCC
AGACACTTGATGGATCCACACATATTCACTTCCAACTTTAACAATGGCATTGG
AAGGCATAAGACCTACCTGTGCTACGAAGTGGAGCGCCTGGACAATGGCACC
TCGGTCAAGATGGACCAGCACAGGGGCTTTCTACACAACCAGGCTAAGAATC
TTCTCTGTGGCTTTTACGGCCGCCATGCGGAGCTGCGCTTCTTGGACCTGGTT
CCTTCTTTGCAGTTGGACCCGGCCCAGATCTACAGGGTCACTTGGTTCATCTC
CTGGAGCCCCTGCTTCTCCTGGGGCTGTGCCGGGGAAGTGCGTGCGTTCCTTC
AGGAGAACACACACGTGAGACTGCGTATCTTCGCTGCCCGCATCTATGATTA
CGACCCCCTATATAAGGAGGCACTGCAAATGCTGCGGGATGCTGGGGCCCAA
GTCTCCATCATGACCTACGATGAATTTAAGCACTGCTGGGACACCTTTGTGGA
CCACCAGGGATGTCCCTTCCAGCCCTGGGATGGACTAGATGAGCACAGCCAA
GCCCTGAGTGGGAGGCTGCGGGCCATTCTCCAGAATCAGGGAAACTGAAGGA
TGGGCCTCAGTCTCTAAGGAAGGCAGAGACCTGGGTTGAGCAGCAGAATAAA
AGATCTTCTTCCAAGAAATGCAAACAGACCGTTCACCACCATCTCCAGCTGCT
CACAGACGCCAGCAAAGCAGTATGCTCCCGATCAAGTAGATTTTTAAAAAAT
CAGAGTGGGCCGGGCGCGGTGGCTCACGCCTGTAATCCCAGCACTTTGGAGG
CCAAGGCGGGTGGATCACGAGGTCAGGAGATCGAGACCATCCTGGCTAACA
CGGTGAAACCCTGTCTCTACTAAAAATACAAAAAATTAGCCAGGCGTGGTGG
CGGGCGCCTGTAGTCCCAGCTACTCTGGAGGCTGAGGCAGGAGAGTAGCGTG
AACCCGGGAGGCAGAGCTTGCGGTGAGCCGAGATTGCGCTACTGCACTCCAG
CCTGGGCGACAGTACCAGACTCCATCTCAAAAAAAAAAAAACCAGACTGAA
TTAATTTTAACTGAAAATTTCTCTTATGTTCCAAGTACACAATAGTAAGATTA
TGCTCAATATTCTCAGAATAATTTTCAATGTATTAATGAAATGAAATGATAAT
TTGGCTTCATATCTAGACTAACACAAAATTAAGAATCTTCCATAATTGCTTTT
GCTCAGTAACTGTGTCATGAATTGCAAGAGTTTCCACAAACACT
Human APOBEC3A (UniProt ID P31941, such as version P31941.3):
(SEQ ID NO: 129)
MEASPASGPRHLMDPHIFTSNFNNGIGRHKTYLCYEVERLDNGTSVKMDQHRGFLHNQAK
NLLCGFYGRHAELRFLDLVPSLQLDPAQIYRVTWFISWSPCFSWGCAGEVRAFL
QENTHVRLRIFAARIYDYDPLYKEALQMLRDAGAQVSIMTYDEFKHCWDTFVD
HQGCPFQPWDGLDEHSQALSGRLRAILQNQGN
Human APOBEC3B (GENBANK ® accession no. NM_004900, such as version
NM_004900.4):
(SEQ ID NO: 130)
CACAGAGCTTCAAAAAAAGAGCGGGACAGGGACAAGCGTAT
CTAAGAGGCTGAACATGAATCCACAGATCAGAAATCCGATGGAGCGGATGTA
TCGAGACACATTCTACGACAACTTTGAAAACGAACCCATCCTCTATGGTCGG
AGCTACACTTGGCTGTGCTATGAAGTGAAAATAAAGAGGGGCCGCTCAAATC
TCCTTTGGGACACAGGGGTCTTTCGAGGCCAGGTGTATTTCAAGCCTCAGTAC
CACGCAGAAATGTGCTTCCTCTCTTGGTTCTGTGGCAACCAGCTGCCTGCTTA
CAAGTGTTTCCAGATCACCTGGTTTGTATCCTGGACCCCCTGCCCGGACTGTG
TGGCGAAGCTGGCCGAATTCCTGTCTGAGCACCCCAATGTCACCCTGACCAT
CTCTGCCGCCCGCCTCTACTACTACTGGGAAAGAGATTACCGAAGGGCGCTC
TGCAGGCTGAGTCAGGCAGGAGCCCGCGTGAAGATCATGGACTATGAAGAAT
TTGCATACTGCTGGGAAAACTTTGTGTACAATGAAGGTCAGCAATTCATGCCT
TGGTACAAATTCGATGAAAATTATGCATTCCTGCACCGCACGCTAAAGGAGA
TTCTCAGATACCTGATGGATCCAGACACATTCACTTTCAACTTTAATAATGAC
CCTTTGGTCCTTCGACGGCGCCAGACCTACTTGTGCTATGAGGTGGAGCGCCT
GGACAATGGCACCTGGGTCCTGATGGACCAGCACATGGGCTTTCTATGCAAC
GAGGCTAAGAATCTTCTCTGTGGCTTTTACGGCCGCCATGCGGAGCTGCGCTT
CTTGGACCTGGTTCCTTCTTTGCAGTTGGACCCGGCCCAGATCTACAGGGTCA
CTTGGTTCATCTCCTGGAGCCCCTGCTTCTCCTGGGGCTGTGCCGGGGAAGTG
CGTGCGTTCCTTCAGGAGAACACACACGTGAGACTGCGCATCTTCGCTGCCC
GCATCTATGATTACGACCCCCTATATAAGGAGGCGCTGCAAATGCTGCGGGA
TGCTGGGGCCCAAGTCTCCATCATGACCTACGATGAGTTTGAGTACTGCTGGG
ACACCTTTGTGTACCGCCAGGGATGTCCCTTCCAGCCCTGGGATGGACTAGA
GGAGCACAGCCAAGCCCTGAGTGGGAGGCTGCGGGCCATTCTCCAGAATCAG
GGAAACTGAAGGATGGGCCTCAGTCTCTAAGGAAGGCAGAGACCTGGGTTG
AGCAGCAGAATAAAAGATCTTCTTCCAAGAAATGCAAACAGACCGTTCACCA
CCATCTCCAGCTGCTCACAGACACCAGCAAAGCAATGTGCTCCTGATCAAGT
AGATTTTTTAAAAATCAGAGTCAATTAATTTTAATTGAAAATTTCTCTTATGTT
CCAAGTGTACAAGAGTAAGATTATGCTCAATATTCCCAGAATAGTTTTCAATG
TATTAATGAAGTGATTAATTGGCTCCATATTTAGACTAATAAAACATTAAGAA
TCTTCCATAATTGTTTCCACAAACACTAAAAAAAAAAAAAAAAAAAAAAA
Human APOBEC3B major allele (UniProt ID Q9UH17, such as version
Q9UH17.1):
(SEQ ID NO: 131)
MNPQIRNPMERMYRDTFYDNFENEPILYGRSYTWLCYEVKIKRGRS
NLLWDTGVFRGQVYFKPQYHAEMCFLSWFCGNQLPAYKCFQITWFVSWTPCPD
CVAKLAEFLSEHPNVTLTISAARLYYYWERDYRRALCRLSQAGARVTIMDYEEF
AYCWENFVYNEGQQFMPWYKFDENYAFLHRTLKEILRYLMDPDTFTFNFNNDP
LVLRRRQTYLCYEVERLDNGTWVLMDQHMGFLCNEAKNLLCGFYGRHAELRF
LDLVPSLQLDPAQIYRVTWFISWSPCFSWGCAGEVRAFLQENTHVRLRIFAARIY
DYDPLYKEALQMLRDAGAQVSIMTYDEFEYCWDTFVYRQGCPFQPWDGLEEHS
QALSGRLRAILQNQGN
Human APOBEC3B minor allele (GENBANK ® accession no. NP_004891, such
as version NP_004891.4):
(SEQ ID NO: 132)
MNPQIRNPMERMYRDTFYDNFENEPILYGRSYTWLCY
EVKIKRGRSNLLWDTGVFRGQVYFKPQYHAEMCFLSWFCGNQLPAYKCFQITW
FVSWTPCPDCVAKLAEFLSEHPNVTLTISAARLYYYWERDYRRALCRLSQAGAR
VKIMDYEEFAYCWENFVYNEGQQFMPWYKFDENYAFLHRTLKEILRYLMDPDT
FTFNFNNDPLVLRRRQTYLCYEVERLDNGTWVLMDQHMGFLCNEAKNLLCGFY
GRHAELRFLDLVPSLQLDPAQIYRVTWFISWSPCFSWGCAGEVRAFLQENTHVR
LRIFAARIYDYDPLYKEALQMLRDAGAQVSIMTYDEFEYCWDTFVYRQGCPFQP
WDGLEEHSQALSGRLRAILQNQGN
Human APOBEC3 A amino acids 13-199:
(SEQ ID NO: 133)
MDPHIFTSNFNNGIGRHKTYLCYE
VERLDNGTSVKMDQHRGFLHNQAKNLLCGFYGRHAELRFLDLVPSLQLDPAQI
YRVTWFISWSPCFSWGCAGEVRAFLQENTHVRLRIFAARIYDYDPLYKEALQML
RDAGAQVSIIVITYDEFKHCWDTFVDHQGCPFQPWDGLDEHSQALSGRLRAILQN
QGN
Human APOBEC3B amino acids 193-382:
(SEQ ID NO: 134)
MDPDTFTFNFNNDPLVLRRRQTY
LCYEVERLDNGTWVLMDQHMGFLCNEAKNLLCGFYGRHAELRFLDLVPSLQLD
PAQIYRVTWFISWSPCFSWGCAGEVRAFLQENTHVRLRIFAARIYDYDPLYKEAL
QMLRDAGAQVSIIVITYDEFEYCWDTFVYRQGCPFQPWDGLEEHSQALSGRLRAI
LQNQGN
Human APOBEC1 (GENBANK ® accession no. NP_001635, such as version
NP_001635.2):
(SEQ ID NO: 135)
MTSEKGPSTGDPTLRRRIEPWEFDVFYDPRELRKEACLLYEIKWG
MSRKIWRSSGKNTTNHVEVNFIKKFTSERDFHPSMSCSITWFLSWSPCWECSQAI
REFLSRHPGVTLVIYVARLFWHMDQQNRQGLRDLVNSGVTIQIIVIRASEYYHCW
RNFVNYPPGDEAHWPQYPPLWMMLYALELHCIILSLPPCLKISRRWQNHLTFFRL
HLQNCHYQTIPPHILLATGLIHPSVAWR
Human AID (GENBANK ® accession no. NP_065712, such as version
NP_065712.3):
(SEQ ID NO: 136)
MDSLLMNRRKFLYQFKNVRWAKGRRETYLCYVVKRRDSATSF
SLDFGYLRNKNGCHVELLFLRYISDWDLDPGRCYRVTWFTSWSPCYDCARHVA
DFLRGNPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL
Human APOBEC3C (GENBANK ® accession no. NP_055323, such as version
NP_055323.2):
(SEQ ID NO: 137)
MNPQIRNPMKAMYPGTFYFQFKNLWEANDRNETWLCFTVEGIK
RRSVVSWKTGVFRNQVDSETHCHAERCFLSWFCDDILSPNTKYQVTWYTSWSP
CPDCAGEVAEFLARHSNVNLTIFTARLYYFQYPCYQEGLRSLSQEGVAVEIMDY
EDFKYCWENFVYNDNEPFKPWKGLKTNFRLLKRRLRESLQ
Human APOBEC3D (GENBANK ® accession no. NP_689639, such as version
NP_689639.2):
(SEQ ID NO: 138)
MNPQIRNPMERMYRDTFYDNFENEPILYGRSYTWLCYEVKIKRG
RSNLLWDTGVFRGPVLPKRQSNHRQEVYFRFENHAEMCFLSWFCGNRLPANRR
FQITWFVSWNPCLPCVVKVTKFLAEHPNVTLTISAARLYYYRDRDWRWVLLRL
HKAGARVKIMDYEDFAYCWENFVCNEGQPFMPWYKFDDNYASLHRTLKEILRN
PMEAMYPHIFYFHFKNLLKACGRNESWLCFTMEVTKHHSAVFRKRGVFRNQVD
PETHCHAERCFLSWFCDDILSPNTNYEVTWYTSWSPCPECAGEVAEFLARHSNV
NLTIFTARLCYFWDTDYQEGLCSLSQEGASVKEVIGYKDFVSCWKNFVYSDDEPF
KPWKGLQTNFRLLKRRLREILQ
Human APOBEC3F (GENBANK ® accession no. NP_660341, such as version
NP_660341.2):
(SEQ ID NO: 139)
MKPHFRNTVERMYRDTFSYNFYNRPILSRRNTVWLCYEVKTKGP
SRPRLDAKIFRGQVYSQPEHHAEMCFLSWFCGNQLPAYKCFQITWFVSWTPCPD
CVAKLAEFLAEHPNVTLTISAARLYYYWERDYRRALCRLSQAGARVKIMDDEEF
AYCWENFVYSEGQPFMPWYKFDDNYAFLHRTLKEILRNPMEAMYPHIFYFHFK
NLRKAYGRNESWLCFTMEVVKHEISPVSWKRGVFRNQVDPETHCHAERCFLSW
FCDDILSPNTNYEVTWYTSWSPCPECAGEVAEFLARHSNVNLTIFTARLYYFWDT
DYQEGLRSLSQEGASVEIMGYKDFKYCWENFVYNDDEPFKPWKGLKYNFLFLD
SKLQEILE
Human APOBEC3G (GENBANK ® accession no. NP_068594, such as version
NP_068594.1):
(SEQ ID NO: 140)
MKPHFRNTVERMYRDTFSYNFYNRPILSRRNTVWLCYEVKTKGP
SRPPLDAKIFRGQVYSELKYHPEMRFFHWFSKWRKLHRDQEYEVTWYISWSPCT
KCTRDMATFLAEDPKVTLTIFVARLYYFWDPDYQEALRSLCQKRDGPRATMKI
MNYDEFQHCWSKFVYSQRELFEPWNNLPKYYILLHIMLGEILRHSMDPPTFTFNF
NNEPWVRGRHETYLCYEVERMHNDTWVLLNQRRGFLCNQAPHKHGFLEGRHA
ELCFLDVIPFWKLDLDQDYRVTCFTSWSPCFSCAQEMAKFISKNKHVSLCIFTARI
YDDQGRCQEGLRTLAEAGAKISIMTYSEFKHCWDTFVDHQGCPFQPWDGLDEH
SQDLSGRLRAILQNQEN

A variant APOBEC polypeptide as provided herein can include a full-length amino acid sequence of an APOBEC protein, or a catalytic fragment of an APOBEC protein (e.g., a fragment that includes the C-terminal catalytic domain). In some embodiments, a variant APOBEC polypeptide as provided herein can have an amino acid sequence that is at least about 90% identical to a reference APOBEC amino acid sequence or a fragment thereof. For example, an A3H polypeptide as provided herein can have an amino acid sequence that is at least 90% identical (e.g., at least about 91%, 92%, 93%, 94%, 95%, 96%, 97%, 98%, 99%, 99.5%, or 99.8% identical) to a reference A3H amino acid sequence (e.g., SEQ ID NO:81 or SEQ ID NO:83, or a fragment thereof), and an A3H nucleic acid as provided herein can have a nucleotide sequence that is at least 90% identical (e.g., at least about 91%, 92%, 93%, 94%, 95%, 96%, 97%, 98%, 99%, 99.5%, or 99.8% identical) to a reference A3H nucleotide sequence. The APOBEC-Cas9 fusion polypeptides provided herein also can contain such variant APOBEC polypeptide sequences.

The amino acid sequence of a variant APOBEC polypeptide can have one, two, three, four, five, six, seven, eight, night, ten, or more than ten amino acid substitutions, deletions, or additions as compared to a reference APOBEC amino acid sequence. In some embodiments, the variant polypeptide can include one or more amino acid substitutions, deletions, or additions within, for example, the loop 7 region and/or a positive patch within the APOBEC polypeptide. For example, a variant APOBEC polypeptide can have a substitution at one or more hydrophobic residues within the loop 7 region. In some cases, a variant APOBEC polypeptide can have a substitution at one or more basic residues with a positive patch.

In some embodiments, for example, a variant A3H polypeptide can have a substitution at one or more positions corresponding to positions 18, 20, 114, 115, 172, 175, 176, and 179 of the reference A3H sequence set forth in SEQ ID NO:81. In some cases, for example, a variant A3H polypeptide can have a glutamic acid substituted for the arginine at position 18 of SEQ ID NO:81, a glutamic acid substituted for the arginine at position 20 of SEQ ID NO:81, an alanine substituted for the histidine at position 114 of SEQ ID NO:81, an alanine substituted for the tryptophan at position 115 of SEQ ID NO:81, a glutamic acid substituted for the alanine at position 172 of SEQ ID NO:81, a glutamic acid substituted for the arginine at position 175 of SEQ ID NO:81, a glutamic acid substituted for the arginine at position 176 of SEQ ID NO:81, a glutamic acid substituted for the arginine at position 179 of SEQ ID NO:81, or any combination thereof. Other APOBEC polypeptides can have a substitution at one or more positions within a positive patch, for example, or at one or more residues homologous to the AH3 RNA binding residues disclosed herein.

In some cases, a variant APOBEC polypeptide (e.g., a variant A3B N-terminal domain (ntd) polypeptide) can include a substitution at one or more basic positions (Xiao et al., Nucl Acids Res 45(12):7494-7506, 2017), such as one or more of positions 40, 42, 43, 45, 62, 84, 103, 132, 133, and 144 as compared to the reference A3B sequences set forth in SEQ ID NOS:131 and 132.

In some cases, a variant APOBEC polypeptide (e.g., a variant A3G polypeptide) can include a substitution at one or more basic or hydrophobic positions (Xiao et al., Nat Commun 7:12193, doi: 10.1038/ncomms12193, 2016), such as one or more of positions 24, 94, 124, 126, 127, 180, and 184 as compared to the reference A3G sequence set forth in SEQ ID NO:140.

The amino acid positions within the A3A and A3B enzymes that determine the −1 nucleobase preference have been identified, as discussed in U.S. Publication No. 2018/0170984. In particular, evidence disclosed therein indicates that the aspartic acid residue at position 131 of human A3A (UniProt ID P31941) or position 314 of human A3B (UniProt ID Q9UH17) is a determinant of APOBEC target specificity. Further evidence of this was provided by replacing the aspartic acid residue at position 131 of A3A with glutamic acid, and replacing the aspartic acid at position 314 of A3B with glutamic acid, which resulted altered local target site specificity from TC to CC (Shi et al., Nature Struct Mol Biol 24:131-139, 2017). This position is within loop 7 of A3A and loop 7 of the A3B catalytic domain (ctd) proteins. Variant APOBEC polypeptides that have altered preference for the nucleobase at the −1 position therefore are provided herein, together with nucleic acids encoding the variant APOBEC polypeptides, host cells containing the nucleic acids, and methods for using the polypeptides and nucleic acids for targeted DNA modification. These variant APOBEC polypeptides can include mutations within loop 7, for example. In some cases, a variant APOBEC polypeptide be a loop-exchanged chimera, with amino acids from loop 7 of a different APOBEC polypeptide substituted for the loop 7 amino acids normally found within the polypeptide. For example, in some embodiments, amino acids present within loop 7 of A3A can be replaced by amino acids from the corresponding portion of A3G or AID. By coupling a variant APOBEC polypeptide sequence to a targeting molecule, it is possible to modify selected DNA sequences in a highly specific manner. Thus, fusion polypeptides containing a variant APOBEC portion and a targeting (e.g., Cas9) portion also are provided herein, as are nucleic acids encoding the variant A3H and A3H-Cas9 fusion polypeptides, host cells containing the nucleic acids, and methods for using the polypeptides and nucleic acids for targeted DNA modification.

The CRISPR/Cas system includes components of a prokaryotic adaptive immune system that is functionally analogous to eukaryotic RNA interference, using RNA base pairing to direct DNA or RNA cleavage. The Cas9 protein functions as an endonuclease, and CRISPR RNA (crRNA) and trans-activating RNA (tracrRNA) sequences complex with the Cas9 enzyme and direct it to a target DNA sequence (Makarova et al., Nat Rev Microbiol 9(6):467-477, 2011). The modification of a single targeting RNA can be sufficient to alter the nucleotide target of a Cas protein. In some cases, crRNA and tracrRNA can be engineered as a single cr/tracrRNA hybrid (also referred to as a “guide RNA” or “gRNA”) to direct Cas9 cleavage activity (Jinek et al., Science, 337(6096):816-821, 2012). The CRISPR/Cas system can be used in a variety of prokaryotic and eukaryotic organisms (see, e.g., Jiang et al., Nat Biotechnol, 31(3):233-239, 2013; Dicarlo et al., Nucleic Acids Res, doi:10.1093/nar/gkt135, 2013; Cong et al., Science, 339(6121):819-823, 2013; Mali et al., Science, 339(6121):823-826, 2013; Cho et al., Nat Biotechnol, 31(3):230-232, 2013; and Hwang et al., Nat Biotechnol, 31(3):227-229, 2013).

CRISPR clusters are transcribed and processed into crRNA; the correct processing into crRNA requires a trans-encoded small tracrRNA. The combination of Cas9, crRNA, and tracrRNA can then cleave linear or circular dsDNA targets that are complementary to a spacer within the CRISPR cluster. Cas9 recognizes a short protospacer adjacent motif (PAM) in the CRISPR repeat sequences, which aids in distinguishing self from non-self. Cas9 nuclease sequences and structures are well known to those of skill in the art (see, e.g., Ferretti et al., Proc Natl Acad Sci USA 98:4658-4663, 2001; Deltcheva et al., Nature 471:602-607, 2011; and Jinek Science 337:816-821, 2012). Cas9 orthologs also have been described in species such as S. pyogenes and S. thermophilus.

The homology region within the crRNA sequence (the sequence that targets the crRNA to the desired DNA sequence) can be between about 10 and about 40 (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40) nucleotides in length. The tracrRNA hybridizing region within each crRNA sequence can be between about 8 and about 20 (e.g., 8, 9, 10, 11, 12, 13, 14, 15, 16, 17, 18, 19, or 20) nucleotides in length. The overall length of a crRNA sequence can be, for example, between about 20 and about 80 (e.g., 20, 25, 30, 35, 40, 45, 50, 55, 60, 65, 70, 75, or 80) nucleotides, while the overall length of a tracrRNA can be, for example, between about 10 and about 30 (e.g., 10, 12, 14, 16, 18, 20, 22, 24, 26, 28, or 30) nucleotides. The overall length of a gRNA sequence, which includes a homology region and a stem loop region that contains a crRNA/tracrRNA hybridizing region and a linker-loop sequence, can be between about 30 and about 110 (e.g., 30, 35, 40, 45, 50, 55, 60, 65, 70, 75, 80, 85, 90, 95, 100, 105, 110, 115, 120, 125, or 130) nucleotides.

In some embodiments, the Cas9 portion of the fusion polypeptides provided herein can include the non-catalytic portion of a wild-type Cas9 polypeptide, or a Cas9 polypeptide containing one or more mutations (e.g., substitutions, deletions, or additions) within its amino acid sequence as compared to the amino acid sequence of a corresponding wild-type Cas9 protein, where the mutant Cas9 does not have nuclease activity. In some embodiments, additional amino acids may be added to the N- and/or C-terminus. For example, Cas9 protein can be modified by the addition of a VP64 activation domain or a green fluorescent protein to the C-terminus, or by the addition of nuclear-localization signals to both the N- and C-termini (see, e.g., Mali et al. Nature Biotechnol 31:833-838, 2013; and Cong et al. Science 339:819-823). A representative Cas9 nucleic acid sequence is set forth in SEQ ID NO:85, and a representative Cas9 amino acid sequence is set forth in SEQ ID NO:86.

Streptococcus pyogenes (NCBI Ref NC_017053.1) Cas9:
(SEQ ID NO: 85)
ATGGATAAGAAA
TACTCAATAGGCTTAGATATCGGCACAAATAGCGTCGGATGGGCGGTGATCA
CTGATGATTATAAGGTTCCGTCTAAAAAGTTCAAGGTTCTGGGAAATACAGA
CCGCCACAGTATCAAAAAAAATCTTATAGGGGCTCTTTTATTTGGCAGTGGA
GAGACAGCGGAAGCGACTCGTCTCAAACGGACAGCTCGTAGAAGGTATACA
CGTCGGAAGAATCGTATTTGTTATCTACAGGAGATTTTTTCAAATGAGATGGC
GAAAGTAGATGATAGTTTCTTTCATCGACTTGAAGAGTCTTTTTTGGTGGAAG
AAGACAAGAAGCATGAACGTCATCCTATTTTTGGAAATATAGTAGATGAAGT
TGCTTATCATGAGAAATATCCAACTATCTATCATCTGCGAAAAAAATTGGCA
GATTCTACTGATAAAGCGGATTTGCGCTTAATCTATTTGGCCTTAGCGCATAT
GATTAAGTTTCGTGGTCATTTTTTGATTGAGGGAGATTTAAATCCTGATAATA
GTGATGTGGACAAACTATTTATCCAGTTGGTACAAATCTACAATCAATTATTT
GAAGAAAACCCTATTAACGCAAGTAGAGTAGATGCTAAAGCGATTCTTTCTG
CACGATTGAGTAAATCAAGACGATTAGAAAATCTCATTGCTCAGCTCCCCGG
TGAGAAGAGAAATGGCTTGTTTGGGAATCTCATTGCTTTGTCATTGGGATTGA
CCCCTAATTTTAAATCAAATTTTGATTTGGCAGAAGATGCTAAATTACAGCTT
TCAAAAGATACTTACGATGATGATTTAGATAATTTATTGGCGCAAATTGGAG
ATCAATATGCTGATTTGTTTTTGGCAGCTAAGAATTTATCAGATGCTATTTTA
CTTTCAGATATCCTAAGAGTAAATAGTGAAATAACTAAGGCTCCCCTATCAG
CTTCAATGATTAAGCGCTACGATGAACATCATCAAGACTTGACTCTTTTAAAA
GCTTTAGTTCGACAACAACTTCCAGAAAAGTATAAAGAAATCTTTTTTGATCA
ATCAAAAAACGGATATGCAGGTTATATTGATGGGGGAGCTAGCCAAGAAGA
ATTTTATAAATTTATCAAACCAATTTTAGAAAAAATGGATGGTACTGAGGAA
TTATTGGTGAAACTAAATCGTGAAGATTTGCTGCGCAAGCAACGGACCTTTG
ACAACGGCTCTATTCCCCATCAAATTCACTTGGGTGAGCTGCATGCTATTTTG
AGAAGACAAGAAGACTTTTATCCATTTTTAAAAGACAATCGTGAGAAGATTG
AAAAAATCTTGACTTTTCGAATTCCTTATTATGTTGGTCCATTGGCGCGTGGC
AATAGTCGTTTTGCATGGATGACTCGGAAGTCTGAAGAAACAATTACCCCAT
GGAATTTTGAAGAAGTTGTCGATAAAGGTGCTTCAGCTCAATCATTTATTGAA
CGCATGACAAACTTTGATAAAAATCTTCCAAATGAAAAAGTACTACCAAAAC
ATAGTTTGCTTTATGAGTATTTTACGGTTTATAACGAATTGACAAAGGTCAAA
TATGTTACTGAGGGAATGCGAAAACCAGCATTTCTTTCAGGTGAACAGAAGA
AAGCCATTGTTGATTTACTCTTCAAAACAAATCGAAAAGTAACCGTTAAGCA
ATTAAAAGAAGATTATTTCAAAAAAATAGAATGTTTTGATAGTGTTGAAATTT
CAGGAGTTGAAGATAGATTTAATGCTTCATTAGGCGCCTACCATGATTTGCTA
AAAATTATTAAAGATAAAGATTTTTTGGATAATGAAGAAAATGAAGATATCT
TAGAGGATATTGTTTTAACATTGACCTTATTTGAAGATAGGGGGATGATTGAG
GAAAGACTTAAAACATATGCTCACCTCTTTGATGATAAGGTGATGAAACAGC
TTAAACGTCGCCGTTATACTGGTTGGGGACGTTTGTCTCGAAAATTGATTAAT
GGTATTAGGGATAAGCAATCTGGCAAAACAATATTAGATTTTTTGAAATCAG
ATGGTTTTGCCAATCGCAATTTTATGCAGCTGATCCATGATGATAGTTTGACA
TTTAAAGAAGATATTCAAAAAGCACAGGTGTCTGGACAAGGCCATAGTTTAC
ATGAACAGATTGCTAACTTAGCTGGCAGTCCTGCTATTAAAAAAGGTATTTTA
CAGACTGTAAAAATTGTTGATGAACTGGTCAAAGTAATGGGGCATAAGCCAG
AAAATATCGTTATTGAAATGGCACGTGAAAATCAGACAACTCAAAAGGGCCA
GAAAAATTCGCGAGAGCGTATGAAACGAATCGAAGAAGGTATCAAAGAATT
AGGAAGTCAGATTCTTAAAGAGCATCCTGTTGAAAATACTCAATTGCAAAAT
GAAAAGCTCTATCTCTATTATCTACAAAATGGAAGAGACATGTATGTGGACC
AAGAATTAGATATTAATCGTTTAAGTGATTATGATGTCGATCACATTGTTCCA
CAAAGTTTCATTAAAGACGATTCAATAGACAATAAGGTACTAACGCGTTCTG
ATAAAAATCGTGGTAAATCGGATAACGTTCCAAGTGAAGAAGTAGTCAAAAA
GATGAAAAACTATTGGAGACAACTTCTAAACGCCAAGTTAATCACTCAACGT
AAGTTTGATAATTTAACGAAAGCTGAACGTGGAGGTTTGAGTGAACTTGATA
AAGCTGGTTTTATCAAACGCCAATTGGTTGAAACTCGCCAAATCACTAAGCA
TGTGGCACAAATTTTGGATAGTCGCATGAATACTAAATACGATGAAAATGAT
AAACTTATTCGAGAGGTTAAAGTGATTACCTTAAAATCTAAATTAGTTTCTGA
CTTCCGAAAAGATTTCCAATTCTATAAAGTACGTGAGATTAACAATTACCATC
ATGCCCATGATGCGTATCTAAATGCCGTCGTTGGAACTGCTTTGATTAAGAAA
TATCCAAAACTTGAATCGGAGTTTGTCTATGGTGATTATAAAGTTTATGATGT
TCGTAAAATGATTGCTAAGTCTGAGCAAGAAATAGGCAAAGCAACCGCAAA
ATATTTCTTTTACTCTAATATCATGAACTTCTTCAAAACAGAAATTACACTTG
CAAATGGAGAGATTCGCAAACGCCCTCTAATCGAAACTAATGGGGAAACTGG
AGAAATTGTCTGGGATAAAGGGCGAGATTTTGCCACAGTGCGCAAAGTATTG
TCCATGCCCCAAGTCAATATTGTCAAGAAAACAGAAGTACAGACAGGCGGAT
TCTCCAAGGAGTCAATTTTACCAAAAAGAAATTCGGACAAGCTTATTGCTCGT
AAAAAAGACTGGGATCCAAAAAAATATGGTGGTTTTGATAGTCCAACGGTAG
CTTATTCAGTCCTAGTGGTTGCTAAGGTGGAAAAAGGGAAATCGAAGAAGTT
AAAATCCGTTAAAGAGTTACTAGGGATCACAATTATGGAAAGAAGTTCCTTT
GAAAAAAATCCGATTGACTTTTTAGAAGCTAAAGGATATAAGGAAGTTAAAA
AAGACTTAATCATTAAACTACCTAAATATAGTCTTTTTGAGTTAGAAAACGGT
CGTAAACGGATGCTGGCTAGTGCCGGAGAATTACAAAAAGGAAATGAGCTG
GCTCTGCCAAGCAAATATGTGAATTTTTTATATTTAGCTAGTCATTATGAAAA
GTTGAAGGGTAGTCCAGAAGATAACGAACAAAAACAATTGTTTGTGGAGCAG
CATAAGCATTATTTAGATGAGATTATTGAGCAAATCAGTGAATTTTCTAAGCG
TGTTATTTTAGCAGATGCCAATTTAGATAAAGTTCTTAGTGCATATAACAAAC
ATAGAGACAAACCAATACGTGAACAAGCAGAAAATATTATTCATTTATTTAC
GTTGACGAATCTTGGAGCTCCCGCTGCTTTTAAATATTTTGATACAACAATTG
ATCGTAAACGATATACGTCTACAAAAGAAGTTTTAGATGCCACTCTTATCCAT
CAATCCATCACTGGTCTTTATGAAACACGCATTGATTTGAGTCAGCTAGGAGG
TGACTGA
S. pyogenes Cas9 protein:
(SEQ ID NO: 86)
MDKKYSIGLDIGTNSVGWAVITDDYKVPSKKF
KVLGNTDRHSIKKNLIGALLFGSGETAEATRLKRTARRRYTRRKNRICYLQEIFSN
EMAKVDDSFFHRLEESFLVEEDKKHERHPIFGNIVDEVAYHEKYPTIYHLRKKLA
DSTDKADLRLIYLALAHMIKFRGHFLIEGDLNPDNSDVDKLFIQLVQIYNQLFEEN
PINASRVDAKAILSARLSKSRRLENLIAQLPGEKRNGLFGNLIALSLGLTPNFKSNF
DLAEDAKLQLSKDTYDDDLDNLLAQIGDQYADLFLAAKNLSDAILLSDILRVNSE
ITKAPLSASMIKRYDEHHQDLTLLKALVRQQLPEKYKEIFFDQSKNGYAGYIDGG
ASQEEFYKFIKPILEKMDGTEELLVKLNREDLLRKQRTEDNGSIPHQIHLGELHAI
LRRQEDFYPFLKDNREKIEKILTFRIPYYVGPLARGNSRFAWMTRKSEETITPWNF
EEVVDKGASAQSFIERMTNFDKNLPNEKVLPKHSLLYEYFTVYNELTKVKYVTE
GMRKPAFLSGEQKKAIVDLLEKTNRKVTVKQLKEDYFKKIECEDSVEISGVEDRF
NASLGAYHDLLKIIKDKDELDNEENEDILEDIVLTLTLFEDRGMIEERLKTYAHLF
DDKVMKQLKRRRYTGWGRLSRKLINGIRDKQSGKTILDFLKSDGFANRNFMQLI
HDDSLTFKEDIQKAQVSGQGHSLHEQIANLAGSPAIKKGILQTVKIVDELVKVMG
HKPENIVIEMARENQTTQKGQKNSRERMKRIEEGIKELGSQILKEHPVENTQLQN
EKLYLYYLQNGRDMYVDQELDINRLSDYDVDHIVPQSFIKDDSIDNKVLTRSDK
NRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIA
KSEQEIGKATAKYFFYSNIMNFEKTEITLANGEIRKRPLIETNGETGEIVWDKGRD
FATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYE
KLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGL
YETRIDLSQLGGD.

An APOBEC-Cas9 fusion polypeptide as provided herein can include the full-length amino acid sequence of a Cas9 protein, or a fragment of a Cas9 protein. Typically, the APOBEC-Cas9 fusion polypeptides provided herein include a Cas9 fragment that can bind to a crRNA and tracrRNA (or a gRNA), but does not include a functional nuclease domain. For example, the fusion may contain a non-functional nuclease domain, or a portion of a nuclease domain that is not sufficient to confer nuclease activity, or may lack a nuclease domain altogether. Thus, in some cases, an A3H-Cas9 fusion polypeptide can contain a fragment of Cas9, such as a fragment including the Cas9 gRNA binding domain, or a fragment that includes both the gRNA binding domain and an inactivated version of the DNA cleavage domain. The Cas portion of an A3H-Cas9 fusion also may contain a variant Cas polypeptide having an amino acid sequence that is at least about 90% identical to a wild-type Cas9 sequence (e.g., at least about 91%, 92%, 93%, 94%, 95%, 96%, 97%, 98%, 99%, 99.5%, or 99.8% identical to a wild-type Cas9 amino acid sequence).

In some embodiments, the fusion polypeptides provided herein can include a “nuclease-dead” Cas9 polypeptide that lacks nuclease activity and may or may not have nickase activity (such that it cuts one strand of a double-stranded DNA), but can bind to a preselected target sequence when complexed with crRNA and tracrRNA (or gRNA). Without being bound by a particular mechanism, the use of a DNA targeting polypeptide with nickase activity, where the nickase generates a strand-specific cut on the strand opposing the uracil to be modified, can have the subsequent effect of directing repair machinery to non-modified strand, resulting in repair of the nick so both strands are modified. For example, with respect to the Cas9 sequence of SEQ ID NO:86, a Cas9 polypeptide can be a D100A Cas9 polypeptide (or a portion thereof) that has nickase activity but not nuclease activity, or a H840A Cas9 polypeptide (or a portion thereof) that has nickase activity but not nuclease activity.

In some embodiments, a “nuclease-dead” polypeptide can be a D10A H840A Cas9 polypeptide (or a portion thereof) that has neither nickase nor nuclease activity. A Cas9 polypeptide also can be a D10A D839A H840A N863A Cas9 polypeptide in which alanine residues are substituted for the aspartic acid residues at positions 10 and 839, the histidine residue at position 840, and the asparagine residue at position 863 (with respect to SEQ ID NO:86). See, e.g., Mali et al., Nature Biotechnol, supra; Jinek et al., supra; and Qi et al., Cell 152(5):1173-83, 2013.

An exemplary reference Cas9 amino acid sequence having an inactivated nuclease domain with D10A and H840A mutations (underlined) is: MDKKYSIGLAIGTNSVGW AVITDEYKVPSKKFKVLGNTDRHSIKKNLIGALLFDSGETAEATRLKRTARRRYT RRKNRICYLQEIFSNEMAKVDDSFFHRLEESFLVEEDKKHERHPIFGNIVDEVAY HEKYPTIYHLRKKLVDSTDKADLRLIYLALAHMIKFRGHFLIEGDLNPDNSDVDK LFIQLVQTYNQLFEENPINASGVDAKAILSARLSKSRRLENLIAQLPGEKKNGLFG NLIALSLGLTPNFKSNFDLAEDAKLQLSKDTYDDDLDNLLAQIGDQYADLFLAA KNLSDAILLSDILRVNTEITKAPLSASMIKRYDEHHQDLTLLKALVRQQLPEKYKE IFFDQSKNGYAGYIDGGASQEEFYKFIKPILEKMDGTEELLVKLNREDLLRKQRTF DNGSIPHQIHLGELHAILRRQEDFYPFLKDNREKIEKILTFRIPYYVGPLARGNSRF AWMTRKSEETITPWNFEEVVDKGASAQSFIERMTNFDKNLPNEKVLPKHSLLYE YFTVYNELTKVKYVTEGMRKPAFLSGEQKKAIVDLLFKTNRKVTVKQLKEDYF KKIECFDSVEISGVEDRFNASLGTYHDLLKIIKDKDFLDNEENEDILEDIVLTLTLF EDREMIEERLKTYAHLFDDKVMKQLKRRRYTGWGRLSRKLINGIRDKQSGKTIL DFLKSDGFANRNFMQLIHDDSLTFKEDIQKAQVSGQGDSLHEHIANLAGSPAIKK GILQTVKVVDELVKVMGRHKPENIVIEMARENQTTQKGQKNSRERMKRIEEGIK ELGSQILKEHPVENTQLQNEKLYLYYLQNGRDMYVDQELDINRLSDYDVDAIVP QSFLKDDSIDNKVLTRSDKNRGKSDNVPSEEVVKKMKNYWRQLLNAKLITQRK FDNLTKAERGGLSELDKAGFIKRQLVETRQITKHVAQILDSRMNTKYDENDKLIR EVKVITLKSKLVSDFRKDFQFYKVREINNYHHAHDAYLNAVVGTALIKKYPKLE SEFVYGDYKVYDVRKMIAKSEQEIGKATAKYFFYSNIMNFFKTEITLANGEIRKR PLIETNGETGEIVWDKGRDFATVRKVLSMPQVNIVKKTEVQTGGFSKESILPKRN SDKLIARKKDWDPKKYGGFDSPTVAYSVLVVAKVEKGKSKKLKSVKELLGITIM ERSSFEKNPIDFLEAKGYKEVKKDLIIKLPKYSLFELENGRKRMLASAGELQKGN ELALPSKYVNFLYLASHYEKLKGSPEDNEQKQLFVEQHKHYLDEIIEQISEFSKRV ILADANLDKVLSAYNKHRDKPIREQAENIIHLFTLTNLGAPAAFKYFDTTIDRKRY TSTKEVLDATLIHQ SITGLYETRIDLSQLGGD (SEQ ID NO:87).

An exemplary reference Cas9 amino acid sequence having an inactivated nuclease domain with a D10A mutation (underlined) is: MDKKYSIGLAIGTNSVGWAVITDEY KVPSKKFKVLGNTDRHSIKKNLIGALLFDSGETAEATRLKRTARRRYTRRKNRIC YLQEIFSNEMAKVDDSFFHRLEESFLVEEDKKHERHPIFGNIVDEVAYHEKYPTIY HLRKKLVDSTDKADLRLIYLALAHMIKFRGHFLIEGDLNPDNSDVDKLFIQLVQT YNQLFEENPINASGVDAKAILSARLSKSRRLENLIAQLPGEKKNGLFGNLIALSLG LTPNFKSNFDLAEDAKLQLSKDTYDDDLDNLLAQIGDQYADLFLAAKNLSDAIL LSDILRVNTEITKAPLSASMIKRYDEHHQDLTLLKALVRQQLPEKYKEIFFDQSKN GYAGYIDGGASQEEFYKFIKPILEKMDGTEELLVKLNREDLLRKQRTFDNGSIPH QIHLGELHAILRRQEDFYPFLKDNREKIEKILTFRIPYYVGPLARGNSRFAWMTRK SEETITPWNFEEVVDKGASAQSFIERMTNFDKNLPNEKVLPKHSLLYEYFTVYNE LTKVKYVTEGMRKPAFLSGEQKKAIVDLLFKTNRKVTVKQLKEDYFKKIECFDS VEISGVEDRFNASLGTYHDLLKIIKDKDFLDNEENEENEDILEDIVLTLTLFEDREMIEE RLKTYAHLFDDKVMKQLKRRRYTGWGRLSRKLINGIRDKQSGKTILDFLKSDGF ANRNFMQLIHDDSLTFKEDIQKAQVSGQGDSLHEHIANLAGSPAIKKGILQTVKV VDELVKVMGRHKPENIVIEMARENQTTQKGQKNSRERMKRIEEGIKELGSQILKE HPVENTQLQNEKLYLYYLQNGRDMYVDQELDINRLSDYDVDHIVPQSFLKDDSI DNKVLTRSDKNRGKSDNVPSEEVVKKMKNYWRQLLNAKLITQRKFDNLTKAER GGLSELDKAGFIKRQLVETRQITKHVAQILDSRMNTKYDENDKLIREVKVITLKS KLVSDFRKDFQFYKVREINNYHHAHDAYLNAVVGTALIKKYPKLESEFVYGDY KVYDVRKMIAKSEQEIGKATAKYFFYSNIMNFFKTEITLANGEIRKRPLIETNGET GEIVWDKGRDFATVRKVLSMPQVNIVKKTEVQTGGFSKESILPKRNSDKLIARK KDWDPKKYGGFDSPTVAYSVLVVAKVEKGKSKKLKSVKELLGITIMERSSFEKN PIDFLEAKGYKEVKKDLIIKLPKYSLFELENGRKRMLASAGELQKGNELALPSKY VNFLYLASHYEKLKGSPEDNEQKQLFVEQHKHYLDEIIEQISEFSKRVILADANLD KVLSAYNKHRDKPIREQAENIIHLFTLTNLGAPAAFKYFDTTIDRKRYTSTKEVLD ATLIHQ SITGLYETRIDLSQLGGD (SEQ ID NO:88).

An exemplary reference Cas9 amino acid sequence having an inactivated nuclease domain with a H840A mutation (underlined) is: MDKKYSIGLDIGTNSVGWAVITDEY KVPSKKFKVLGNTDRHSIKKNLIGALLFDSGETAEATRLKRTARRRYTRRKNRIC YLQEIFSNEMAKVDDSFFHRLEESFLVEEDKKHERHPIFGNIVDEVAYHEKYPTIY HLRKKLVDSTDKADLRLIYLALAHMIKFRGHFLIEGDLNPDNSDVDKLFIQLVQT YNQLFEENPINASGVDAKAILSARLSKSRRLENLIAQLPGEKKNGLFGNLIALSLG LTPNFKSNFDLAEDAKLQLSKDTYDDDLDNLLAQIGDQYADLFLAAKNLSDAIL LSDILRVNTEITKAPLSASMIKRYDEHHQDLTLLKALVRQQLPEKYKEIFFDQSKN GYAGYIDGGASQEEFYKFIKPILEKMDGTEELLVKLNREDLLRKQRTFDNGSIPH QIHLGELHAILRRQEDFYPFLKDNREKIEKILTFRIPYYVGPLARGNSRFAWMTRK SEETITPWNFEEVVDKGASAQSFIERMTNFDKNLPNEKVLPKHSLLYEYFTVYNE LTKVKYVTEGMRKPAFLSGEQKKAIVDLLFKTNRKVTVKQLKEDYFKKIECFDS VEISGVEDRFNASLGTYHDLLKIIKDKDFLDNEENEENEDILEDIVLTLTLFEDREMIEE RLKTYAHLFDDKVMKQLKRRRYTGWGRLSRKLINGIRDKQSGKTILDFLKSDGF ANRNFMQLIHDDSLTFKEDIQKAQVSGQGDSLHEHIANLAGSPAIKKGILQTVKV VDELVKVMGRHKPENIVIEMARENQTTQKGQKNSRERMKRIEEGIKELGSQILKE HPVENTQLQNEKLYLYYLQNGRDMYVDQELDINRLSDYDVDAIVPQSFLKDDSI DNKVLTRSDKNRGKSDNVPSEEVVKKMKNYWRQLLNAKLITQRKFDNLTKAER GGLSELDKAGFIKRQLVETRQITKHVAQILDSRMNTKYDENDKLIREVKVITLKS KLVSDFRKDFQFYKVREINNYHHAHDAYLNAVVGTALIKKYPKLESEFVYGDY KVYDVRKMIAKSEQEIGKATAKYFFYSNIMNFFKTEITLANGEIRKRPLIETNGET GEIVWDKGRDFATVRKVLSMPQVNIVKKTEVQTGGFSKESILPKRNSDKLIARK KDWDPKKYGGFDSPTVAYSVLVVAKVEKGKSKKLKSVKELLGITIMERSSFEKN PIDFLEAKGYKEVKKDLIIKLPKYSLFELENGRKRMLASAGELQKGNELALPSKY VNFLYLASHYEKLKGSPEDNEQKQLFVEQHKHYLDEIIEQISEFSKRVILADANLD KVLSAYNKHRDKPIREQAENIIHLFTLTNLGAPAAFKYFDTTIDRKRYTSTKEVLD ATLIHQ SITGLYETRIDLSQLGGD (SEQ ID NO:89).

In some embodiments, Cas9 variants containing mutations other than D10A and H840A and lacking nuclease activity are provided herein. Such variants include, without limitation, include other amino acid substitutions at D10 and H840, or other substitutions within the Cas9 nuclease domains. In some embodiments, a Cas9 variant can have one or more amino acid additions or deletions (e.g., one, two, three, four, five, six, seven, eight, nine, 10, 10 to 20, 20 to 40, 40 to 50, or 50 to 100 additions or deletions) as compared to a reference Cas9 sequence (e.g., the sequence set forth in SEQ ID NO:86. It is noted, for example, that Cas9 has two separate nuclease domains that allow it to cut both strands of a double-stranded DNA. These are referred to as the “RuvC” and “HNH” domains. Each includes several active site metal-chelating residues. In the RuvC domain, the metal-chelating residues are D10, E762, H983, and D986, while in the HNH domain, the metal-chelating residues are D839, H840, and N863. Mutation of one or more of these residues (e.g., by substituting an alanine for the natural amino acid) may convert Cas9 into a nickase, while mutating one residue from each domain can result in a nuclease-dead and nickase-dead Cas9.

The Cas9 sequences used in the fusion polypeptides provided herein also can be based on natural or engineered Cas9 molecules from organisms such as Corynebacterium ulcerans (NCBI Refs: NC_015683 and NC_017317), C. diphtheria (NCBI Refs: NC_016782 and NC_016786), Spiroplasma syrphidicola (NCBI Ref: NC_021284), Prevotella intermedia (NCBI Ref: NC_017861), Spiroplasma taiwanense (NCBI Ref: NC_021846), Streptococcus iniae (NCBI Ref: NC_021314), Belliella baltica (NCBI Ref: NC_018010), Psychroflexus torquis (NCBI Ref: NC_018721), Streptococcus thermophilus (NCBI Ref: YP_820832), Listeria innocua (NCBI Ref: NP_472073), Campylobacter jejuni (NCBI Ref: YP_002344900), Neisseria meningitidis (NCBI Ref: YP_002342100), and Francisella novicida. RNA-guided nucleases that have similar activity to Cas9 but are from other types of CRISPR/Cas systems, such as Acidaminococcus sp. or Lachnospiraceae bacterium ND2006 Cpf1 (see, e.g., Yamano et al., Cell 165(4):949-962, 2016; and Dong et al., Nature 532(7600):522-526, 2016) also can be used in fusion polypeptides with APOBEC deaminases.

The domains within APOBEC-Cas9 fusion polypeptides provided herein can be positioned in any suitable configuration. In some embodiments, for example, the APOBEC portion can be coupled to the N-terminus of the Cas9 portion, either directly or via a linker. Alternatively, the APOBEC portion can be fused to the C-terminus of the Cas9 portion, either directly or via a linker. In some cases, the APOBEC portion can be fused within an internal loop of Cas9. Suitable linkers include, without limitation, an amino acid or a plurality of amino acids (e.g., five to 50 amino acids, 10 to 20 amino acids, 15 to 25 amino acids, or 25 to 50 amino acids, such as (GGGGS)ₙ (SEQ ID NO:90), (G)n, (EAAAK)ₙ (SEQ ID NO:91), (GGS)ₙ, a SGSETPGTSESATPES (SEQ ID NO:92) motif (see, e.g., Guilinger et al., Nat Biotechnol 32(6):577-582, 2014), an (XP)ₙ motif, or a combination thereof, where n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30). Suitable linkers also include organic groups, polymers, and chemical moieties. Useful linker motifs also are described elsewhere (see, e.g., Chen et al., Adv Drug Deliv Rev 65(10):1357-1369, 2013). When included, a linker can be connected to each domain via a covalent bond, for example.

Additional components that may be present in the fusion polypeptides provided herein include, such as one or more NLSs, cytoplasmic localization sequences, export sequences (e.g., a nuclear export sequence), or sequence tags that are useful for solubilization, purification, or detection of the fusion protein. Suitable localization signal sequences and sequences of protein tags include, without limitation, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Fusion polypeptides also can include other functional domains, such as, without limitation, a domain from the bacteriophage UGI protein that is a universal inhibitor of uracil DNA glycosylase enzymes (UNG2 in human cells; see, e.g., Di Noia and Neuberger, Nature 419(6902):43-48, 2002) that can prevent the deaminated cytosine (DNA uracil) from being repaired by cellular base excision repair (see, e.g., Komor et al., Nature 533(7603):420-424, 2016; Wang et al., Cell Research 27:1289-1292, 2017; and Mol et al., Cell 82:701-708, 1995).

APOBEC-Cas9-gRNA editosome systems can be used for targeted DNA editing, where the gRNA molecule (or molecules, if both the crRNA and tracrRNA are used) targeted to a particular sequence (e.g., in a genome or in an extrachromosomal plasmid) act to direct the Cas9 portion of an APOBEC-Cas9 fusion polypeptide to the target sequence, permitting the APOBEC portion of the fusion to modify a particular cytosine residue at the desired sequence.

Thus, this document provides methods for using APOBEC-Cas9-gRNA editosome systems (e.g., A3H-Cas9-gRNA editosomes) to generate targeted modifications within cellular DNA sequences. The methods can include contacting a target nucleic acid with an APOBEC-Cas9 fusion polypeptide in the presence of one or more gRNA molecules. In some embodiments, the methods can include transforming or transfecting a cell (e.g., a bacterial, plant, or animal cell) with (i) a first nucleic acid encoding an APOBEC-Cas9 fusion polypeptide, and (ii) a second nucleic acid containing a crRNA sequence and a tracrRNA sequence (or a gRNA sequence) targeted to a DNA sequence of interest. Such methods also can include maintaining the cell under conditions in which nucleic acids (i) and (ii) are expressed.

After a nucleic acid is contacted with an APOBEC-Cas9 fusion polypeptide and gRNA, or after a cell is transfected or transformed with an APOBEC-Cas9 fusion and a gRNA, or with one or more nucleic acids encoding the fusion and the CRISPR RNA, any suitable method can be used to determine whether mutagenesis has occurred at the target site. In some embodiments, a phenotypic change can indicate that a change has occurred the target site. PCR-based methods also can be used to ascertain whether a target site contains a desired mutation.

When a first nucleic acid encoding an APOBEC-Cas9 fusion polypeptide and a second nucleic acid containing a crRNA and a trRNA (or a gRNA) are used, the first and second nucleic acids can be included within a single construct, or in separate constructs. Thus, while in some cases it may be most efficient to include sequences encoding the APOBEC-Cas9 polypeptide, the crRNA, and the tracrRNA in a single construct (e.g., a single vector), in other cases first nucleic acid and the second nucleic acid can be present in separate nucleic acid constructs (e.g., separate vectors). In some embodiments, the crRNA and the tracrRNA also can be in separate nucleic acid constructs (e.g., separate vectors). Again, a “vector” is a replicon, such as a plasmid, phage, or cosmid, into which another DNA segment may be inserted so as to bring about the replication of the inserted segment.

The fusion polypeptides described herein, nucleic acids encoding the polypeptides, and compositions containing the polypeptides or nucleic acids, can be administered to a cell or to a subject (e.g., a human, a non-human mammal such as a non-human primate, a rodent, a sheep, a goat, a cow, a cat, a dog, a pig, or a rabbit, an amphibian, a reptile, a fish, or an insect) in order to specifically modify a targeted DNA sequence. In some cases, the targeted sequence can be selected based on its association with a particular clinical condition or disease, and the administration can be aimed at treating the clinical condition or disease. The term “treating” refer to reversal, alleviation, delaying the onset, or inhibiting the progress of the condition or disease, or one or more symptoms of the condition or disease. In some cases, administration can occur after onset of the clinical condition or disease (after one or more symptoms of the condition have developed, for example, or after the disease has been diagnosed). In some cases, however, administration may occur in the absence of symptoms, such that onset or progression of the clinical condition or disease is prevented or delayed. This may be the case when the subject is identified as being susceptible to the condition, for example, or when the subject has been previously treated for the condition and symptoms have resolved, but recurrence is possible.

In some embodiments, the methods provided herein can be used to introduce a point mutation into a nucleic acid by deaminating a target cytosine. For example, the targeted deamination of a particular cytosine may correct a genetic defect (e.g., a genetic defect is associated with a clinical condition or disease). In some embodiments, the methods provided herein can be used to introduce a deactivating point mutation into a sequence encoding a gene product associated with a clinical condition or disease (e.g., an oncogene). In some cases, for example, a deactivating mutation can create a premature stop codon in a coding sequence, resulting in the expression of a truncated gene product that may not be functional, or may lack the normal function of the full-length protein.

In some embodiments, the methods provided can be used to restore the function of a dysfunctional gene. For example, the APOBEC-Cas9 fusion polypeptides described herein can be used in vitro or in vivo to correct a disease-associated mutation (e.g., in cell culture or in a subject). Thus, this document provides methods for treating subjects identified as having a clinical condition or disease that is associated with a point mutation. Such methods can include administering to a subject an APOBEC-Cas9 fusion polypeptide, or a nucleic acid encoding an APOBEC-Cas9 fusion polypeptide, in an amount effective to correct the point mutation or to introduce a deactivating mutation into the sequence associated with the disease. The disease can be, without limitation, a proliferative disease, a genetic disease, or a metabolic disease.

To target an APOBEC-Cas9 fusion polypeptide to a target site (e.g., a site having a point mutation to be edited), the APOBEC-Cas9 fusion can be co-expressed with a crRNA and tracrRNA, or a gRNA, that allows for Cas9 binding and confers sequence specificity to the APOBEC-Cas9 fusion polypeptide. Suitable gRNA sequences typically include guide sequences that are complementary to a nucleotide sequence within about 50 (e.g., 25 to 50, 40 to 50, 40 to 60, or 50 to 75) nucleotides upstream or downstream of the target nucleotide to be edited.

In some embodiments, a reporter system can be used to detect activity of the fusion proteins described herein. See, for example, the luciferase-based assay described in US 2016/0304846, in which deaminase activity leads to expression of luciferase. US 2016/0304846 also describes a reporter system utilizing a reporter gene that has a deactivated start codon. In this reporter system, successful deamination of the target permits translation of the reporter gene.

It is to be noted that, while the examples provided herein relate to APOBEC-Cas9 fusions, the use of other DNA-targeting molecules is contemplated. Thus, for example, a variant APOBEC polypeptide can be coupled to a DNA-targeting domain from a polypeptide such as a meganuclease (e.g., a wild-type or variant protein of the homing endonuclease family, such as those belonging to the dodecapeptide family (LAGLIDADG; SEQ ID NO:93), a transcription activator-like (TAL) effector protein, or a zinc-finger (ZF) protein. Such proteins and their characteristics, function, and use are described elsewhere. See, e.g., WO 2004/067736/Porteus, Nature 459:337-338, 2009; Porteus and Baltimore, Science 300:763, 2003; Bogdanove et al., Curr Opin Plant Biol 13:394-401, 2010; and Boch et al., Science 326(5959):1509-1512, 2009.

The invention will be further described in the following examples, which do not limit the scope of the invention described in the claims.

### EXAMPLES

### Example 1—Materials and Methods

Plasmids for Expression in E. coli:

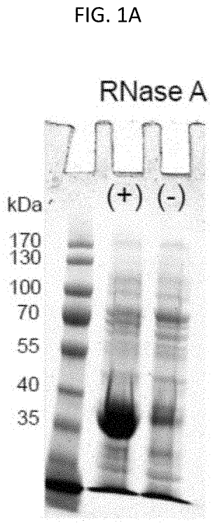

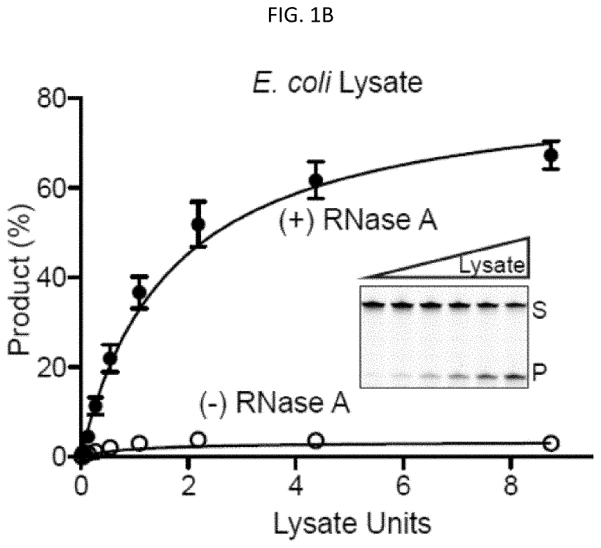

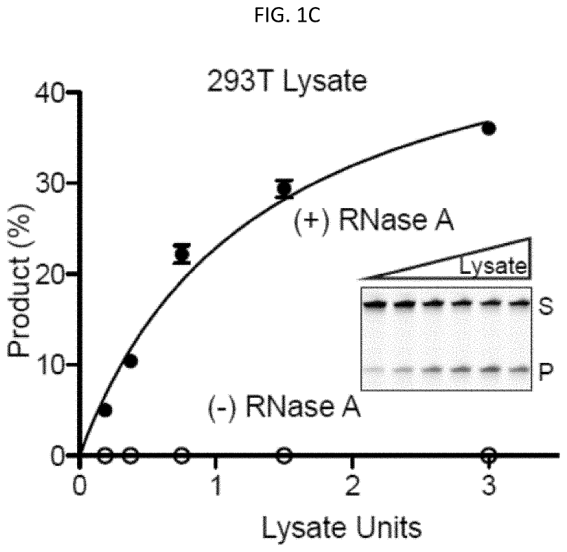

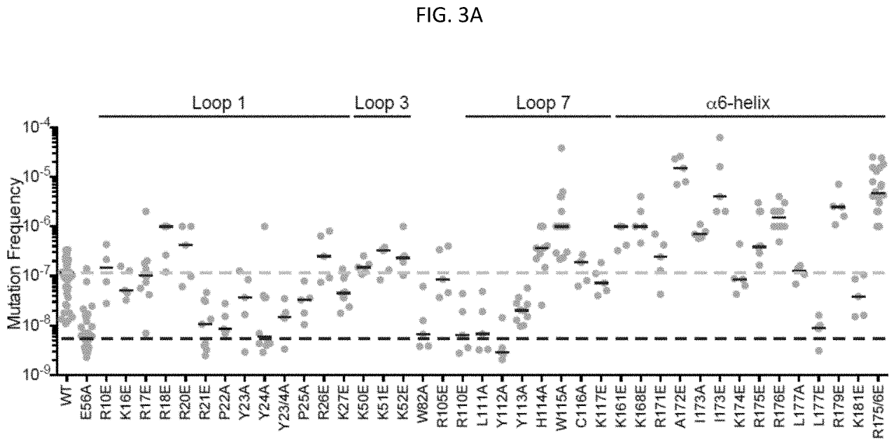

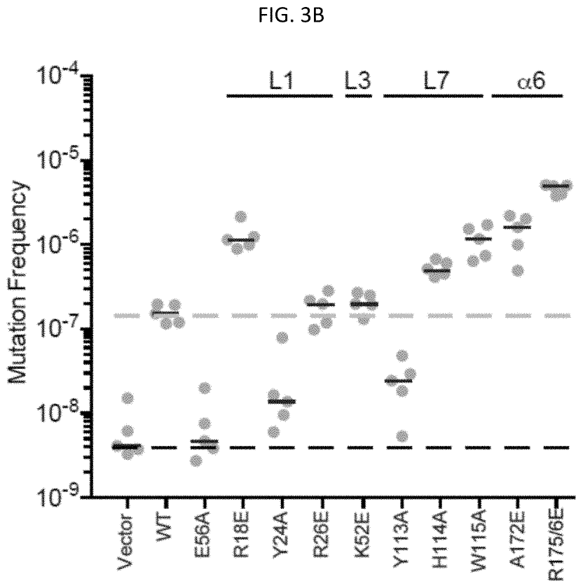

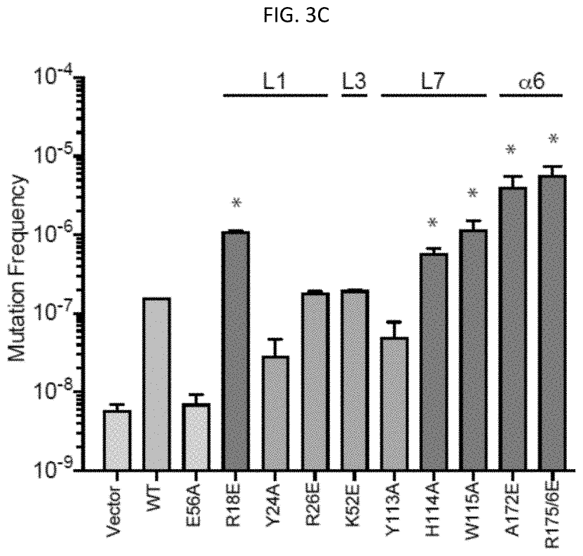

6×His-SUMO-A3H was generated by PCR amplifying A3H HapII 1-183 cDNA (GENBANK®: ACK77775.1) from pcDNA3.1-A3H (Hultquist et al., J Virol 85:11220-11234, 2011) using primers 5′-gcgcGGTCTCTAGG TGGCGGCGGCATGGCTCTGTTAACAGCCG (SEQ ID NO:94) and 5′-gcgcTCTAG ATTAGGACTGCTTTATCCTC (SEQ ID NO:95). The PCR product was digested with BsaI and XbaI and ligated into similarly digested pE-SUMO (Life Sensors). This forward primer also encodes a triple Gly linker. A large panel of mutant derivatives was made by site-directed mutagenesis and verified by Sanger sequencing (sequences of forward primers are shown in TABLE 1 (reverse primers were the complement of the forward primers). The collection of pE-SUMO based constructs was used for experiments in FIGS. 1A, 1B, and 3.

His-tagged mCherry(1-232)-3×Ala linker-A3H-HapII(1-183) was generated by overlap PCR using primers 5′-GGATCCGAATTCGCTGGAAGTTCTGTTCCAGGG GATGGTGAGCAAGGGCGAGGAGG (SEQ ID NO:96), 5′-CTCCACCGGCGGCATG GACGCCGCTGCCATGGCTCTGTTAACAGCCGAAACATTCC (SEQ ID NO:97), 5′-GGAATGTTTCGGCTGTTAACAGAGCCATGGCAGCGGCGTCCATGCCGCCGGT GGAG (SEQ ID NO:98), and 5′-GCGCGCGGCCGCTCAGGACTGCTTTATCC (SEQ ID NO:99). The PCR product was digested with EcoRI and NotI and ligated into MCS1 of prSFDuet-1 (Novagen). A HRV rhinovirus 3C protease site was engineered upstream of the mCherry ATG start site (sequence included in the forward primer used during overlapping PCR).

The mCherry coding sequence was amplified from pRSETB-mCherry (Shaner et al., Nat Biotechnol 22:1567-1572, 2004) and A3H from 6×-His-SUMO-A3H (above). A3HK52E, A3H-E56A, A3H-E56A-K52E, and A3H-E56A-W115A-R175/6E were made by site-directed mutagenesis and verified by Sanger sequencing.

E. coli-Based Rif^{R} Mutation Experiments:

Experiments were conducted as described elsewhere (Harris et al., Mol Cell 10:1247-1253, 2002; and Shi et al., J Biol Chem 290:28120-28130, 2015), with a minimum of five colonies tested for each condition in each independent experiment. His6-SUMO-A3H constructs were transformed into E. coli C43 (DE3) cells, and single colonies were grown to saturation at 37° C. in 2 mL LB plus ampicillin (100 μg/ml). A 100 μl aliquot of undiluted culture was plated directly onto LB-agar plates containing rifampicin (100 μg/ml) to select Rif^{R} mutants. Cultures were diluted serially in 1×M9 salts (sodium phosphate dibasic, potassium phosphate dibasic, sodium chloride, ammonium chloride) and plated on LB to determine viable cell counts. Mutation frequencies were calculated by dividing the number of colonies on the rifampicin plate by the total number of cells in the overnight culture (total number of colonies on LB plates multiplied by dilution factor). Key Rif^{R} mutation data were quantified by comparing mean±SEM of the median mutation frequency from 3 independent experiments and assessing statistical significance using the Student's t-test.

Plasmids for APOBEC Expression in Human Cell Lines:

Coding sequences of A3H exon 2 and exons 3-5 were amplified from pcDNA3.1-A3H (Hultquist et al., supra) using primer pairs 5′-NNNNGGTACCACCATGGCTCTGTTA ACAG (SEQ ID NO: 100)/5′-AAACATCTCCTGGACTCACCTTGTTTTCAAAGTA GCCTC (SEQ ID NO:101) and 5′-GTCTCCTTTCATCTCAACAGAAAAAGTGCCAT GCGGAAAT (SEQ ID NO: 102)/5′-NNNNGCGGCCGCTCAGGACTGCTTTATCCT (SEQ ID NO:103), respectively. Human HGB2 intron 2 was amplified from an A3Bi containing plasmid (Burns et al., Nature 494:366-370, 2013) using primer pair 5′-GAGGCTACTTTGAAAACAAGGTGAGTCCAGGAGATGTTT (SEQ ID NO: 104)/5′-ATTTCCGCATGGCACTTTTTCTGTTGAGATGAAAGGAGAC (SEQ ID NO:105). The amplified fragments were fused together by overlap extension PCR, and inserted into the KpnI-NotI cloning site of pcDNA3.1(+) (ThermoFisher Scientific). Mutant derivatives of pcDNA3.1-A3Hi were generated using site-directed mutagenesis using the forward primers shown in TABLE 2 (reverse primers were the complement of the forward primers), and verified by Sanger sequencing. Plasmids pEGFP-N3 (Clontech Laboratories), pEGFP-N3-A3Bi-eGFP, and pEGFPN3-A3G-eGFP have been reported in localization and HIV-1 restriction studies described elsewhere (Hultquist et al., supra).

N-terminal mCherry-tagged, intron disrupted A3H HapII (1-183) was generated by overlap PCR using primers 5′-TGATCCGCGGCCGCACCACCATGGTGAGCAA GGGCGAGGA (SEQ ID NO:106) (mCherry Forward), 5′-CGGCTGTTAACAGAGCC ATGGCAGCGGCGTCCATGCCACCGGTGGAGTG (SEQ ID NO: 107) (overlap reverse, mCherry), 5′-CACTCCACCGGTGGCATGGACGCCGCTGCCATGGCTCTG TTAACAGCCG (SEQ ID NO: 108) (overlap forward, A3H HapII), and 5′-ATTAAGCGT ACGTCAGGACTGCTTTATCCTGTCAAG (SEQ ID NO: 109) (A3H HapII reverse). The PCR product was digested with NotI and BsiWI and ligated into a pQCXIH retrovirus-based vector (Clontech Laboratories). Designated mutants were PCR-subcloned from pcDNA3.1 expression constructs (above) using the same set of A3H HapII primers. PCR products were digested with AgeI and BsiWI and subcloned into similarly digested parental vector (pQCXIH-mCherry-A3Hi). Sanger sequencing was used to verify the integrity of all constructs.

APOBEC3HDNA Deamination Experiments:

293T cells were plated in 6-well plates at 400,000 cell density and cultured overnight. The following day, the cells were transfected with 1 μg of each pcDNA3.1-A3Hi expression construct and 50 ng of an eGFP expression plasmid (transfection control). Forty-eight (48) hours later, cells were harvested and resuspended in 200 μL HED buffer (25 mM HEPES pH 7.4, 15 mM EDTA, 1 mM DTT, Roche Complete EDTA-free protease inhibitors, 10% glycerol) and frozen at −80° C. Cells were thawed, vortexed, sonicated for 20 minutes in a water bath sonicator (Branson), and centrifuged 10 minutes at 16,000 g, and then soluble extracts were transferred to a new tube. Half of each extract was treated with RNase A (100 μg/ml) at RT for 1 hour. An oligo master mix containing 1.6 μM 5′-ATTATTATTATT CTAATGGATTTATTTATTTATTTATTTATTT (SEQ ID NO: 110)-fluorescein in HED buffer was made and mixed 1:1 with cell extracts (10 μL). Reactions were gently mixed, spun down, then incubated at 37° C. for 60 minutes, heated to 95° C. for 10 minutes to stop the reactions, then incubated with 0.1 U/rxn UDG for 10 minutes at 37° C. Sodium hydroxide was added to a final concentration of 100 mM and reactions were heated to 95° C. to cleave the DNA at abasic sites. Reactions were mixed with 2×DNA PAGE loading dye (80% formamide, 1×TBE, bromophenol blue, xylene cyanol). Reaction products were separated by 15% denaturing PAGE (or TBE-Urea PAGE) and visualized by scanning on a Typhoon FLA-7000 scanner on fluorescence mode. A fraction of each extract was separated by 12% SDS-PAGE and transferred to low-fluorescence PVDF overnight at 20 V. Membranes were blocked with 4% milk in PBST and then incubated with mouse anti-A3H mAb P1D8 (Refsland et al., PLoS Genet 10:e1004761, 2014) or rabbit anti-β-actin mAb (Cell Signaling: 13E5), at dilutions of 1:1,000 or 1:10,000, respectively. After washing in PBST, membranes were probed with the goat anti-mouse 680 antibody (Life Technologies: A21057) or the goat anti-rabbit 800CW antibody (Li-COR Biosciences: 926-32211), each 1:20,000. Blots were stripped and reprobed with rabbit anti-A3H pAb at 1:1,000 (Novus Biologicals NBP1-91682), followed by Li-COR anti-rabbit 800CW secondary at 1:20,000. Imaging was done with a Li-COR Odyssey.

APOBEC3H Purification from E. coli:

His6×-mCherry-A3H HapII 1-183 was transformed into OneShot BL21(DE3) competent cells (Thermo-Fischer Scientific) and, after overnight incubation, colonies were directly inoculated into 2XYT media containing 50 μg/ml kanamycin. About 30 minutes prior to induction, cultures were supplemented with 100 μM zinc sulfate and cooled to 16° C. Protein expression was induced overnight by addition of 0.5 mM IPTG at ˜1 OD600. Cells were centrifuged at 3800 g for 20 minutes and resuspended in lysis buffer containing 50 mM Tris pH 8, 500 mM NaCl, 5 mM imidazole, lysozyme, and RNase A (10 mg). Cells were then sonicated (Branson Sonifer) and cell debris was removed by centrifugation (13000 g, 45 minutes). The supernatant was added to Talon Cobalt Resin (Clontech), washed extensively with wash buffer (50 mM Tris-HCl pH 8.0, 500 mM NaCl, 5 mM imidazole), and eluted with elution buffer (50 mM Tris-HCl pH 8, 500 mM NaCl, 250 mM imidazole). Fractions containing protein were pooled and dialyzed overnight into buffer containing 50 mM Tris-HCl pH 8, 100 mM NaCl, 5% glycerol, 2 mM DTT, and RNase A (5 mg). The protein was concentrated to ˜5 mL and loaded onto a 5 mL HiTrap MonoQ cartridge (GE Healthcare) equilibrated with wash buffer (50 mM Tris-HCl pH 8.0, 100 mM NaCl). The bound protein was washed with wash buffer and eluted with a linear gradient of high salt buffer (50 mM Tris pH 8.0, 1 M NaCl). The protein eluted between 30-40% high salt buffer. Fractions were collected and purified further with a 26/600 S200 gel filtration column (GE Healthcare) using a buffer containing 20 mM Tris-HCl pH 8.0 and 500 mM NaCl. Fractions containing purified protein were pooled, mixed with 5 mM DTT, and concentrated to 25 mg/ml. The final purified protein had an OD260/280 measurement greater than 1, indicating bound nucleic acid. The wild-type mCherry-A3H, mCherry-A3HK52E (a fully functional derivative), mCherry-A3H-E56A (a catalytic mutant), and mCherry-A3H-E56A-K52E proteins were purified using the above protocol. The RNA binding defective mCherry-A3H mutant (W115A, R175E, R176E plus E56A to prevent bacterial toxicity) was purified by resuspending cell pellets in 50 mM Hepes pH 7.5, 1 M NaCl, 5 mM Imidazole, 100 μg/ml of RNase A (RNase A is not required for purification of hyperactive mutant but was added to clarify cell lysates). The cells were lysed, clarified, and hyperactive protein was purified using Talon resin similar to wild-type mCherry-A3H, with the exception that the wash buffer contained 50 mM Hepes pH 7.5, 1 M NaCl, 5 mM Imidazole, and the elution buffer contained 50 mM Hepes pH 7.5, 500 mM NaCl, and 250 mM Imidazole. The protein was immediately purified further by a 26/600 S200 size exclusion column using the same buffer as wild-type mCherry-A3H proteins. The hyperactive A3H proteins were particularly prone to aggregation, so they typically were prepared fresh before use.

APOBEC3H Proteinase K, RNase A, and DNase I Treatment Experiments:

A 4 μL aliquot of each mCherry-A3H protein solution (mCherry-A3Hwt, mCherry-A3H K52E, mCherry-A3H-E56A, mCherry-A3H-E56A-K52E; ˜80 μg total protein in buffers above) was diluted in 14 μL nuclease-free water and 2 μL of Proteinase K (Roche: 03115828001). The tubes were then incubated for 15 minutes each at 45° C., 55° C., and 65° C., and then 10 minutes at 95° C. to inactivate Proteinase K. Then, 2 uL was taken from each tube and incubated with 1) 8 uL nuclease free water, 2) 1 uL DNase I in 7 uL Buffer RDD from Qiagen (79254), or 3) 1 mg/mL RNase A (Sigma: R5503) in 7 uL nuclease free water for 15 minutes at room temperature. Each sample was then mixed with 10 μL 2×DNA PAGE loading dye and half of the reaction was loaded on a 20% TBE-7M Urea gel and run at 10 W. The gel was stopped when the bromophenol blue band was about 99% of the way down the gel. The gel was stained with SYBR-Gold and imaged on a Typhoon FLA-7000 (GE Healthcare Life Sciences). The IDT oligo length standard (51-05-1501; 0.25 μL) was loaded to estimate size (about 25 ng/oligo).

APOBEC3H Crystallization, X-Ray Data Collection and Structural Determination:

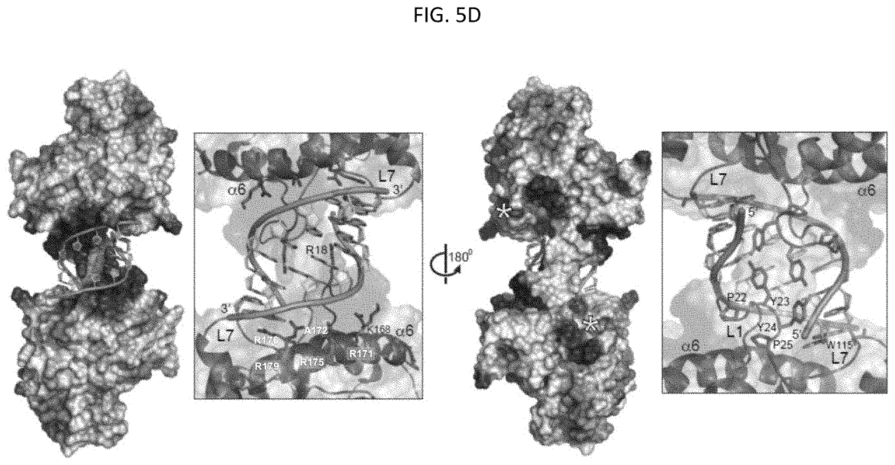

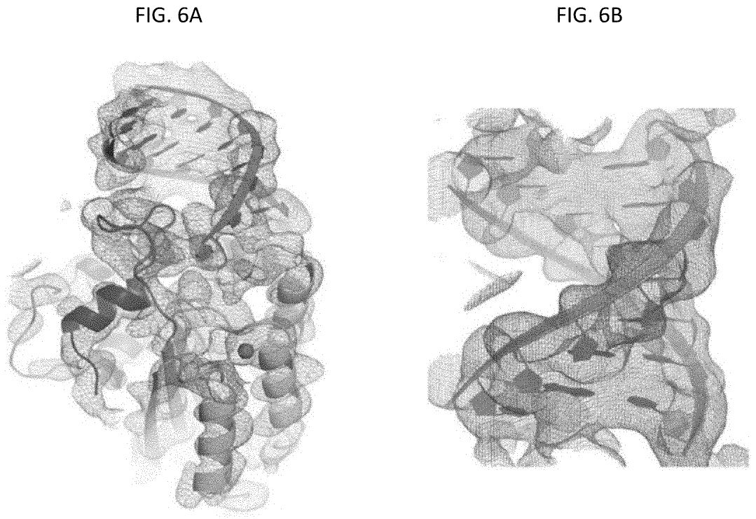

Prior to crystallization, 10 mM DTT and trace amounts of human rhinovirus 3C protease was added to an aliquot of protein diluted to 20 mg/ml to remove the 6×His tag. mCherry-A3H K52E was crystallized using sitting drop vapor diffusion in a solution containing 18% PEG 3350 (v/v) and 300 mM ammonium iodide. The crystal trays were incubated at 18° C. and protein crystals appeared overnight and grew to full size within 2-5 days. The crystals were preserved in cryoprotectant solution containing 20% PEG3350, 300 mM ammonium iodide, and 20% glycerol. The mCherry-A3H-E56A-K52E protein was crystallized using similar methodology. Initial x-ray diffraction data were collected at the Advanced Photon Source NE-CAT 24 ID-E beamline at the wavelength of 0.979 Å. The datasets were processed using HKL2000 (Otwinowski and Minor, 1997) and XDS (Kabsch, 2010), initially in the high-symmetry space group P6122 with good merging statistics. However, structure solution by molecular replacement was unsuccessful despite exhaustive attempts using mCherry and various A3 structures as search models. Even after reprocessing the data in a lower-symmetry space group P31, molecular replacement calculation with mCherry as the search model, which has 100% sequence homology with the mCherry tag used in crystallization and accounts for >50% of macromolecular content in the unit cell, did not yield any solution, implying less than ideal occupancy of mCherry in the crystal. Subsequently, high-multiplicity x-ray diffraction data were collected at the NE-CAT 24-ID-C beamline at the zinc K-edge wavelength of 1.282 Å. Single-wavelength anomalous dispersion (SAD) phasing using SHELX C/D/E (Sheldrick, 2010) and PHENIX (Adams et al., Acta Crystallogr D Biol Crystallogr 66:213-221, 2010) in space group P6122 produced an electron density map that clearly showed secondary structure elements of A3H and a segment of RNA double-helix, which has a deep and narrow major groove and a shallow and wide minor groove (FIG. 6). The A3H and RNA molecules were built into the experimental map using COOT (Emsley et al., Acta Crystallogr D Biol Crystallogr 66:486-501, 2010). The mFo-DFc difference map after initial refinement with phase-restraints against the SAD-derived phases revealed a β-barrel structure corresponding to partial mCherry. Including mCherry in the refinement lowered the Rfree by 1.0% even though mCherry overlapped with its symmetry related molecule. However, the Rfree stayed above 40%. Refinement in a lower symmetry space group P3112 resulted in significant improvement of Rfree and refining with a twin operator “−h,−k,l” increased the Rfree. Therefore refinement was continued in the space group P3112 without twinning. Subsequent iterative model building using COOT and refinement with PHENIX suite resulted in the final Rwork/Rfree of 35.3/36.2%. The slightly high R-factors are likely due to the uncertain location of mCherry. The model quality during refinement was checked constantly by combining the SAD-derived and model-based phases to minimize possible model bias. The asymmetric unit contains two A3H molecules, two RNA strands, and two copies of mCherry with occupancy refined to 0.5. However, two RNA double helices (mixture poly-A paired with poly-U) were modeled in the asymmetric unit, each with half-occupancy, to represent double-stranded RNA molecules comprising mixed populations of unknown complementary sequences and following crystallographic 2-fold symmetry as an ensemble. In spite of the positional ambiguity, mCherry contributed significantly to the total diffraction, as refinement with the mCherry removed led to ˜5% increase in Rfree. To address overlaps between symmetry-related mCherry molecules, space group choices P31 or P1, with half of the mCherry molecules unmodeled, were considered. However, refinement in these space groups led to significantly poorer Rfree. Therefore, P3112 would be the top choice for the space group, in which case mCherry does not have full occupancy. Fluorescent proteins related to mCherry tend to have alternative packing modes in crystals, as reported elsewhere (Pletnev et al., Acta Crystallogr D Biol Crystallogr 65:906-912, 2009; and Pletnev et al., Acta Crystallogr D Biol Crystallogr 70:31-39, 2014). For mCherry-A3H-E56A-K52E, refinement was performed as described above, using the refined mCherry-A3H-K52E structure as the starting model. The summary of data collection and model refinement statistics is shown in TABLE 4. Images were produced using PYMOL (www.pymol.org).

Fluorescent Microscopy Experiments:

About 10,000 293T or 8,000 HeLa cells were plated into a 96-well CellBIND microplate (Corning) and allowed to adhere overnight. Cells were transfected with 100 ng of the indicated untagged A3H construct. For localization controls, 100 ng of A3Bi-eGFP, A3G-eGFP, or eGFP expression vectors (above) were transfected. Forty-eight (48) hours post-transfection, cells were washed twice with PBS and fixed with 4% paraformaldehyde (20 minutes at RT). Cells were then washed twice with cold PBS and permeabilized with 0.5% Triton-X100 (10 minutes, 4° C.). Cells were washed once with cold PBS, incubated in blocking buffer (4% BSA, 5% goat serum, in PBS) for 1 hour at RT, and then stained with 1:100 rabbit anti-A3H pAb (Novus Biologicals, NBP1-91682), or with a rabbit anti-A3B mAb (5210-87-13) at 1:500 that also cross-reacts with A3G (Leonard et al., Cancer Res 75:4538-4547, 2015). Cells were washed with cold PBS and stained with 1:1000 goat anti-rabbit Alexa Flour 594 (ThermoFisher Scientific, A11037) for 1 hour at RT. After additional washing, cells were incubated in PBS containing 0.1% DAPI. Images were collected at 40× magnification using an EVOS FL Color microscope (ThermoFisher Scientific), and quantified using ImageJ software. Individual cells were scored and grouped into three categories (nuclear, whole cell, or cytoplasmic). Subcellular compartments were defined based on DAPI staining (nucleus), anti-A3G-eGFP staining (cytoplasm), and eGFP localization (whole cell) The nuclear:cytoplasmic A3 ratio was calculated by dividing the pixel density of the nuclear compartment (marked by DAPI) divided by the pixel density of cytoplasmic compartment (i.e., the cytoplasmic signal is the whole cell A3 signal minus the nuclear A3 signal). The nuclear:cytoplasmic A3 ratio for each individual cell (n>25 per condition) was graphed with Prism 6.0 (GraphPad Software). Finally, mean ratios±SEM were superimposed over each dot plot, and statistical significance was determined using a 2-tailed Student's t-test. A complementary series of experiments was done with N-terminally mCherry-tagged A3Hi constructs (constructions above) in 293T and HeLa cells as above, except live cells were imaged and mCherry fluorescence was detected using the RFP EVOS light cube (531/40 nm excitation; 593/40 nm emission).

HIV-1 Packaging and Restriction Experiments:

The HIV-1IIIB VifX26X27 provirus expression construct is described elsewhere (Albin et al., J Virol 84:10209-10219, 2010; and Hache et al., Curr Biol 18:819-824, 2008). Analogous tandem stop codons were introduced into codons 26 and 27 of the full-length HIV-1 molecular clone pLAI.2 (Peden et al., Virology 185:661-672, 1991) (AIDS Reagent Program 2532) by subcloning the vif-vpr region into pJET1.2, performing site-directed mutagenesis (Stratagene), and shuttling the vif-vpr region back into the original vector using PshAI and SalI restriction sites. HIV-1 packaging and restriction experiments were done by transfecting 50% confluent 293T cells (TransIt, Mirus) with 1 μg Vif-deficient provirus plasmid and an amount of each A3 expression construct or vector control indicated in the relevant figure legend. After 48 hours of incubation, viral supernatants were cleared of cells by filtration (0.45 μm) and used to infect CEM-GFP cells to monitor infectivity via flow cytometry.

Cell and viral particle lysates were prepared for immunoblotting as follows. Cells were pelleted, washed, and then lysed with 2.5× Laemmli sample buffer. Virus-containing supernatants were filtered and pelleted via centrifugation through a 20% sucrose cushion, then lysed in 2.5× Laemmli sample buffer. Lysates were then subjected to SDS-PAGE followed by protein transfer to PVDF using a Bio-Rad Criterion system. Membranes were probed with a mouse anti-A3H mAb P1D8 (Refsland et al. 2014, supra), a rabbit anti-A3H pAb (NBP1-91682, Novus), an anti-HIV-1 p24/CA mAb (AIDS Reagent Program 3537), and a mouse anti-tubulin mAb (Covance). Secondary antibodies were goat anti-rabbit IRdye 800CW (Li-COR 926-32211) and goat anti-mouse Alexa Fluor 680 (Molecular Probes A21057). Membranes were imaged using a Li-COR Odyssey instrument, and images were prepared for presentation using Image J. For Vif-null HIV-1IIIB dose response experiments, the mean viral infectivity±SD from 3 biologically independent experiments was determined and quantification of the levels of A3H packaging relative to WT was done for relevant RNA binding mutants. First, viral particle A3H and p24 levels and cellular A3H and tubulin levels were quantified. Second, the relative packaging efficiency (RPE) was calculated for each condition using the following formula: RPE: (VLP A3H/p24)/(cellular A3H/TUB). Lastly, normalization was done by dividing the RPE value for each RNA binding mutant by the RPE value for WT A3H, and statistical significance was determined from three biologically independent experiments by comparing mean RPE±SEM using a one-sided Student's t-test.

Apobec-Cas9 Construction:

The rat APOBEC 1-Cas9n-UGI-NLS construct (BE3) was provided by David Liu (Komor et al., supra). APOBEC3 cDNA sequences were amplified using the primers listed in TABLE 3 and high-fidelity PCR using previously validated plasmids as templates. The resulting PCR products were cleaved with NotI and XmaI and used to replace rat APOBEC 1 in BE3 (the NotI site was in the multiple cloning site, and the XmaI site was in the XTEN linker). The sequences cloned into BE3 included the following:

Human APOBEC3C:
(SEQ ID NO: 137)
MNPQIRNPMKAMYPGTFYFQFKNLWEANDRNETW
LCFTVEGIKRRSVVSWKTGVFRNQVDSETHCHAERCFLSWFCDDILSPNT
KYQVTWYTSWSPCPDCAGEVAEFLARHSNVNLTIFTARLYYFQYPCYQEG
LRSLSQEGVAVEIMDYEDFKYCWENFVYNDNEPFKPWKGLKTNFRLLKRR
LRESLQ
Human APOBEC3D:
(SEQ ID NO: 138)
MNPQIRNPMERMYRDTFYDNFENEPILYGRSYTWL
CYEVKIKRGRSNLLWDTGVFRGPVLPKRQSNHRQEVYFRFENHAEMCFLS
WFCGNRLPANRRFQITWFVSWNPCLPCVVKVTKFLAEHPNVTLTISAARL
YYYRDRDWRWVLLRLHKAGARVKIMDYEDFAYCWENFVCNEGQPFMPWYK
FDDNYASLHRTLKEILRNPMEAMYPHIFYFHFKNLLKACGRNESWLCFTM
EVTKHHSAVFRKRGVFRNQVDPETHCHAERCFLSWFCDDILSPNTNYEVT
WYTSWSPCPECAGEVAEFLARHSNVNLTIFTARLCYFWDTDYQEGLCSLS
QEGASVKIMGYKDFVSCWKNFVYSDDEPFKPWKGLQTNFRLLKRRLREIL
Q
Human AP OBEC3F:
(SEQ ID NO: 139)
MKPHFRNTVERMYRDTFSYNFYNRPILSRRNTVWL
CYEVKTKGPSRPRLDAKIFRGQVYSQPEHHAEMCFLSWFCGNQLPAYKCF
QITWFVSWTPCPDCVAKLAEFLAEHPNVTLTISAARLYYYWERDYRRALC
RLSQAGARVKIMDDEEFAYCWENFVYSEGQPFMPWYKFDDNYAFLHRTLK
EILRNPMEAMYPHIFYFHFKNLRKAYGRNESWLCFTMEVVKHEISPVSWK
RGVFRNQVDPETHCHAERCFLSWFCDDILSPNTNYEVTWYTSWSPCPECA
GEVAEFLARHSNVNLTIFTARLYYFWDTDYQEGLRSLSQEGASVEIMGYK
DFKYCWENFVYNDDEPFKPWKGLKYNFLFLDSKLQEILE
Human APOBEC3H:
(SEQ ID NO: 81)
MALLTAETFRLQFNNKRRLRRPYYPRKALLCYQLTP
QNGSTPTRGYFENKKKCHAEICFINEIKSMGLDETQCYQVTCYLTWSPCS
SCAWELVDFIKAHDHLNLRIFASRLYYHWCKPQQDGLRLLCGSQVPVEVM
GFPEFADCWENFVDHEKPLSFNPYKMLEELDKNSRAIKRRLDRIKQS

eGFP Reporter:

The dual fluorescent HIV-based parental vector (Lenti-CMV-mCherry-T2A-eGFP; St. Martin et al., Nucl Acids Res 46(14):e84, 2018, doi.org/10.1093/nar/gky332) was used as the basis for single base editing reporters. In each instance, wild-type eGFP sequence was replaced with a mutant eGFP fragment created by overlapping extension high-fidelity PCR with PHUSION® DNA polymerase (NEB) and the primers listed in TABLE 3. Full-length PCR products were gel purified, digested with XhoI and KpnI, and ligated into a similarly cut parental vector. The resulting L202, L138, and Y93 single base editing reporters were confirmed by diagnostic restriction digestions and Sanger sequencing.

Quantification and Statistical Analysis:

Key Rif^{R} mutation data were quantified by comparing mean±SEM of the median mutation frequency from 3 independent experiments and assessing statistical significance using the Student's t-test. X-Ray data collection and refinement statistics for crystal structures are shown in TABLE 4. A3H localization and packaging experiments were quantified and assessed statistically as described above.

Data and software availability: The atomic coordinates and structure factors for mCherry-K52E and mCherry-E56A-K52E were deposited in the Protein Data Bank with accession codes 6BOB and 6BBO, respectively. All original gel and microscopy images from are deposited in Mendeley (dx.doi.org/10.17632/6mdtnv4kjb.1).

TABLE 1

| pESUMO | | |
|---|---|---|
| A3H HapII | | |
| 1-183 | | SEQ ID |
| Mutations | Site-Directed Forward Primer (5′-3′) | NO: |
| R10E | CTGTTAACAGCCGAAACATTCGAGTTACAGTTTAACAACAAGCG | 141 |
| K16E | CAGTTTAACAACGAGCGCCGCCTCAG | 142 |
| R17E | CTTACAGTTTAACAACAAGGAACGCCTCAGAAGGCCTTAC | 143 |
| R18E | CAGTTTAACAACAAGCGCGAACTCAGAAGGCCTTACTAC | 144 |
| R20E | CAAGCGCCGCCTCGAAAGGCCTTACTAC | 145 |
| R21E | CAACAAGCGCCGCCTCAGAGAACCTTACTACCCGAGGAAG | 146 |
| P22A | CGCCTCAGAAGGGCTTACTACCCGAG | 147 |
| Y23A | CTCAGAAGGCCTGCCTACCCGAGGAAG | 148 |
| Y24A | GAAGGCCTTACGCCCCGAGGAAGGC | 149 |
| P25A | GAAGGCCTTACTACGCCAGGAAGGCCCTCTTG | 150 |
| R26E | CAGAAGGCCTTACTACCCGGAAAAGGCCCTCTTGTGTTAC | 151 |
| K27E | CCTTACTACCCGAGGGAAGCCCTCTTGTGTTACC | 152 |
| K50E | GGCTACTTTGAAAACGAGAAAAAGTGCCATGC | 153 |
| K51E | CTACTTTGAAAACAAGGAAAAGTGCCATGCGG | 154 |
| K52E | CTACTTTGAAAACAAGAAAGAGTGCCATGCGGAAATTTGC | 155 |
| E56A | CATCGGCTCGTATAATGTG | 156 |
| R105E | GACCATCTGAACCTGGAGATCTTCGCCTCCCG | 157 |
| R110E | CGCATCTTCGCCTCCGAGCTGTACTACCACTG | 158 |
| L111A | CTTCGCCTCCCGCGCATACTACCACTGG | 159 |
| Y112A | CCTCCCGCCTGGCTTACCACTGGTGC | 160 |
| Y113A | CCCGCCTGTACGCGCACTGGTGCAAG | 161 |
| H114A | CGCCTGTACTACGCCTGGTGCAAGC | 162 |
| W115A | CCTGTACTACCACGCATGCAAGCCCCAG | 163 |
| C116A | GTACTACCACTGGGCAAAGCCCCAGCAG | 164 |
| K117E | CTACCACTGGTGCGAACCCCAGCAGGAC | 165 |
| K161E | CCTTCAACCCCTATGAGATGTTAGAGGAGC | 166 |
| K168E | GATGTTAGAGGAGCTAGATGAAAACAGTCGAGCCATAAAG | 167 |
| R171E | GCTAGATAAAAACAGTGAAGCCATAAAGCGACGGC | 168 |
| A172E | CTAGATAAAAACAGTCGAGAAATAAAGCGACGGCTTGAC | 169 |
| I173A | GATAAAAACAGTCGAGCCGCAAAGCGACGGCTTGACAG | 170 |
| I173E | GATAAAAACAGTCGAGCCGAAAAGCGACGGCTTGACAG | 171 |
| K174E | GATAAAAACAGTCGAGCCATAGAACGACGGCTTGACAGGATAAAG | 172 |
| R175E | GAGCCATAAAGGAACGGCTTGACAG | 173 |
| R175E R176E | CAGTCGAGCCATAAAGGAGGAGCTTGACAGGATAAAGC | 174 |
| R176E | GTCGAGCCATAAAGCGAGAGCTTGACAGGATAAAG | 175 |
| L177A | GCCATAAAGCGACGGGCTGACAGGATAAAGC | 176 |
| L177E | GAGCCATAAAGCGACGGGAAGACAGGATAAAGCAGTC | 177 |
| R179E | CATAAAGCGACGGCTTGACGAGATAAAGCAGTCCTAATC | 178 |
| K181E | CGGCTTGACAGGATAGAGCAGTCCTAATCTAG | 179 |

TABLE 2

| pCDNA3.1 | | |
|---|---|---|
| A3Hi HapII | | |
| 1-183 | | SEQ ID |
| Mutations | Site-Directed Forward Primer (5′-3′) | NO: |
| R10E | CTGTTAACAGCCGAAACATTCGAGTTACAGTTTAACAACAAGCG | 180 |
| K16E | CAGTTTAACAACGAGCGCCGCCTCAG | 181 |
| R17E | CTTACAGTTTAACAACAAGGAACGCCTCAGAAGGCCTTAC | 182 |
| R18E | CAGTTTAACAACAAGCGCGAACTCAGAAGGCCTTACTAC | 183 |
| R20E | CAAGCGCCGCCTCGAAAGGCCTTACTAC | 184 |
| R21E | CAACAAGCGCCGCCTCAGAGAACCTTACTACCCGAGGAAG | 185 |
| P22A | CGCCTCAGAAGGGCTTACTACCCGAG | 186 |
| Y23A | CTCAGAAGGCCTGCCTACCCGAGGAAG | 187 |
| Y24A | GAAGGCCTTACGCCCCGAGGAAGGC | 188 |
| P25A | GAAGGCCTTACTACGCCAGGAAGGCCCTCTTG | 189 |
| R26E | CAGAAGGCCTTACTACCCGGAAAAGGCCCTCTTGTGTTAC | 190 |
| K27E | CCTTACTACCCGAGGGAAGCCCTCTTGTGTTACC | 191 |
| K50E | GGCTACTTTGAAAACGAGAAAAAGTGCCATGC | 192 |
| K51E | GTCTCCTTTCATCTCAACAGGAAAAGTGCCATGCGGAAATTTGC | 193 |
| K52E | CTACTTTGAAAACAAGAAAGAGTGCCATGCGGAAATTTGC | 194 |
| E56A | CATCGGCTCGTATAATGTG | 195 |
| R110E | CGCATCTTCGCCTCCGAGCTGTACTACCACTG | 196 |
| L111A | CTTCGCCTCCCGCGCATACTACCACTGG | 197 |
| Y112A | CCTCCCGCCTGGCTTACCACTGGTGC | 198 |
| Y113A | CCCGCCTGTACGCGCACTGGTGCAAG | 199 |
| H114A | CGCCTGTACTACGCCTGGTGCAAGC | 200 |
| W115A | CCTGTACTACCACGCATGCAAGCCCCAG | 201 |
| C116A | GTACTACCACTGGGCAAAGCCCCAGCAG | 202 |
| K117E | CTACCACTGGTGCGAACCCCAGCAGGAC | 203 |
| K161lE | CCTTCAACCCCTATGAGATGTTAGAGGAGC | 204 |
| K168E | GATGTTAGAGGAGCTAGATGAAAACAGTCGAGCCATAAAG | 205 |
| R171E | GCTAGATAAAAACAGTGAAGCCATAAAGCGACGGC | 206 |
| A172E | CTAGATAAAAACAGTCGAGAAATAAAGCGACGGCTTGAC | 207 |
| I173A | GATAAAAACAGTCGAGCCGCAAAGCGACGGCTTGACAG | 208 |
| I173E | GATAAAAACAGTCGAGCCGAAAAGCGACGGCTTGACAG | 209 |
| K174E | GATAAAAACAGTCGAGCCATAGAACGACGGCTTGACAGGATAAAG | 210 |
| R175E | GAGCCATAAAGGAACGGCTTGACAG | 211 |
| R175E R176E | CAGTCGAGCCATAAAGGAGGAGCTTGACAGGATAAAGC | 212 |
| R176E | GTCGAGCCATAAAGCGAGAGCTTGACAGGATAAAG | 213 |
| L177A | GCCATAAAGCGACGGGCTGACAGGATAAAGC | 214 |
| L177E | GAGCCATAAAGCGACGGGAAGACAGGATAAAGCAGTC | 215 |
| R179E | CATAAAGCGACGGCTTGACGAGATAAAGCAGTCCTGAGC | 216 |
| K181E | GCTTGACAGGATAGAGCAGTCCTGAGC | 217 |

TABLE 3

| | | SEQ ID |
|---|---|---|
| Primer Target | Primer Sequence | NO: |
| Full-length A3Bi cloning | AGATCCGCGGCCGCGCCGCCACCATGAATCCACAGATCAG | 218 |
| primer forward | AAATCCGATGG | |
| Full-length A3Bi cloning | TGAGGTCCCGGGAGTCTCGCTGCCGCTGTTTCCCTGATTCT | 219 |
| primer reverse | GGAGAATGGCC | |
| A3C cloning primer | AGATCCGCGGCCGCGCCGCCACCATGAATCCACAGATCAG | 220 |
| forward | AAACCCGATGA | |
| A3C cloning primer | TGAGGTCCCGGGAGTCTCGCTGCCGCTCTGGAGACTCTCC | 221 |
| reverse | CGTAGCCTTCTT | |
| A3D cloning primer | AGATCCGCGGCCGCGCCGCCACCATGAATCCACAGATCAG | 222 |
| forward | AAATCCGATGG | |
| A3D cloning primer | TGAGGTCCCGGGAGTCTCGCTGCCGCTCTGGAGAATCTCC | 223 |
| reverse | CGTAGCCTTCTT | |
| A3F cloning primer | AGATCCGCGGCCGCGCCGCCACCATGAAGCCTCACTTCAG | 224 |
| forward | AAACACAGTGG | |
| A3F cloning primer | TGAGGTCCCGGGAGTCTCGCTGCCGCTCTCGAGAATCTCC | 225 |
| reverse | TGCAGCTTGCTG | |
| A3G cloning primer | AGATCCGCGGCCGCGCCGCCACCATGAAGCCTCACTTCAG | 226 |
| forward | AAACACAGTGG | |
| A3G cloning primer | TGAGGTCCCGGGAGTCTCGCTGCCGCTGTTTTCCTGATTCT | 227 |
| reverse | GGAGAATGGCC | |
| A3H-I and A3H-II | AGATCCGCGGCCGCGCCGCCACCATGGCTCTGTTAACAGC | 228 |
| cloning primer forward | CGAAACATTCCG | |
| A3H-i and A3H-II | TGAGGTCCCGGGAGTCTCGCTGCCGCTTCAGGACTGCTTT | 229 |
| cloning primer reverse | ATCCTGTCAAGC | |
| Y93H Mutation | CCATGCCCGAAGGTCACGTACAGGAGCGGACCATCTTC | 230 |
| Forward | | |
| Y93H Mutation | GAAGATGGTCCGCTCCTGTACGTGACCTTCGGGCATGG | 231 |
| Reverse | | |
| L138S Mutation | GGACGGCAACATTTCAGGGCACAAGCTGGA | 232 |
| Forward | | |
| L138S Mutation | TCCAGCTTGTGCCCTGAAATGTTGCCGTCC | 233 |
| Reverse | | |
| L202S Mutation | CGACAACCACTATTCAAGTACCCAGTCGGCCCTGA | 234 |
| Forward | | |
| L202S Mutation | TCAGGGCCGACTGGGTACTTGAATAGTGGTTGTCG | 235 |
| Reverse | | |
| GFP Y93H gRNA | ACACCCCGAAGGTCACGTACAGGAG | 236 |
| forward | | |
| GFP Y93H gRNA | AAAACCTCCTGTACGTGACCTTCGGG | 237 |
| reverse | | |
| GFP L138S gRNA | ACACCCAACATTTCAGGGCACAAGCG | 238 |
| forward | | |
| GFP L138S gRNA | AAAACGCTTGTGCCCTGAAATGTTGG | 239 |
| reverse | | |
| GFP L202S gRNA | ACACCCCACTATTCAAGTACCCAGTG | 240 |
| forward | | |
| GFP L202S gRNA | AAAACACTGGGTACTTGAATAGTGGG | 241 |
| reverse | | |
| CloneJET Sequencing | CGACTCACTATAGGGAGAGCGGC | 242 |
| Forward Primer | | |
| CloneJET Sequencing | AAGAACATCGATTTTCCATGGCAG | 243 |
| Reverse Primer | | |
| L138 Miseq Forward No | ACACTCTTTCCCTACACGACGCTCTTCCGATCTTCGAGCTG | 244 |
| C | AAGGGCATCGAC | |
| L138 Miseq Forward | ACACTCTTTCCCTACACGACGCTCTTCCGATCTCTCGAGCT | 245 |
| One C | GAAGGGCATCGAC | |
| L138 Miseq Forward | ACACTCTTTCCCTACACGACGCTCTTCCGATCTCCTCGAGC | 246 |
| Two C | TGAAGGGCATCGAC | |
| L138 Miseq Reverse No | GTGACTGGAGTTCAGACGTGTGCTCTTCCGATCTTAGACGT | 247 |
| C | TGTGGCTGTTGTA | |
| L138 Miseq Reverse | GTGACTGGAGTTCAGACGTGTGCTCTTCCGATCTGTAGAC | 248 |
| One C | GTTGTGGCTGTTGTA | |
| L138 Miseq Reverse | GTGACTGGAGTTCAGACGTGTGCTCTTCCGATCTGGTAGA | 249 |
| Two C | CGTTGTGGCTGTTGTA | |
| L202 Miseq Forward No | ACACTCTTTCCCTACACGACGCTCTTCCGATCTCCATCGGC | 250 |
| C | GACGGCCCCGTG | |
| L202 Miseq Forward | ACACTCTTTCCCTACACGACGCTCTTCCGATCTCCCATCGG | 251 |
| One C | CGACGGCCCCGTG | |
| L202 Miseq Forward | ACACTCTTTCCCTACACGACGCTCTTCCGATCTCCCCATCG | 252 |
| Two C | GCGACGGCCCCGTG | |
| L202 Miseq Reverse No | GTGACTGGAGTTCAGACGTGTGCTCTTCCGATCTCGCTTCT | 253 |
| C | CGTTGGGGTCTTT | |
| L202 Miseq Reverse | GTGACTGGAGTTCAGACGTGTGCTCTTCCGATCTGCGCTTC | 254 |
| One C | TCGTTGGGGTCTTT | |
| L202 Miseq Reverse | GTGACTGGAGTTCAGACGTGTGCTCTTCCGATCTGGCGCTT | 255 |
| Two C | CTCGTTGGGGTCTTT | |

TABLE 4
Data Collection and Refinement Statistics

| | |
|---|---|
| 6B0B | 6BBO (E56A) |

Data collection

| Resolution range | 49.2-3.28 | (3.54-3.28)* | 81.4-3.45 | (3.78-3.45) |
|---|---|---|---|---|
| Space group | P3₁12 | | P3₁12 | |
| Unit cell a, b, c (Å) | 101.29 101.29 211.22 | | 101.97 101.97 210.00 | |
| Total reflections | 226613 | (21387) | 78256 | (18359) |
| Unique reflections | 19319 | (1672) | 16797 | (3988) |
| Multiplicity | 11.6 | (11.4) | 4.7 | (4.6) |
| Completeness (%) | 99.9 | (99.4) | 99.9 | (99.9) |
| I/σ(I) | 12.6 | (0.7) | 5.5 | (1.1) |
| R-merge | 0.132 | (3.82) | 0.188 | (1.55) |
| R-meas | 0.144 | (4.19) | 0.241 | (1.98) |
| R-pim | 0.043 | (1.23) | 0.111 | (0.92) |
| CC_{1/2} | 1.000 | (0.499) | 0.997 | (0.494) |

Refinement

| | | | | |
|---|---|---|---|---|
| Reflections | 18933 | (1672) | 16395 | (1353) |
| Reflections for | 967 | (109) | 866 | (74) |
| R-free | | | | |

| | | |
|---|---|---|
| R-work (%) | 35.3 | 36.3 |
| R-free (%) | 36.2 | 39.2 |
| # non-H atoms | 6932 | 6934 |
| Macromolecules | 6928 | 6920 |
| Ligands | 2 | 12 |
| Solvent | 2 | N/A |
| Protein residues | 770 | 770 |
| r.m.s.d. | | |
| Bond lengths (Å) | 0.0004 | 0.003 |
| Bond angles (°) | 1.05 | 0.94 |
| Ramachandran plot | | |
| Favored (%) | 92.49 | 95.44 |
| Allowed (%) | 7.51 | 4.56 |
| Outliers (%) | 0.00 | 0.00 |
| Average B-factor | 203.82 | 142.03 |
| (Å²) | | |
| Macromolecules | 203.82 | 142.15 |
| (Å²) | | |
| Ligands (Å²) | 156.83 | 85.32 |
| Solvent (Å²) | 129.89 | N/A |

*Statistics for the highest-resolution shell are shown in parentheses.

### Example 2—RNA Digestion Enhances APOBEC3H Purification and DNA Cytosine Deaminase Activity

Initial attempts to purify His6-SUMO-A3H from E. coli were unsuccessful because most protein failed to bind the affinity resin. To determine whether RNA might be promoting aggregation and preventing the hexahistidine tag from binding, lysates were treated with RNase A. After metal affinity purification, fractions were compared by SDS PAGE. This procedure enabled strong recovery of A3H, but only with RNase A treatment (FIG. 1A). Further, an oligo-based single-stranded DNA deamination activity assay showed that A3H catalytic activity was only detected in E. coli lysates treated with RNase A, suggesting that RNA can inhibit deaminase activity (FIG. 1B). A similar requirement for RNase A was evident in activity assays of A3H expressed in human 293T cells (FIG. 1C).

### Example 3—Identification of Hyperactive APOBEC3H Variants

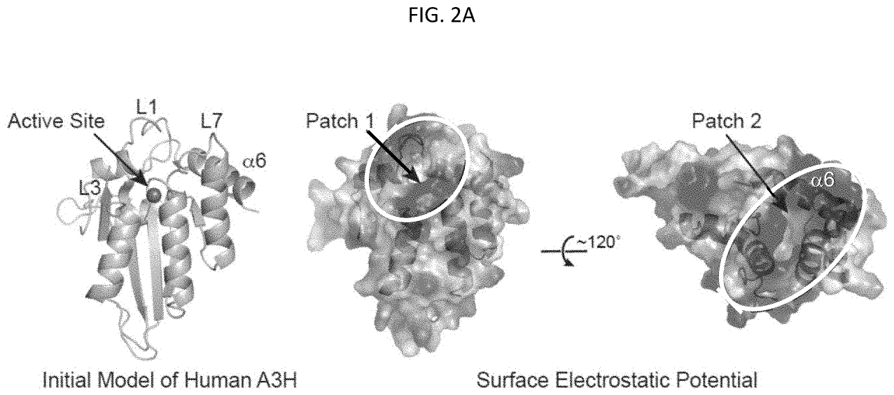

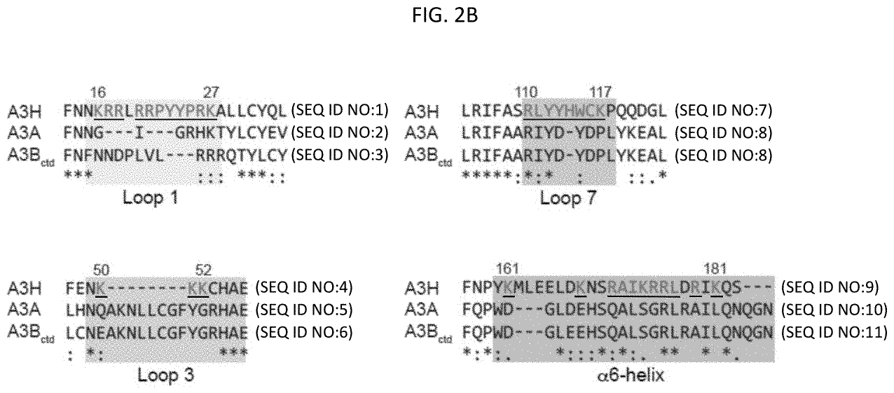

Since RNA can inhibit A3H catalytic activity, it is possible that RNA binds the active site pocket and directly prevents the binding of single-stranded DNA. Alternatively, the RNA binding domain may be distinct and indirectly inhibit DNA deamination activity by forming inhibitory complexes. An A3H structural model was generated to help distinguish between these possibilities and inform mutagenesis experiments (FIG. 2A). A3A/B-ssDNA co-crystal structures demonstrated that loop 1, loop 7, and active site residues define a positively charged region; this region may be essential for A3H to bind single-stranded DNA and potentially also RNA (Kouno et al., Nat Commun 8:15024, 2017; and Shi et al., J Biol Chem 290:28120-28130, 2017). Thus, in this model, the two activities would be inseparable by mutagenesis (patch 1 in FIG. 2A).

Surprisingly, the structural model also predicted a second positively charged region centered on α-helix 6, hereafter referred to as patch 2 (FIG. 2A). Amino acids within this basic patch include Arg171, Lys174, Arg175, Arg176, and Arg179 (FIG. 2B). To determine whether residues located in this basic patch are important for binding RNA, studies were conducted to assess whether substitutions with this site would impair the formation of RNA-inhibited complexes and increase catalytic DNA deaminase activity. A panel of mutant A3H constructs covering both patch 1 and patch 2 was tested in rifampicin-resistance (Rif^{R}) mutation assays, which provide a quantitative measure of DNA mutator activity (Harris et al., Mol Cell 10:1247-1253, 2002). Ala and positively charged Arg and Lys residues were changed to Glu, and other residues (Leu, Ile, Tyr, Trp) were changed to Ala (FIG. 2B). The background Rif^{R} mutation frequency was about 1×10⁻⁸ (catalytic mutant E56A and empty vector controls; lower dashed line in FIGS. 3A and 3B). Wild-type A3H caused a 10-fold increase in the median Rif^{R} mutation frequency (upper dashed line in FIGS. 3A and 3B). Several A3H amino acid substitutions had no significant effect, including several loop 1 and 3 changes (R26E, K50E, K51E, and K52E, which were important for structural studies as described below), whereas other substitutions partially or completely abrogated mutator activity (catalytic E56A, loop 1 R21E, P22A, Y23A, Y24A, and P25A, and loop 7 R110E, L111A, Y112A, and Y113A). The latter group of mutants was most likely defective in DNA deamination.

Strikingly, several substitutions caused much higher Rif^{R} mutation frequencies, including substitutions at predicted loop 1, loop 7, and α6-helix residues (loop 1 R18E and R20E, loop 7 H114A and W115A, and α6-helix R171E, A172E, I173A/E, R175E, R176E, and R179E). In a few instances, combinations of these amino acid substitutions led to mutation frequencies nearly 100-fold higher than wild-type A3H (e.g., R175/6E; quantification in FIG. 3C). One triple mutant combination was toxic to E. coli (W115A plus R175/6E), consistent with the fact that it elicited the highest overall DNA mutator activity. This was the first instance of toxicity indicated for A3H. These hypermutator phenotypes provided strong support for the model in which the RNA binding site in A3H is distinct from the single-stranded DNA binding and catalytic region.

### Example 4—DNA Deaminase Activity of APOBEC3H and Derivatives in Human Cell Extracts

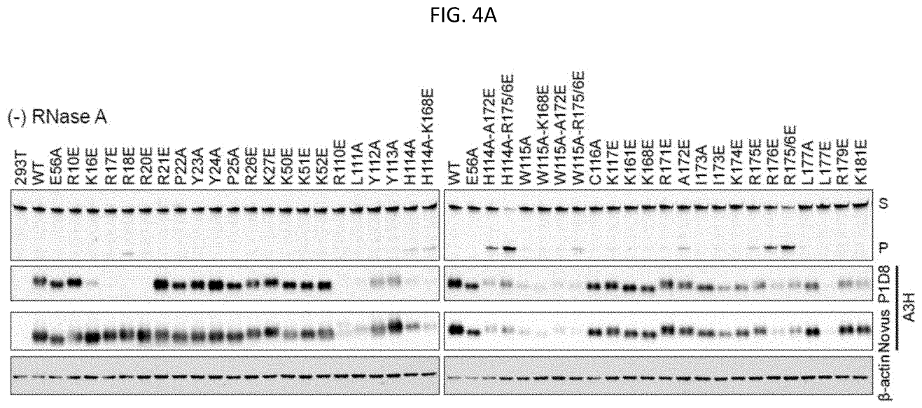

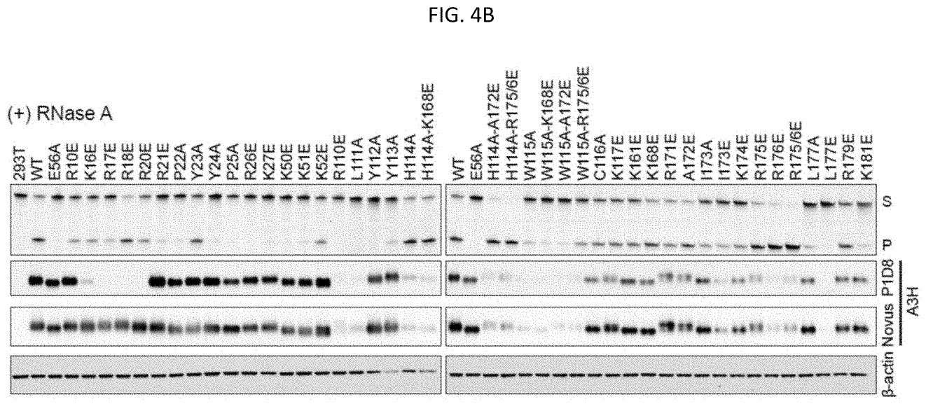

To extend the bacterial mutation results, the majority of the panel of A3H mutant constructs was expressed in human 293T cells and tested for single-stranded DNA cytosine deaminase activity±RNase A treatment. Human 293T cell lysates have no detectable single-stranded DNA cytosine deaminase activity unless transfected with an active APOBEC expression construct (see, e.g., Carpenter et al., J Biol Chem 287:34801-34808, 2012; Stenglein et al., J Virol 82:9591-9599, 2010; and Thielen et al., PLoS Pathog 3:1320-1334, 2007)]. Consistent with the above results, wild-type A3H and most mutant derivatives showed no activity in the absence of RNase A treatment (FIG. 4A, upper panels). In contrast, most of the E. coli hypermutators displayed clear DNA cytosine deaminase activity under these repressive conditions, including loop 1 R18E, loop 7 H114A and W115A, and α6-helix A172E, R175E, and R176E, consistent with a RNA binding defect. Moreover, combinations of activating amino acid substitutions resulted in enzymes with further enhanced DNA deaminase activity (e.g., R175/6E). It was also notable that several of the hypermutators had low steady-state expression levels, suggesting they may be even more active than indicated in this series of comparative biochemical experiments (FIG. 4A, lower panels).

In comparison, RNase A treatment enabled most constructs to elicit DNA cytosine deaminase activity, except those predicted to be defective in binding single-stranded DNA or catalyzing C-to-U deamination (e.g., catalytic E56A, loop 1 R21E, P22A, Y24A, and P25A, and loop 7 R110E, L111A, Y112A, and Y113A; FIG. 4B). Immunoblots of the same cell extracts showed that most constructs, apart from several hyperactive mutants (as above), were expressed at near wild-type A3H levels. The use of two different antibodies enabled analyses of untagged constructs, which overcame issues associated with epitope alterations. For instance, constructs with changes spanning residues Lys16 to Arg20 were detected readily using a rabbit polyclonal reagent, but showed no signal with mouse anti-A3H monoclonal antibody P1D8, consistent with its binding site mapping to the N-terminal 30 residues (Refsland et al. 2014, supra). Overall, the results of the Rif^{R} mutation experiments and in vitro DNA deaminase studies correlated strongly, with several hyperactive mutants clearly standing-out.

### Example 5—Human APOBEC3H Purification and X-Ray Crystal Structure Determination

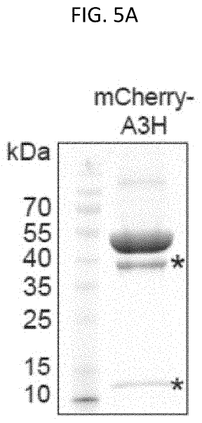

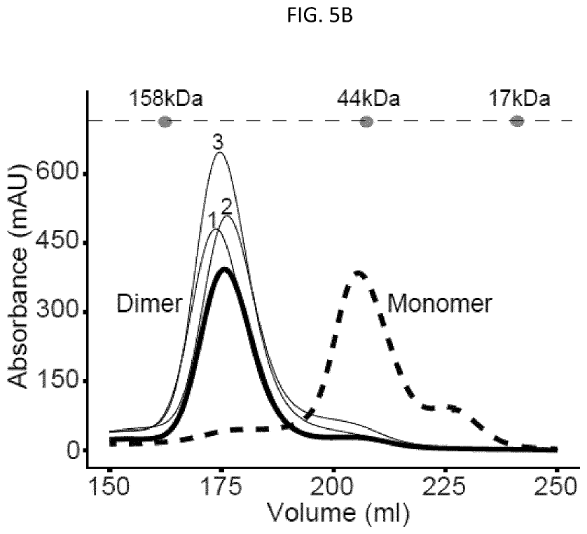

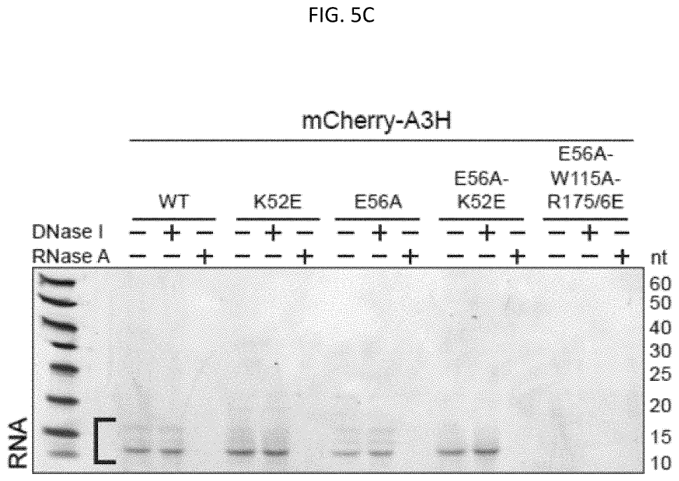

To directly compare the RNA binding activities of A3H and representative hyperactive mutants, His6-mCherry-tagged A3H constructs were expressed in E. coli and metal affinity purification was performed. Again, wild-type A3H, a fully functional derivative (K52E), and a catalytic mutant derivative (E56A) showed requirements for RNase A treatment during purification, whereas hyperactive mutants did not (FIG. 5A). Further purification of wild-type A3H using an ion exchange column and size exclusion chromatography resulted in a ˜100 kDa species that was consistent with dimerization (FIG. 5B). Several derivative proteins (K52E, E56A, and E56A/K52E) had similar profiles and, like wild-type A3H, showed high 260/280 ratios (>1), suggesting that some RNA was still bound. In contrast, hyperactive mutants eluted as monomeric species with low 260/280 ratios (˜0.6), consistent with near-pure protein (e.g., FIG. 5B). To characterize the bound nucleic acid, a panel of purified A3H proteins was proteinase K-treated and the remaining material was heat-denatured, treated with RNase A or DNase I, and analyzed by polyacrylamide gel electrophoresis (FIG. 5C). The resulting gel image revealed a range of short RNase A-sensitive RNA oligomers, with a major species of ˜10 nucleotides long. In contrast, no RNA was apparent in hyperactive enzyme preparations (e.g., FIG. 5C). These results supported a model in which RNA somehow mediates A3H dimerization.

Studies were then conducted to determine the structure of wild-type human A3H and provide a molecular explanation for the aforementioned genetic and biochemical data. During purification from E. coli, it was observed that wild-type A3H was prone to aggregation and precipitation. Multiple N-terminal solubility tags (Sumo, MBP, GST, GFP, and mCherry) were then tested, revealing that mCherry-A3H was soluble but failed to crystallize. The linker region between mCherry and A3H was then optimized, resulting in non-diffracting crystals. As discussed above, extensive portions of the A3H surface are predicted to be charged positively, with Lys and Arg residues projecting into solution (FIG. 2A). Because an excessive concentration of positive charge may hinder crystallization, loop 3 lysine residues were systematically changed to glutamate to help overcome this issue while simultaneously preserving the integrity of patch 2. A single amino acid A3H variant, K52E, was identified as having wild-type activities in the systems described above, indicating fully intact DNA and RNA binding activities (FIGS. 3, 4, 5B, and 5C).

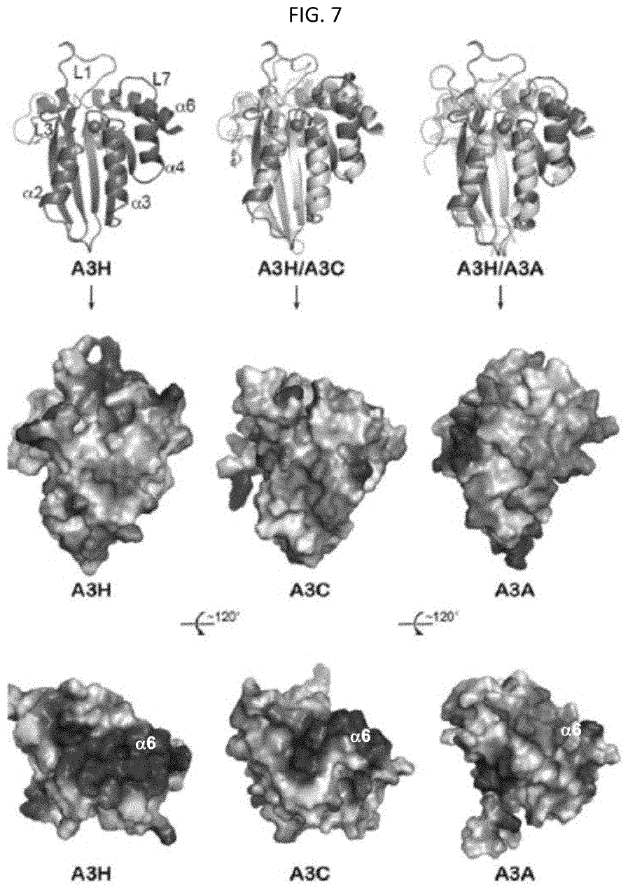

The optimized construct—mCherry-A3H-K52E—was amenable to purification, crystallization, and structure determination. The resulting crystal structure revealed two A3H monomers bridged by a RNA double helix (FIG. 5D, TABLE 4, and FIG. 6). The crystal structure also showed that each A3H monomer has a cytidine deaminase fold with a central 5-strand β-sheet and 6 surrounding α-helices (FIG. 7), that a single zinc ion is coordinated in the active site of each monomer, and that a 7 bp RNA duplex with an additional 1 nucleotide overhang is nestled between the same region of each A3H monomer, anchored by positively charged α6-helix residues (Arg171, Arg175, Arg176, and Arg 179). These residues make direct interactions with the RNA phosphate backbone and encompass a total surface area of ˜650 Å2 (FIG. 5D). Notably, Arg to Glu substitutions at these positions caused increased activity in both the Rif^{R} mutation assay and the in vitro DNA deamination assay, with combinations often eliciting the highest activities (FIGS. 3 and 4). In addition, although Ala172 does not appear to interact directly with the RNA, an A172E substitution yielded a hyperactive protein (RNA binding-defective), likely due to repulsive electrostatic effects.

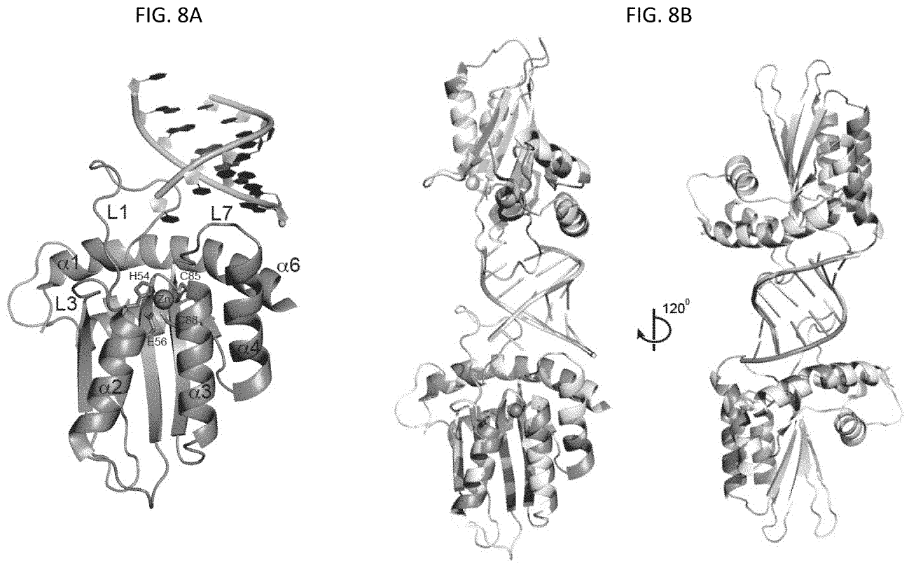

Additional A3H-RNA contacts are made by residues in loops 1 and 7, as indicated by E. coli and in vitro activity data. In particular, Trp 115 of loop 7 is base stacked with the 5′ end of the RNA, and a portion of loop 1, including Arg18, of each monomer projects into the major groove of the RNA helix (180° view in FIG. 5D). Tyr residues in the loop 1 PYYP motif make direct contacts with the RNA phosphate backbone, and a Tyr23 interaction may also facilitate a monomer-monomer contact. Overall, loop 1, loop 7, and α6-helix residues cradle the RNA duplex through extensive interactions. Identical contacts occur in an independent crystallographic data set using a catalytic mutant derivative (mCherry-A3H-K52E-E56A) (TABLE 4 and FIG. 8). These structural results demonstrated a molecular mechanism in which the prime mediator of A3H dimerization is duplex RNA.

### Example 6—RNA-Binding is Required for APOBEC3H Cytoplasmic Localization

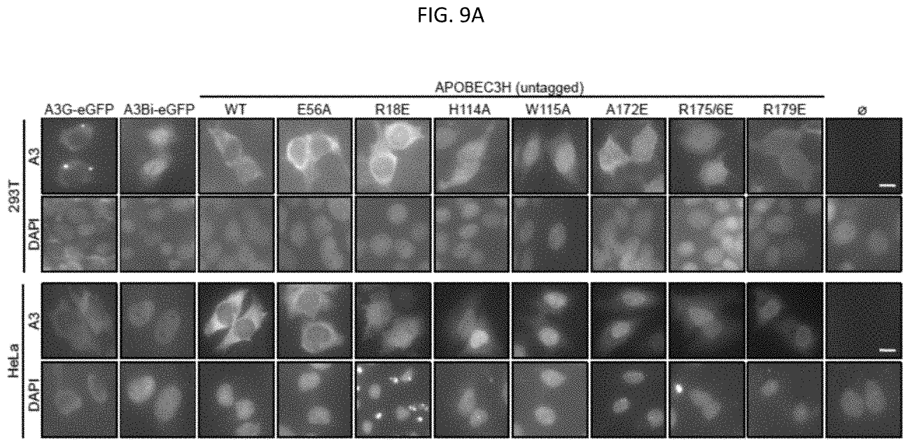

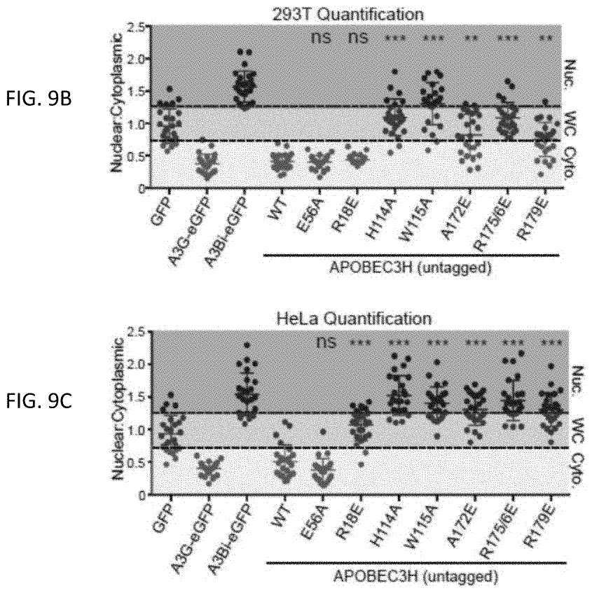

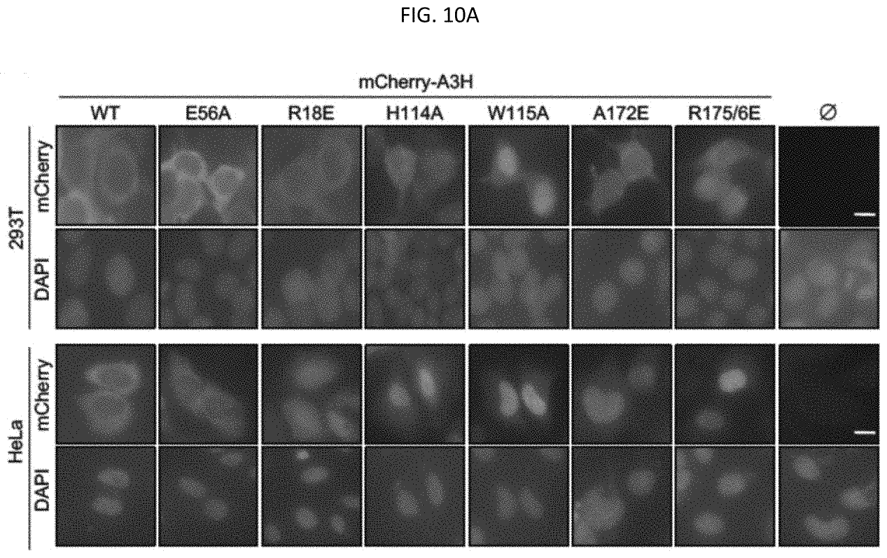

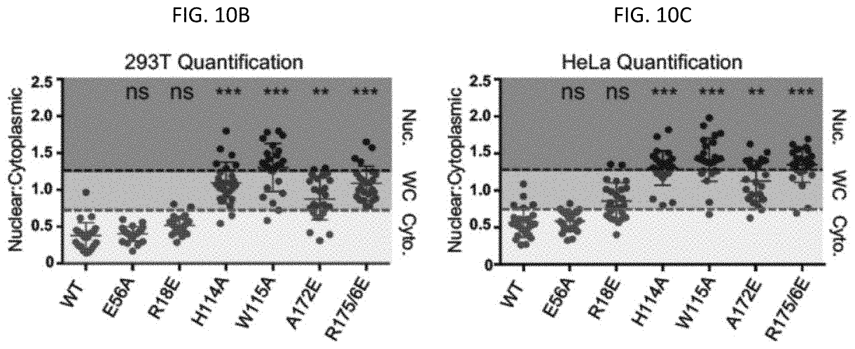

An APOBEC-RNA interaction is thought to have roles in subcellular localization and packaging into HIV-1 particles, which is an essential step in the overall retrovirus restriction mechanism (Harris and Dudley, Virology 479-480C:131-145, 2015; Malim and Bieniasz, Cold Spring Harbor Perspectives in Medicine 2:a006940, 2012; and Simon et al., Nat Immunol 16:546-553, 2015). Studies described elsewhere have shown that A3H is predominantly cytoplasmic, and that this activity is conserved between the human and rhesus macaque enzymes (Hultquist et al., Virol 85:11220-11234, 2011; and Li and Emerman, J Virol 85:8197-8207, 2011). To test whether the RNA binding domain of A3H is required for cytoplasmic localization, a series of fluorescent microscopy experiments was performed in two different cell types (293T and HeLa) with untagged, wild-type human A3H, and with a panel of RNA binding mutants. Cytoplasmic A3G and nuclear A3B were scored in parallel as controls. As expected, wild-type A3H was predominantly cytoplasmic. In contrast, five of six RNA binding domain mutants showed disrupted cytoplasmic localization in 293T cells, and six of six RNA binding domain mutants showed disrupted cytoplasmic localization in HeLa cells (representative images in FIG. 9A and quantification in FIG. 9B). The different localization phenotype of A3H R18E, as well as A172E and R179E, between the two cell types may have been due to a partial loss of RNA binding activity and/or to altered constellations of potential RNA partners. In comparison, A3H H114A, W115A, and R175/6E enzymes were similarly and strongly mislocalized in both cell types, suggesting a complete loss of RNA binding activity. Similar subcellular localization phenotypes were apparent for mCherry-tagged constructs in living 293T and HeLa cells (FIG. 10). Taken together with the aforementioned genetic, biochemical, and structural data, it was concluded that RNA binding is essential for A3H cytoplasmic localization.

Example 7—RNA-Binding is Required for HIV-1 Restriction

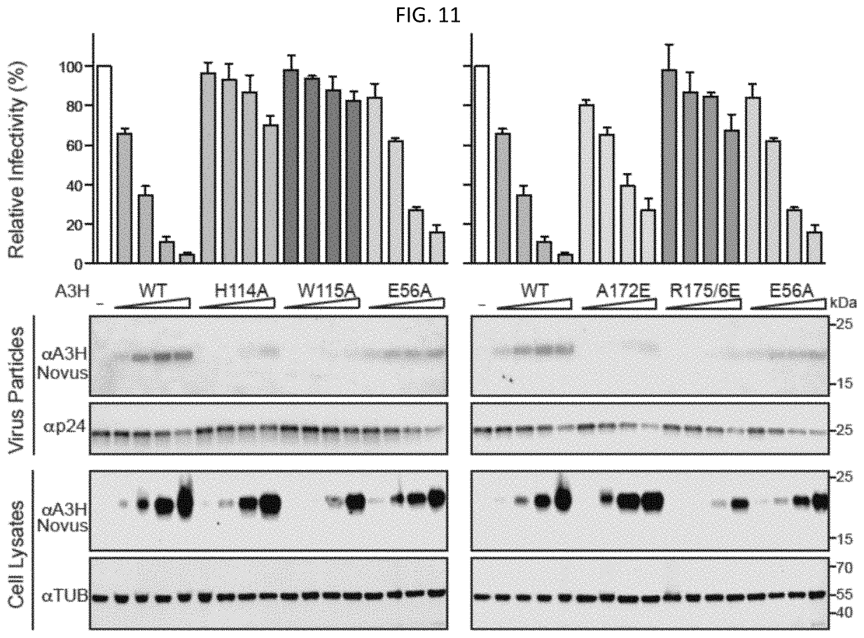

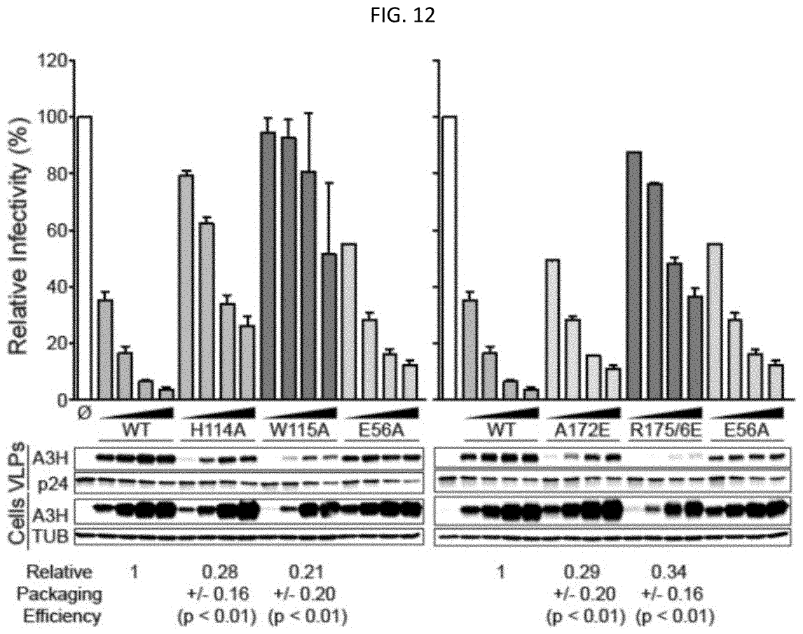

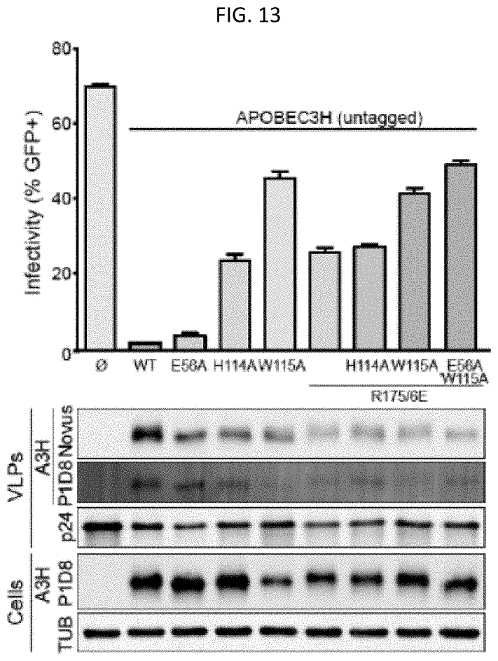

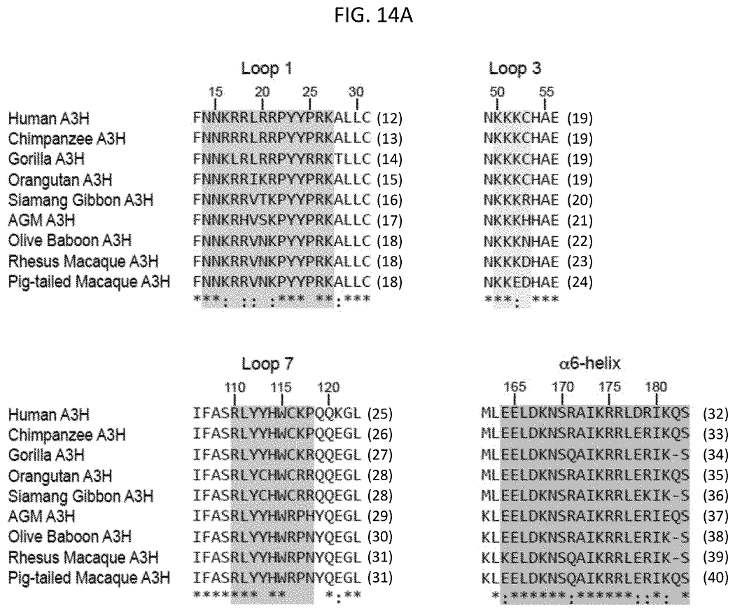

Studies were then conducted to determine whether the RNA binding domain of A3H is required for HIV-1 encapsidation and restriction. Single-cycle infectivity experiments were performed using 293T cells to produce Vif-deficient HIV-1 (LAI) in the presence of wild-type A3H, catalytic mutant E56A, or several of the RNA binding-defective constructs used above in localization studies (FIG. 11). As expected, increasing amounts of wild-type A3H caused large reductions in viral infectivity (>10-fold at highest concentrations) compared to the vector only control, and the catalytic mutant E56A exerted a similar but slightly lower suppressive effect. This result suggested that most of the anti-HIV-1 activity of A3H was independent of DNA deaminase activity. In contrast, the RNA binding-deficient A3H mutants elicited considerably less than wild-type antiviral activity. For instance, in comparison to the vector only control, the H114A and W115A single mutants and the R175/6E double mutant had very little antiviral activity (less than 2-fold reduction at highest protein concentrations tested). Interestingly, the A172E single mutant had partially (not fully) compromised antiviral activity, suggesting retention of some RNA binding activity and consistent with the partial mislocalization phenotype described above. Corresponding immunoblots indicated that most of the compromised antiviral activity of these RNA binding mutants was due to defective packaging into viral particles. A different HIV-1 isolate (IIIB) showed very similar results (FIG. 12). Moreover, the defective packaging and antiviral activities of H114A, W115A, and R175/6E were not exacerbated by combining these amino acid changes, suggesting that each was already completely defective in RNA binding activity (FIG. 13), further consistent with these mutants showing the strongest mislocalization phenotypes in FIGS. 9 and 10. Thus, it was concluded that A3H binds duplex RNA as part of a HIV-1 packaging mechanism, and that this fundamental activity is required for virus restriction.

Example 8—Conservation of the A3H-RNA Binding Mechanism

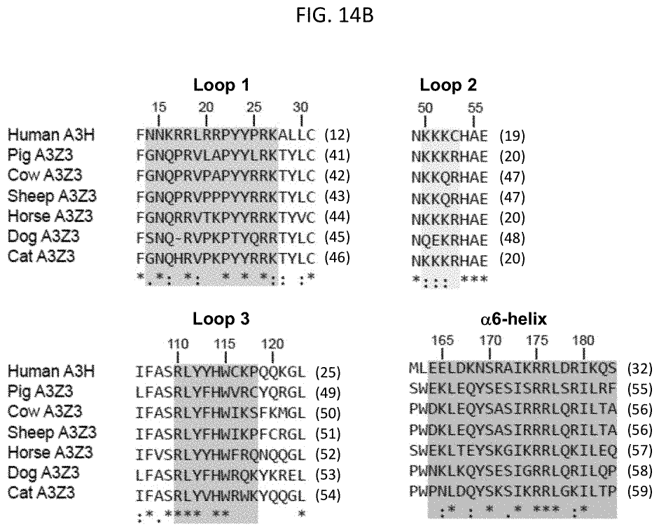

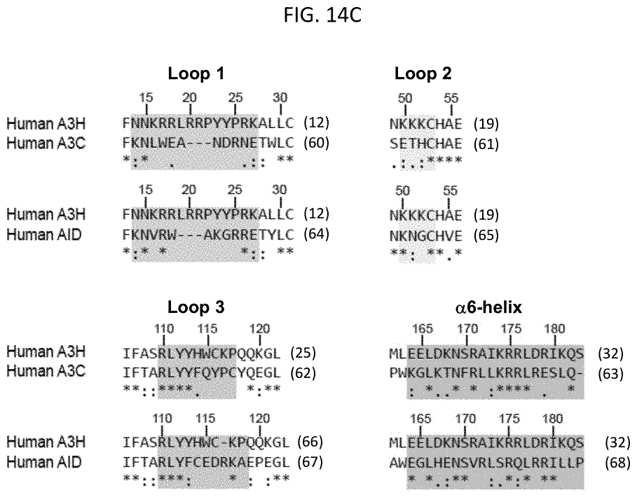

Amino acid alignments indicate that the human A3H-RNA binding mechanism is conserved among primate A3H enzymes, and likely also for homologous mammalian Z3-type deaminases (FIGS. 14A and 14B). In particular, residues homologous to human A3H loop 1 Arg 18, loop 7 His114 and Trp115, and α-helix 6 Arg171, Ala172, Arg175, Arg176, and Arg179 are present in all primate A3H enzymes. Mammalian A3Z3 homologs also have near-identical α-helix 6 and loop 7 residues, as well as positively charged residues situated toward the N-terminal end of loop 1. More distantly related APOBEC family members may also have similar duplex RNA binding mechanisms. For instance, human A3C (Z2-type enzyme) and human AID have analogous Arg-rich a6-helices and loop 1 motifs (FIG. 14C). Interestingly, AID was shown to bind G-quadraplex DNA structures using this basic region (Qiao et al., 2017). In addition, full length A3B had lower activity in an APOBEC-Mediated Base-Editing Reporter (“AMBER”) assay, whereas A3Bctd was much more active (see, U.S. application Ser. No. 16/035,286). Since the N-terminal domain is known to bind RNA (Xiao et al. 2017, supra), A3Bctd essentially is an RNA binding mutant.

Example 9—Hyperactive APOBEC Variants Show Increased Targeted DNA Editing Activity

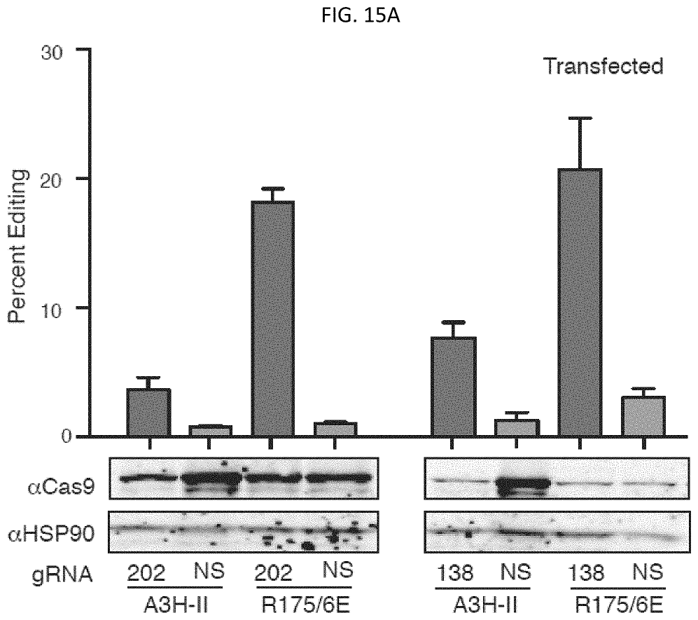

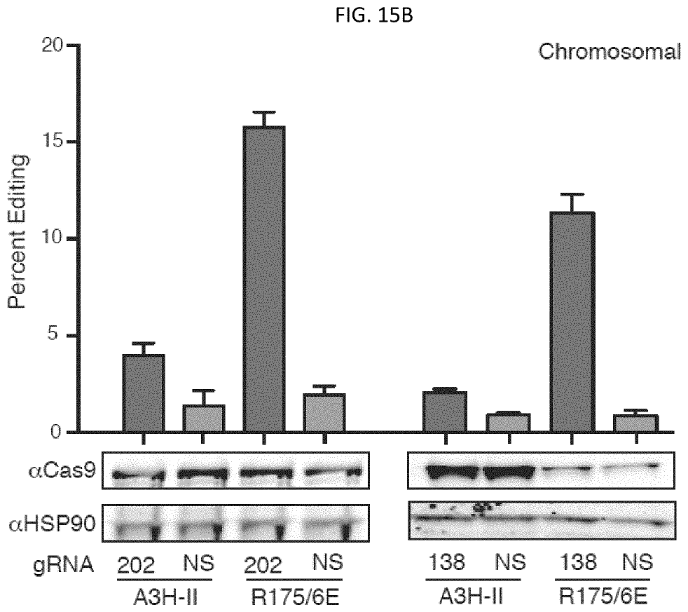

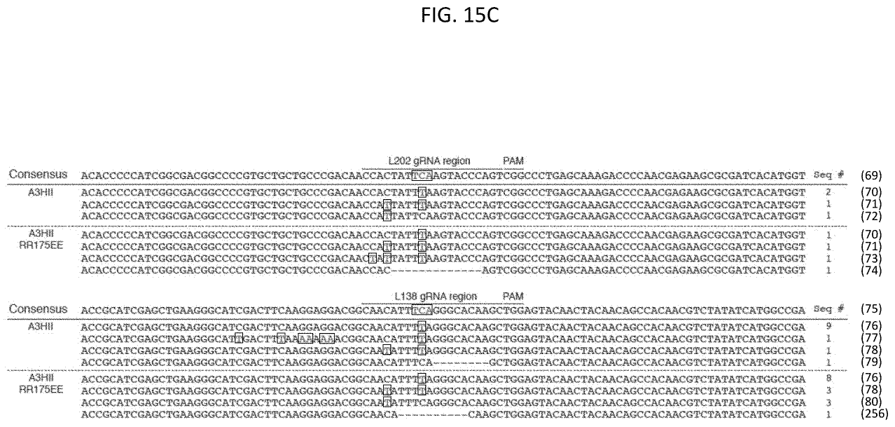

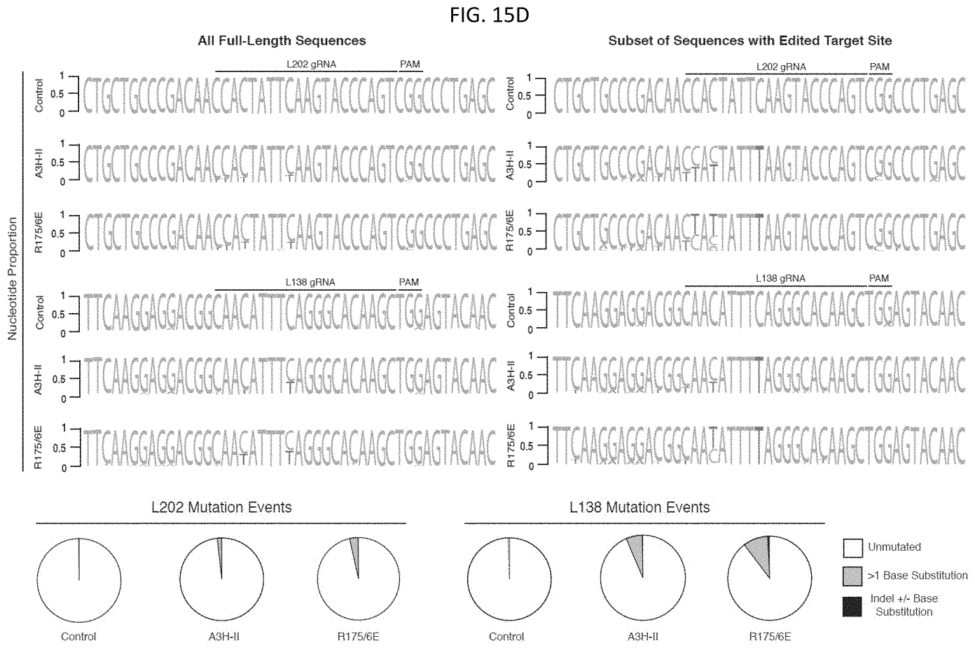

The targeted, single base editing activity of A3H-II editosomes and the otherwise identical R175/6E RNA binding mutant was assessed using an APOBEC-mediated base-editing reporter (AMBER) system. These studies showed that the R175/6E mutant was 3.1 to 5.5 fold more active than the editosome containing wild-type A3H (FIGS. 15A and 15B). Sanger and MiSeq DNA sequencing showed mostly on-target editing events and, interestingly, wild-type A3H-II editosomes also triggered lower rates of indel formation (FIGS. 15C and 5D). Thus RNA binding activity was clearly inhibitory for targeted DNA editing.

In summary, the studies described herein showed that A3H binds RNA through a RNA duplex-mediated dimerization mechanism. The RNA binding domain was identified through mutagenesis experiments, which yielded a group of A3H mutants with E. coli hypermutator activity, RNase A-independent DNA deaminase activity in 293T cell extracts, and monomeric size exclusion profiles. Two independent x-ray crystal structures of human A3H, a fully active K52E enzyme and an E56A derivative, demonstrate that all of the amino acid substitutions that confer these unique properties map to a positively charged protein surface that binds duplex RNA. The physiological importance of the RNA binding domain of A3H is evidenced by separation-of-function mutants showing deficiencies in cytoplasmic localization and HIV-1 encapsidation, while clearly retaining robust DNA deaminase activity. A related surprise is the observation that A3H RNA binding activity is more important than DNA deaminase activity for the overall HIV-1 restriction mechanism. These results therefore provide molecular and structural explanations for a previously unclear step of the HIV-1 restriction mechanism in which interactions with duplex RNA are essential for both cytoplasmic localization and packaging into HIV-1 particles.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A variant apolipoprotein B mRNA-editing complex (APOBEC) polypeptide comprising an amino acid sequence having one or more amino acid substitutions as compared to a reference APOBEC amino acid sequence, wherein said variant APOBEC polypeptide has reduced RNA binding, reduced oligomerization, or reduced RNA binding and reduced oligomerization, as compared to a polypeptide having the reference APOBEC amino acid sequence.

2. The variant APOBEC polypeptide of claim 1, wherein the polypeptide is an APOBEC3H (A3H) polypeptide, and wherein the reference APOBEC amino acid sequence is set forth in SEQ ID NO:81.

3. The variant APOBEC polypeptide of claim 2, wherein the one or more amino acid substitutions are at one or more of positions corresponding to positions 18, 20, 114, 115, 171, 172, 173, 175, 176, and 179 of SEQ ID NO:81.

4. The variant APOBEC polypeptide of claim 3, wherein the variant polypeptide further comprises a substitution at position 52 of SEQ ID NO:81.

5. The variant APOBEC polypeptide of claim 3, comprising a glutamic acid substituted at position 175, a glutamic acid substituted at position 176, or a glutamic acid substituted at position 175 and a glutamic acid substituted at position 176.

6. A fusion polypeptide comprising:
(a) a first portion that comprises a variant APOBEC polypeptide comprising an amino acid sequence having one or more amino acid substitutions as compared to a reference APOBEC amino acid sequence, wherein said variant APOBEC polypeptide has reduced RNA binding, reduced oligomerization, or reduced RNA binding and reduced oligomerization, as compared to a polypeptide having the reference APOBEC amino acid sequence; and
(b) a second portion that comprises a Cas9 polypeptide having the ability to complex with a guide RNA (gRNA), but lacking nuclease activity.

7. The fusion polypeptide of claim 6, wherein the variant APOBEC polypeptide is fused to the N-terminus of the Cas9 polypeptide or to the C-terminus of the Cas9 polypeptide.

8. The fusion polypeptide of claim 6, wherein the variant APOBEC polypeptide is coupled to the Cas9 polypeptide via a linker.

9. The fusion polypeptide of claim 6, wherein the fusion polypeptide comprises a third portion that comprises an additional functional domain from an inhibitor of uracil DNA glycosylase.

10. The fusion polypeptide of claim 6, wherein the variant APOBEC polypeptide is an A3H polypeptide, and wherein the reference APOBEC amino acid sequence is set forth in SEQ ID NO:81.

11. The fusion polypeptide of claim 10, wherein the one or more amino acid substitutions within the variant APOBEC polypeptide are at one or more of the positions corresponding to positions 18, 20, 114, 115, 171, 172, 173, 175, 176, and 179 of SEQ ID NO:81.

12. The fusion polypeptide of claim 11, wherein the variant APOBEC polypeptide further comprises a substitution at position 52 of SEQ ID NO:81.

13. The fusion polypeptide of claim 10, wherein the variant APOBEC polypeptide comprises a glutamic acid substituted at position 175, a glutamic acid substituted at position 176, or a glutamic acid substituted at position 175 and a glutamic acid substituted at position 176.

14. A nucleic acid molecule encoding the variant APOBEC polypeptide of claim 1 or the fusion polypeptide of claim 6.

15. The nucleic acid molecule of claim 14, further comprising (i) a sequence encoding a Clustered Regularly Interspersed Short Palindromic Repeats RNA (crRNA) and a sequence encoding a trans-activating crRNA (tracrRNA), or (ii) a sequence encoding a gRNA.

16. A method for targeted modification of a selected DNA sequence, comprising contacting the DNA sequence with:
(a) a fusion polypeptide that comprises
(i) a first portion comprising a variant APOBEC polypeptide comprising an amino acid sequence having one or more amino acid substitutions as compared to a reference APOBEC amino acid sequence, wherein said variant APOBEC polypeptide has reduced RNA binding, reduced oligomerization, or reduced RNA binding and reduced oligomerization, as compared to a polypeptide having the reference APOBEC amino acid sequence, and
(ii) a second portion comprising a Cas9 polypeptide having the ability to complex with a crRNA or a gRNA, but lacking nuclease activity; and
(b) a nucleic acid comprising a crRNA sequence and a tracrRNA sequence targeted to the selected sequence, or comprising a gRNA sequence,
such that the nucleic acid complexes with the fusion polypeptide and directs the fusion polypeptide to the selected sequence, wherein the method comprises contacting the DNA sequence with the fusion polypeptide and the nucleic acid in an amount effective for deamination of a deoxycytidine within the selected DNA sequence.

17. The method of claim 16, wherein the fusion polypeptide comprises a third portion that comprises an additional functional domain from an inhibitor of uracil DNA glycosylase.

18. The method of claim 16, wherein the variant APOBEC polypeptide is an A3H polypeptide, and wherein the reference APOBEC amino acid sequence is set forth in SEQ ID NO:81.

19. The method of claim 18, wherein the one or more amino acid substitutions within the variant APOBEC polypeptide are at one or more of the positions corresponding to positions 18, 20, 114, 115, 171, 172, 173, 175, 176, and 179 of SEQ ID NO:81.

20. The method of claim 19, wherein the variant APOBEC polypeptide further comprises a substitution at position 52 of SEQ ID NO:81.

21. The method of claim 18, wherein the variant APOBEC polypeptide comprises a glutamic acid substituted at position 175, a glutamic acid substituted at position 176, or a glutamic acid substituted at position 175 and a glutamic acid substituted at position 176.
